SEQUENCE LISTING (401 sequences)

SEQ ID NO: 1 - protein, 9 aa, synthetic construct
  REGION 1..9: note = synthetic peptide
IMVAFKGVW

SEQ ID NO: 2 - protein, 10 aa, synthetic construct
  REGION 1..10: note = synthetic peptide
NIMVAFKGVW

SEQ ID NO: 3 - protein, 11 aa, synthetic construct
  REGION 1..11: note = synthetic peptide
ENIMVAFKGVW

SEQ ID NO: 4 - protein, 12 aa, synthetic construct
  REGION 1..12: note = synthetic peptide
RENIMVAFKGVW

SEQ ID NO: 5 - protein, 10 aa, synthetic construct
  REGION 1..10: note = synthetic peptide
IMVAFKGVWT

SEQ ID NO: 6 - protein, 11 aa, synthetic construct
  REGION 1..11: note = synthetic peptide
NIMVAFKGVWT

SEQ ID NO: 7 - protein, 12 aa, synthetic construct
  REGION 1..12: note = synthetic peptide
ENIMVAFKGVWT

SEQ ID NO: 8 - protein, 13 aa, synthetic construct
  REGION 1..13: note = synthetic peptide
RENIMVAFKGVWT

SEQ ID NO: 9 - protein, 11 aa, synthetic construct
  REGION 1..11: note = synthetic peptide
IMVAFKGVWTQ

SEQ ID NO: 10 - protein, 12 aa, synthetic construct
  REGION 1..12: note = synthetic peptide
NIMVAFKGVWTQ

SEQ ID NO: 11 - protein, 13 aa, synthetic construct
  REGION 1..13: note = synthetic peptide
ENIMVAFKGVWTQ

SEQ ID NO: 12 - protein, 14 aa, synthetic construct
  REGION 1..14: note = synthetic peptide
RENIMVAFKGVWTQ

SEQ ID NO: 13 - protein, 12 aa, synthetic construct
  REGION 1..12: note = synthetic peptide
IMVAFKGVWTQA

SEQ ID NO: 14 - protein, 13 aa, synthetic construct
  REGION 1..13: note = synthetic peptide
NIMVAFKGVWTQA

SEQ ID NO: 15 - protein, 14 aa, synthetic construct
  REGION 1..14: note = synthetic peptide
ENIMVAFKGVWTQA

SEQ ID NO: 16 - protein, 15 aa, synthetic construct
  REGION 1..15: note = synthetic peptide
RENIMVAFKGVWTQA

SEQ ID NO: 17 - protein, 9 aa, synthetic construct
  REGION 1..9: note = synthetic peptide
LPVDMVLIS

SEQ ID NO: 18 - protein, 10 aa, synthetic construct
  REGION 1..10: note = synthetic peptide
PLPVDMVLIS

SEQ ID NO: 19 - protein, 11 aa, synthetic construct
  REGION 1..11: note = synthetic peptide
KPLPVDMVLIS

SEQ ID NO: 20 - protein, 12 aa, synthetic construct
  REGION 1..12: note = synthetic peptide
EKPLPVDMVLIS

SEQ ID NO: 21 - protein, 10 aa, synthetic construct
  REGION 1..10: note = synthetic peptide
LPVDMVLISL

SEQ ID NO: 22 - protein, 11 aa, synthetic construct
  REGION 1..11: note = synthetic peptide
PLPVDMVLISL

SEQ ID NO: 23 - protein, 12 aa, synthetic construct
  REGION 1..12: note = synthetic peptide
KPLPVDMVLISL

SEQ ID NO: 24 - protein, 13 aa, synthetic construct
  REGION 1..13: note = synthetic peptide
EKPLPVDMVLISL

SEQ ID NO: 25 - protein, 11 aa, synthetic construct
  REGION 1..11: note = synthetic peptide
  VARIANT 11: note = X = C or S
LPVDMVLISLX

SEQ ID NO: 26 - protein, 12 aa, synthetic construct
  REGION 1..12: note = synthetic peptide
  VARIANT 12: note = X = C or S
PLPVDMVLISLX

SEQ ID NO: 27 - protein, 13 aa, synthetic construct
  REGION 1..13: note = synthetic peptide
  VARIANT 13: note = X = C or S
KPLPVDMVLISLX

SEQ ID NO: 28 - protein, 14 aa, synthetic construct
  REGION 1..14: note = synthetic peptide
  VARIANT 14: note = X = C or S
EKPLPVDMVLISLX

SEQ ID NO: 29 - protein, 12 aa, synthetic construct
  REGION 1..12: note = synthetic peptide
  VARIANT 11: note = X = C or S
LPVDMVLISLXF

SEQ ID NO: 30 - protein, 13 aa, synthetic construct
  REGION 1..13: note = synthetic peptide
  VARIANT 12: note = X = C or S
PLPVDMVLISLXF

SEQ ID NO: 31 - protein, 14 aa, synthetic construct
  REGION 1..14: note = synthetic peptide
  VARIANT 13: note = X = C or S
KPLPVDMVLISLXF

SEQ ID NO: 32 - protein, 15 aa, synthetic construct
  REGION 1..15: note = synthetic peptide
  VARIANT 14: note = X = C or S
EKPLPVDMVLISLXF

SEQ ID NO: 33 - protein, 9 aa, synthetic construct
  REGION 1..9: note = synthetic peptide
  VARIANT 3: note = X = C or S
MVXTRKISI

SEQ ID NO: 34 - protein, 10 aa, synthetic construct
  REGION 1..10: note = synthetic peptide
  VARIANT 4: note = X = C or S
AMVXTRKISI

SEQ ID NO: 35 - protein, 11 aa, synthetic construct
  REGION 1..11: note = synthetic peptide
  VARIANT 5: note = X = C or S
VAMVXTRKISI

SEQ ID NO: 36 - protein, 12 aa, synthetic construct
  REGION 1..12: note = synthetic peptide
  VARIANT 6: note = X = C or S
TVAMVXTRKISI

SEQ ID NO: 37 - protein, 13 aa, synthetic construct
  REGION 1..13: note = synthetic peptide
  VARIANT 7: note = X = C or S
VTVAMVXTRKISI

SEQ ID NO: 38 - protein, 14 aa, synthetic construct
  REGION 1..14: note = synthetic peptide
  VARIANT 8: note = X = C or S
AVTVAMVXTRKISI

SEQ ID NO: 39 - protein, 11 aa, synthetic construct
  REGION 1..11: note = synthetic peptide
  VARIANT 4: note = X = C or S
AMVXTRKISIA

SEQ ID NO: 40 - protein, 12 aa, synthetic construct
  REGION 1..12: note = synthetic peptide
  VARIANT 5: note = X = C or S
VAMVXTRKISIA

SEQ ID NO: 41 - protein, 13 aa, synthetic construct
  REGION 1..13: note = synthetic peptide
  VARIANT 6: note = X = C or S
TVAMVXTRKISIA

SEQ ID NO: 42 - protein, 14 aa, synthetic construct
  REGION 1..14: note = synthetic peptide
  VARIANT 7: note = X = C or S
VTVAMVXTRKISIA

SEQ ID NO: 43 - protein, 15 aa, synthetic construct
  REGION 1..15: note = synthetic peptide
  VARIANT 8: note = X = C or S
AVTVAMVXTRKISIA

SEQ ID NO: 44 - protein, 9 aa, synthetic construct
  REGION 1..9: note = synthetic peptide
ISIAKSVFY

SEQ ID NO: 45 - protein, 10 aa, synthetic construct
  REGION 1..10: note = synthetic peptide
KISIAKSVFY

SEQ ID NO: 46 - protein, 11 aa, synthetic construct
  REGION 1..11: note = synthetic peptide
RKISIAKSVFY

SEQ ID NO: 47 - protein, 12 aa, synthetic construct
  REGION 1..12: note = synthetic peptide
TRKISIAKSVFY

SEQ ID NO: 48 - protein, 10 aa, synthetic construct
  REGION 1..10: note = synthetic peptide
ISIAKSVFYI

SEQ ID NO: 49 - protein, 11 aa, synthetic construct
  REGION 1..11: note = synthetic peptide
KISIAKSVFYI

SEQ ID NO: 50 - protein, 12 aa, synthetic construct
  REGION 1..12: note = synthetic peptide
RKISIAKSVFYI

SEQ ID NO: 51 - protein, 13 aa, synthetic construct
  REGION 1..13: note = synthetic peptide
TRKISIAKSVFYI

SEQ ID NO: 52 - protein, 11 aa, synthetic construct
  REGION 1..11: note = synthetic peptide
ISIAKSVFYIA

SEQ ID NO: 53 - protein, 12 aa, synthetic construct
  REGION 1..12: note = synthetic peptide
KISIAKSVFYIA

SEQ ID NO: 54 - protein, 13 aa, synthetic construct
  REGION 1..13: note = synthetic peptide
RKISIAKSVFYIA

SEQ ID NO: 55 - protein, 14 aa, synthetic construct
  REGION 1..14: note = synthetic peptide
TRKISIAKSVFYIA

SEQ ID NO: 56 - protein, 12 aa, synthetic construct
  REGION 1..12: note = synthetic peptide
ISIAKSVFYIAA

SEQ ID NO: 57 - protein, 13 aa, synthetic construct
  REGION 1..13: note = synthetic peptide
KISIAKSVFYIAA

SEQ ID NO: 58 - protein, 14 aa, synthetic construct
  REGION 1..14: note = synthetic peptide
RKISIAKSVFYIAA

SEQ ID NO: 59 - protein, 15 aa, synthetic construct
  REGION 1..15: note = synthetic peptide
TRKISIAKSVFYIAA

SEQ ID NO: 60 - protein, 9 aa, synthetic construct
  REGION 1..9: note = synthetic peptide
LIITFQLVF

SEQ ID NO: 61 - protein, 10 aa, synthetic construct
  REGION 1..10: note = synthetic peptide
ELIITFQLVF

SEQ ID NO: 62 - protein, 11 aa, synthetic construct
  REGION 1..11: note = synthetic peptide
VELIITFQLVF

SEQ ID NO: 63 - protein, 12 aa, synthetic construct
  REGION 1..12: note = synthetic peptide
LVELIITFQLVF

SEQ ID NO: 64 - protein, 10 aa, synthetic construct
  REGION 1..10: note = synthetic peptide
LIITFQLVFT

SEQ ID NO: 65 - protein, 11 aa, synthetic construct
  REGION 1..11: note = synthetic peptide
ELIITFQLVFT

SEQ ID NO: 66 - protein, 12 aa, synthetic construct
  REGION 1..12: note = synthetic peptide
VELIITFQLVFT

SEQ ID NO: 67 - protein, 13 aa, synthetic construct
  REGION 1..13: note = synthetic peptide
LVELIITFQLVFT

SEQ ID NO: 68 - protein, 11 aa, synthetic construct
  REGION 1..11: note = synthetic peptide
LIITFQLVFTI

SEQ ID NO: 69 - protein, 12 aa, synthetic construct
  REGION 1..12: note = synthetic peptide
ELIITFQLVFTI

SEQ ID NO: 70 - protein, 13 aa, synthetic construct
  REGION 1..13: note = synthetic peptide
VELIITFQLVFTI

SEQ ID NO: 71 - protein, 14 aa, synthetic construct
  REGION 1..14: note = synthetic peptide
LVELIITFQLVFTI

SEQ ID NO: 72 - protein, 9 aa, synthetic construct
  REGION 1..9: note = synthetic peptide
  VARIANT 5: note = X= C or S
IFASXDSKR

SEQ ID NO: 73 - protein, 10 aa, synthetic construct
  REGION 1..10: note = synthetic peptide
  VARIANT 6: note = X= C or S
TIFASXDSKR

SEQ ID NO: 74 - protein, 11 aa, synthetic construct
  REGION 1..11: note = synthetic peptide
  VARIANT 7: note = X= C or S
FTIFASXDSKR

SEQ ID NO: 75 - protein, 12 aa, synthetic construct
  REGION 1..12: note = synthetic peptide
  VARIANT 8: note = X=C or S
VFTIFASXDSKR

SEQ ID NO: 76 - protein, 10 aa, synthetic construct
  REGION 1..10: note = synthetic peptide
  VARIANT 5: note = X= C or S
IFASXDSKRT

SEQ ID NO: 77 - protein, 11 aa, synthetic construct
  REGION 1..11: note = synthetic peptide
  VARIANT 6: note = X= C or S
TIFASXDSKRT

SEQ ID NO: 78 - protein, 12 aa, synthetic construct
  REGION 1..12: note = synthetic peptide
  VARIANT 7: note = X= C or S
FTIFASXDSKRT

SEQ ID NO: 79 - protein, 13 aa, synthetic construct
  REGION 1..13: note = synthetic peptide
  VARIANT 8: note = X= C or S
VFTIFASXDSKRT

SEQ ID NO: 80 - protein, 11 aa, synthetic construct
  REGION 1..11: note = synthetic peptide
  VARIANT 5: note = X= C or S
IFASXDSKRTD

SEQ ID NO: 81 - protein, 12 aa, synthetic construct
  REGION 1..12: note = synthetic peptide
  VARIANT 6: note = X= C or S
TIFASXDSKRTD

SEQ ID NO: 82 - protein, 13 aa, synthetic construct
  REGION 1..13: note = synthetic peptide
  VARIANT 7: note = X= C or S
FTIFASXDSKRTD

SEQ ID NO: 83 - protein, 14 aa, synthetic construct
  REGION 1..14: note = synthetic peptide
  VARIANT 8: note = X= C or S
VFTIFASXDSKRTD

SEQ ID NO: 84 - protein, 12 aa, synthetic construct
  REGION 1..12: note = synthetic peptide
  VARIANT 5: note = X= C or S
IFASXDSKRTDV

SEQ ID NO: 85 - protein, 13 aa, synthetic construct
  REGION 1..13: note = synthetic peptide
  VARIANT 6: note = X= C or S
TIFASXDSKRTDV

SEQ ID NO: 86 - protein, 14 aa, synthetic construct
  REGION 1..14: note = synthetic peptide
  VARIANT 7: note = X= C or S
FTIFASXDSKRTDV

SEQ ID NO: 87 - protein, 15 aa, synthetic construct
  REGION 1..15: note = synthetic peptide
  VARIANT 8: note = X= C or S
VFTIFASXDSKRTDV

SEQ ID NO: 88 - protein, 9 aa, synthetic construct
  REGION 1..9: note = synthetic peptide
INYTGASMN

SEQ ID NO: 89 - protein, 10 aa, synthetic construct
  REGION 1..10: note = synthetic peptide
AINYTGASMN

SEQ ID NO: 90 - protein, 11 aa, synthetic construct
  REGION 1..11: note = synthetic peptide
FAINYTGASMN

SEQ ID NO: 91 - protein, 12 aa, synthetic construct
  REGION 1..12: note = synthetic peptide
LFAINYTGASMN

SEQ ID NO: 92 - protein, 10 aa, synthetic construct
  REGION 1..10: note = synthetic peptide
INYTGASMNP

SEQ ID NO: 93 - protein, 11 aa, synthetic construct
  REGION 1..11: note = synthetic peptide
AINYTGASMNP

SEQ ID NO: 94 - protein, 12 aa, synthetic construct
  REGION 1..12: note = synthetic peptide
FAINYTGASMNP

SEQ ID NO: 95 - protein, 13 aa, synthetic construct
  REGION 1..13: note = synthetic peptide
LFAINYTGASMNP

SEQ ID NO: 96 - protein, 11 aa, synthetic construct
  REGION 1..11: note = synthetic peptide
INYTGASMNPA

SEQ ID NO: 97 - protein, 12 aa, synthetic construct
  REGION 1..12: note = synthetic peptide
AINYTGASMNPA

SEQ ID NO: 98 - protein, 13 aa, synthetic construct
  REGION 1..13: note = synthetic peptide
FAINYTGASMNPA

SEQ ID NO: 99 - protein, 14 aa, synthetic construct
  REGION 1..14: note = synthetic peptide
LFAINYTGASMNPA

SEQ ID NO: 100 - protein, 12 aa, synthetic construct
  REGION 1..12: note = synthetic peptide
INYTGASMNPAR

SEQ ID NO: 101 - protein, 13 aa, synthetic construct
  REGION 1..13: note = synthetic peptide
AINYTGASMNPAR

SEQ ID NO: 102 - protein, 14 aa, synthetic construct
  REGION 1..14: note = synthetic peptide
FAINYTGASMNPAR

SEQ ID NO: 103 - protein, 15 aa, synthetic construct
  REGION 1..15: note = synthetic peptide
LFAINYTGASMNPAR

SEQ ID NO: 104 - protein, 9 aa, synthetic construct
  REGION 1..9: note = synthetic peptide
  VARIANT 2: note = X = C or S
FXPDVEFKR

SEQ ID NO: 105 - protein, 10 aa, synthetic construct
  REGION 1..10: note = synthetic peptide
  VARIANT 3: note = X = C or S
VFXPDVEFKR

SEQ ID NO: 106 - protein, 11 aa, synthetic construct
  REGION 1..11: note = synthetic peptide
  VARIANT 4: note = X = C or S
YVFXPDVEFKR

SEQ ID NO: 107 - protein, 12 aa, synthetic construct
  REGION 1..12: note = synthetic peptide
  VARIANT 5: note = X = C or S
EYVFXPDVEFKR

SEQ ID NO: 108 - protein, 10 aa, synthetic construct
  REGION 1..10: note = synthetic peptide
  VARIANT 2: note = X = C or S
FXPDVEFKRR

SEQ ID NO: 109 - protein, 11 aa, synthetic construct
  REGION 1..11: note = synthetic peptide
  VARIANT 3: note = X = C or S
VFXPDVEFKRR

SEQ ID NO: 110 - protein, 12 aa, synthetic construct
  REGION 1..12: note = synthetic peptide
  VARIANT 4: note = X = C or S
YVFXPDVEFKRR

SEQ ID NO: 111 - protein, 13 aa, synthetic construct
  REGION 1..13: note = synthetic peptide
  VARIANT 5: note = X = C or S
EYVFXPDVEFKRR

SEQ ID NO: 112 - protein, 11 aa, synthetic construct
  REGION 1..11: note = synthetic peptide
  VARIANT 2: note = X = C or S
FXPDVEFKRRF

SEQ ID NO: 113 - protein, 12 aa, synthetic construct
  REGION 1..12: note = synthetic peptide
  VARIANT 3: note = X = C or S
VFXPDVEFKRRF

SEQ ID NO: 114 - protein, 13 aa, synthetic construct
  REGION 1..13: note = synthetic peptide
  VARIANT 4: note = X = C or S
YVFXPDVEFKRRF

SEQ ID NO: 115 - protein, 14 aa, synthetic construct
  REGION 1..14: note = synthetic peptide
  VARIANT 5: note = X = C or S
EYVFXPDVEFKRRF

SEQ ID NO: 116 - protein, 12 aa, synthetic construct
  REGION 1..12: note = synthetic peptide
  VARIANT 2: note = X = C or S
FXPDVEFKRRFK

SEQ ID NO: 117 - protein, 13 aa, synthetic construct
  REGION 1..13: note = synthetic peptide
  VARIANT 3: note = X = C or S
VFXPDVEFKRRFK

SEQ ID NO: 118 - protein, 14 aa, synthetic construct
  REGION 1..14: note = synthetic peptide
  VARIANT 4: note = X = C or S
YVFXPDVEFKRRFK

SEQ ID NO: 119 - protein, 15 aa, synthetic construct
  REGION 1..15: note = synthetic peptide
  VARIANT 5: note = X = C or S
EYVFXPDVEFKRRFK

SEQ ID NO: 120 - protein, 9 aa, synthetic construct
  REGION 1..9: note = synthetic peptide
VETDDLILK

SEQ ID NO: 121 - protein, 10 aa, synthetic construct
  REGION 1..10: note = synthetic peptide
QVETDDLILK

SEQ ID NO: 122 - protein, 11 aa, synthetic construct
  REGION 1..11: note = synthetic peptide
SQVETDDLILK

SEQ ID NO: 123 - protein, 12 aa, synthetic construct
  REGION 1..12: note = synthetic peptide
RSQVETDDLILK

SEQ ID NO: 124 - protein, 11 aa, synthetic construct
  REGION 1..11: note = synthetic peptide
QVETDDLILKP

SEQ ID NO: 125 - protein, 11 aa, synthetic construct
  REGION 1..11: note = synthetic peptide
QVETDDLILKP

SEQ ID NO: 126 - protein, 12 aa, synthetic construct
  REGION 1..12: note = synthetic peptide
SQVETDDLILKP

SEQ ID NO: 127 - protein, 13 aa, synthetic construct
  REGION 1..13: note = synthetic peptide
RSQVETDDLILKP

SEQ ID NO: 128 - protein, 11 aa, synthetic construct
  REGION 1..11: note = synthetic peptide
VETDDLILKPG

SEQ ID NO: 129 - protein, 12 aa, synthetic construct
  REGION 1..12: note = synthetic peptide
QVETDDLILKPG

SEQ ID NO: 130 - protein, 13 aa, synthetic construct
  REGION 1..13: note = synthetic peptide
SQVETDDLILKPG

SEQ ID NO: 131 - protein, 14 aa, synthetic construct
  REGION 1..14: note = synthetic peptide
RSQVETDDLILKPG

SEQ ID NO: 132 - protein, 12 aa, synthetic construct
  REGION 1..12: note = synthetic peptide
VETDDLILKPGV

SEQ ID NO: 133 - protein, 13 aa, synthetic construct
  REGION 1..13: note = synthetic peptide
QVETDDLILKPGV

SEQ ID NO: 134 - protein, 14 aa, synthetic construct
  REGION 1..14: note = synthetic peptide
SQVETDDLILKPGV

SEQ ID NO: 135 - protein, 15 aa, synthetic construct
  REGION 1..15: note = synthetic peptide
RSQVETDDLILKPGV

SEQ ID NO: 136 - protein, 323 aa, synthetic construct
  REGION 1..323: note = synthetic peptide
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 137 - protein, 21 aa, synthetic construct
  REGION 1..21: note = synthetic peptide
HCPYCRENIMVAFKGVWTQAK

SEQ ID NO: 138 - protein, 21 aa, synthetic construct
  REGION 1..21: note = synthetic peptide
KHCPYCKPLPVDMVLISLSFK

SEQ ID NO: 139 - protein, 19 aa, synthetic construct
  REGION 1..19: note = synthetic peptide
HCPYCTVAMVSTRKISIAK

SEQ ID NO: 140 - protein, 24 aa, synthetic construct
  REGION 1..24: note = synthetic peptide
KHCPYCAVTVAMVSTRKISIAKKK

SEQ ID NO: 141 - protein, 24 aa, synthetic construct
  REGION 1..24: note = synthetic peptide
KHCPYCAVTVAMVCTRKISIAKKK

SEQ ID NO: 142 - protein, 22 aa, synthetic construct
  REGION 1..22: note = synthetic peptide
HCPYCAVTVAMVCTRKISIAKK

SEQ ID NO: 143 - protein, 20 aa, synthetic construct
  REGION 1..20: note = synthetic peptide
HCPYCTRKISIAKSVFYIAA

SEQ ID NO: 144 - protein, 22 aa, synthetic construct
  REGION 1..22: note = synthetic peptide
HCPYCTRKISIAKSVFYIAAKK

SEQ ID NO: 145 - protein, 22 aa, synthetic construct
  REGION 1..22: note = synthetic peptide
KHCPYCTRKSIAKSVFYIAAKK

SEQ ID NO: 146 - protein, 23 aa, synthetic construct
  REGION 1..23: note = synthetic peptide
HCPYCTRKISIAKSVFYIAAKKK

SEQ ID NO: 147 - protein, 21 aa, synthetic construct
  REGION 1..21: note = synthetic peptide
HCPYCLVELIITFQLVFTIDD

SEQ ID NO: 148 - protein, 21 aa, synthetic construct
  REGION 1..21: note = synthetic peptide
KHCPYCLVELIITFQLVFTIK

SEQ ID NO: 149 - protein, 21 aa, synthetic construct
  REGION 1..21: note = synthetic peptide
HCPYCVFTIFASSDSKRTDVK

SEQ ID NO: 150 - protein, 21 aa, synthetic construct
  REGION 1..21: note = synthetic peptide
HCPYCVFTIFASCDSKRTDVK

SEQ ID NO: 151 - protein, 22 aa, synthetic construct
  REGION 1..22: note = synthetic peptide
HCPYCVFTIFASCDSKRTDVKK

SEQ ID NO: 152 - protein, 22 aa, synthetic construct
  REGION 1..22: note = synthetic peptide
KHCPYCVFTIFASCDSKRTDVK

SEQ ID NO: 153 - protein, 21 aa, synthetic construct
  REGION 1..21: note = synthetic peptide
KHCPYCLFAINYTGASMNPAR

SEQ ID NO: 154 - protein, 20 aa, synthetic construct
  REGION 1..20: note = synthetic peptide
HCPYCEYVFSPDVEFKRRFK

SEQ ID NO: 155 - protein, 20 aa, synthetic construct
  REGION 1..20: note = synthetic peptide
HCPYCEYVFCPDVEFKRRFK

SEQ ID NO: 156 - protein, 21 aa, synthetic construct
  REGION 1..21: note = synthetic peptide
HCPYCRSQVETDDLILKPGVK

SEQ ID NO: 157 - protein, 4 aa, synthetic construct
  REGION 1..4: note = synthetic peptide
CPYC

SEQ ID NO: 158 - protein, 5 aa, synthetic construct
  REGION 1..5: note = synthetic peptide
HCPYC

SEQ ID NO: 159 - protein, 6 aa, synthetic construct
  REGION 1..6: note = synthetic peptide
KHCPYC

SEQ ID NO: 160 - protein, 5 aa, synthetic construct
  REGION 1..5: note = synthetic peptide
KCPYC

SEQ ID NO: 161 - protein, 5 aa, synthetic construct
  REGION 1..5: note = synthetic peptide
RCPYC

SEQ ID NO: 162 - protein, 6 aa, synthetic construct
  REGION 1..6: note = synthetic peptide
KKCPYC

SEQ ID NO: 163 - protein, 6 aa, synthetic construct
  REGION 1..6: note = synthetic peptide
KRCPYC

SEQ ID NO: 164 - protein, 4 aa, synthetic construct
  REGION 1..4: note = synthetic peptide
CHGC

SEQ ID NO: 165 - protein, 5 aa, synthetic construct
  REGION 1..5: note = synthetic peptide
HCGHC

SEQ ID NO: 166 - protein, 5 aa, synthetic construct
  REGION 1..5: note = synthetic peptide
KCGHC

SEQ ID NO: 167 - protein, 6 aa, synthetic construct
  REGION 1..6: note = synthetic peptide
KHCGHC

SEQ ID NO: 168 - protein, 5 aa, synthetic construct
  REGION 1..5: note = synthetic peptide
RCGHC

SEQ ID NO: 169 - protein, 6 aa, synthetic construct
  REGION 1..6: note = synthetic peptide
KKCGHC

SEQ ID NO: 170 - protein, 6 aa, synthetic construct
  REGION 1..6: note = synthetic peptide
KRCGHC

SEQ ID NO: 171 - protein, 12 aa, synthetic construct
  REGION 1..12: note = synthetic peptide
RENIMVAFKGVW

SEQ ID NO: 172 - protein, 11 aa, synthetic construct
  REGION 1..11: note = synthetic peptide
KPLPVDMVLIS

SEQ ID NO: 173 - protein, 12 aa, synthetic construct
  REGION 1..12: note = synthetic peptide
TVAMVSTRKISI

SEQ ID NO: 174 - protein, 14 aa, synthetic construct
  REGION 1..14: note = synthetic peptide
AVTVAMVSTRKISI

SEQ ID NO: 175 - protein, 14 aa, synthetic construct
  REGION 1..14: note = synthetic peptide
AVTVAMVCTRKISI

SEQ ID NO: 176 - protein, 12 aa, synthetic construct
  REGION 1..12: note = synthetic peptide
TRKISIAKSVFY

SEQ ID NO: 177 - protein, 12 aa, synthetic construct
  REGION 1..12: note = synthetic peptide
LVELIITFQLVF

SEQ ID NO: 178 - protein, 12 aa, synthetic construct
  REGION 1..12: note = synthetic peptide
VFTIFASSDSKR

SEQ ID NO: 179 - protein, 12 aa, synthetic construct
  REGION 1..12: note = synthetic peptide
VFTIFASCDSKR

SEQ ID NO: 180 - protein, 12 aa, synthetic construct
  REGION 1..12: note = synthetic peptide
LFAINYTGASMN

SEQ ID NO: 181 - protein, 12 aa, synthetic construct
  REGION 1..12: note = synthetic peptide
EYVFSPDVEFKR

SEQ ID NO: 182 - protein, 12 aa, synthetic construct
  REGION 1..12: note = synthetic peptide
EYVFCPDVEFKR

SEQ ID NO: 183 - protein, 12 aa, synthetic construct
  REGION 1..12: note = synthetic peptide
RSQVETDDLILK

SEQ ID NO: 184 - protein, 13 aa, synthetic construct
  REGION 1..13: note = synthetic peptide
IMVAFKGVWTQAK

SEQ ID NO: 185 - protein, 13 aa, synthetic construct
  REGION 1..13: note = synthetic peptide
LPVDMVLISLSFK

SEQ ID NO: 186 - protein, 11 aa, synthetic construct
  REGION 1..11: note = synthetic peptide
MVSTRKISIAK

SEQ ID NO: 187 - protein, 13 aa, synthetic construct
  REGION 1..13: note = synthetic peptide
MVSTRKISIAKKK

SEQ ID NO: 188 - protein, 13 aa, synthetic construct
  REGION 1..13: note = synthetic peptide
MVCTRKISIAKKK

SEQ ID NO: 189 - protein, 12 aa, synthetic construct
  REGION 1..12: note = synthetic peptide
MVCTRKISIAKK

SEQ ID NO: 190 - protein, 12 aa, synthetic construct
  REGION 1..12: note = synthetic peptide
ISIAKSVFYIAA

SEQ ID NO: 191 - protein, 14 aa, synthetic construct
  REGION 1..14: note = synthetic peptide
ISIAKSVFYIAAKK

SEQ ID NO: 192 - protein, 15 aa, synthetic construct
  REGION 1..15: note = synthetic peptide
ISIAKSVFYIAAKKK

SEQ ID NO: 193 - protein, 13 aa, synthetic construct
  REGION 1..13: note = synthetic peptide
LIITFQLVFTIDD

SEQ ID NO: 194 - protein, 12 aa, synthetic construct
  REGION 1..12: note = synthetic peptide
LIITFQLVFTIK

SEQ ID NO: 195 - protein, 13 aa, synthetic construct
  REGION 1..13: note = synthetic peptide
IFASSDSKRTDVK

SEQ ID NO: 196 - protein, 13 aa, synthetic construct
  REGION 1..13: note = synthetic peptide
IFASCDSKRTDVK

SEQ ID NO: 197 - protein, 14 aa, synthetic construct
  REGION 1..14: note = synthetic peptide
IFASCDSKRTDVKK

SEQ ID NO: 198 - protein, 13 aa, synthetic construct
  REGION 1..13: note = synthetic peptide
IFASCDSKRTDVK

SEQ ID NO: 199 - protein, 12 aa, synthetic construct
  REGION 1..12: note = synthetic peptide
INYTGASMNPAR

SEQ ID NO: 200 - protein, 12 aa, synthetic construct
  REGION 1..12: note = synthetic peptide
FSPDVEFKRRFK

SEQ ID NO: 201 - protein, 12 aa, synthetic construct
  REGION 1..12: note = synthetic peptide
FCPDVEFKRRFK

SEQ ID NO: 202 - protein, 13 aa, synthetic construct
  REGION 1..13: note = synthetic peptide
VETDDLILKPGVK

SEQ ID NO: 203 - protein, 16 aa, synthetic construct
  REGION 1..16: note = synthetic peptide
RENIMVAFKGVWTQAK

SEQ ID NO: 204 - protein, 15 aa, synthetic construct
  REGION 1..15: note = synthetic peptide
KPLPVDMVLISLSFK

SEQ ID NO: 205 - protein, 14 aa, synthetic construct
  REGION 1..14: note = synthetic peptide
TVAMVSTRKISIAK

SEQ ID NO: 206 - protein, 18 aa, synthetic construct
  REGION 1..18: note = synthetic peptide
AVTVAMVSTRKISIAKKK

SEQ ID NO: 207 - protein, 18 aa, synthetic construct
  REGION 1..18: note = synthetic peptide
AVTVAMVCTRKISIAKKK

SEQ ID NO: 208 - protein, 17 aa, synthetic construct
  REGION 1..17: note = synthetic peptide
AVTVAMVCTRKISIAKK

SEQ ID NO: 209 - protein, 15 aa, synthetic construct
  REGION 1..15: note = synthetic peptide
TRKISIAKSVFYIAA

SEQ ID NO: 210 - protein, 17 aa, synthetic construct
  REGION 1..17: note = synthetic peptide
TRKISIAKSVFYIAAKK

SEQ ID NO: 211 - protein, 18 aa, synthetic construct
  REGION 1..18: note = synthetic peptide
TRKISIAKSVFYIAAKKK

SEQ ID NO: 212 - protein, 16 aa, synthetic construct
  REGION 1..16: note = synthetic peptide
LVELIITFQLVFTIDD

SEQ ID NO: 213 - protein, 15 aa, synthetic construct
  REGION 1..15: note = synthetic peptide
LVELIITFQLVFTIK

SEQ ID NO: 214 - protein, 16 aa, synthetic construct
  REGION 1..16: note = synthetic peptide
VFTIFASSDSKRTDVK

SEQ ID NO: 215 - protein, 16 aa, synthetic construct
  REGION 1..16: note = synthetic peptide
VFTIFASCDSKRTDVK

SEQ ID NO: 216 - protein, 17 aa, synthetic construct
  REGION 1..17: note = synthetic peptide
VFTIFASCDSKRTDVKK

SEQ ID NO: 217 - protein, 16 aa, synthetic construct
  REGION 1..16: note = synthetic peptide
VFTIFASCDSKRTDVK

SEQ ID NO: 218 - protein, 15 aa, synthetic construct
  REGION 1..15: note = synthetic peptide
LFAINYTGASMNPAR

SEQ ID NO: 219 - protein, 15 aa, synthetic construct
  REGION 1..15: note = synthetic peptide
EYVFSPDVEFKRRFK

SEQ ID NO: 220 - protein, 15 aa, synthetic construct
  REGION 1..15: note = synthetic peptide
EYVFCPDVEFKRRFK

SEQ ID NO: 221 - protein, 16 aa, synthetic construct
  REGION 1..16: note = synthetic peptide
RSQVETDDLILKPGVK

SEQ ID NO: 222-226 - skipped (5 entries)

SEQ ID NO: 227 - protein, 14 aa, synthetic construct
  REGION 1..14: note = synthetic peptide
  VARIANT 2..3: note = Xaa can be any naturally occurring amino acid
  VARIANT 5: note = Xaa can be any naturally occurring amino acid
  VARIANT 7..8: note = Xaa can be any naturally occurring amino acid
  VARIANT 10: note = Xaa can be any naturally occurring amino acid
  VARIANT 12..13: note = Xaa can be any naturally occurring amino acid
CXXCXCXXCXCXXC

SEQ ID NO: 228 - protein, 12 aa, synthetic construct
  REGION 1..12: note = synthetic peptide
  VARIANT 2..3: note = Xaa can be any naturally occurring amino acid
  VARIANT 6..7: note = Xaa can be any naturally occurring amino acid
  VARIANT 10..11: note = Xaa can be any naturally occurring amino acid
CXXCCXXCCXXC

SEQ ID NO: 229 - protein, 10 aa, synthetic construct
  REGION 1..10: note = synthetic peptide
  VARIANT 2..3: note = Xaa can be any naturally occurring amino acid
  VARIANT 5..6: note = Xaa can be any naturally occurring amino acid
  VARIANT 8..9: note = Xaa can be any naturally occurring amino acid
CXXCXXCXXC

SEQ ID NO: 230 - protein, 10 aa, synthetic construct
  REGION 1..10: note = synthetic peptide
  VARIANT 2: note = Xaa can be any naturally occurring amino acid
  VARIANT 5: note = Xaa can be any naturally occurring amino acid
  VARIANT 8: note = Xaa can be any naturally occurring amino acid
CXCCXCCXCC

SEQ ID NO: 231 - protein, 4 aa, synthetic construct
  REGION 1..4: note = synthetic peptide
KKCC

SEQ ID NO: 232 - protein, 4 aa, synthetic construct
  REGION 1..4: note = synthetic peptide
RRCC

SEQ ID NO: 233 - protein, 4 aa, synthetic construct
  REGION 1..4: note = synthetic peptide
RKCC

SEQ ID NO: 234 - protein, 4 aa, synthetic construct
  REGION 1..4: note = synthetic peptide
KRCC

SEQ ID NO: 235 - skipped

SEQ ID NO: 236 - protein, 5 aa, synthetic construct
  REGION 1..5: note = synthetic peptide
  VARIANT 4: note = Xaa can be any naturally occurring amino acid
KKCXC

SEQ ID NO: 237 - skipped

SEQ ID NO: 238 - protein, 5 aa, synthetic construct
  REGION 1..5: note = synthetic peptide
  VARIANT 4: note = Xaa can be any naturally occurring amino acid
RRCXC

SEQ ID NO: 239 - protein, 5 aa, synthetic construct
  REGION 1..5: note = synthetic peptide
  VARIANT 4: note = Xaa can be any naturally occurring amino acid
RKCXC

SEQ ID NO: 240 - protein, 5 aa, synthetic construct
  REGION 1..5: note = synthetic peptide
  VARIANT 4: note = Xaa can be any naturally occurring amino acid
KRCXC

SEQ ID NO: 241 - protein, 4 aa, synthetic construct
  REGION 1..4: note = synthetic peptide
KCKC

SEQ ID NO: 242 - protein, 5 aa, synthetic construct
  REGION 1..5: note = synthetic peptide
KKCKC

SEQ ID NO: 243 - protein, 4 aa, synthetic construct
  REGION 1..4: note = synthetic peptide
KCRC

SEQ ID NO: 244 - protein, 5 aa, synthetic construct
  REGION 1..5: note = synthetic peptide
KKCRC

SEQ ID NO: 245 - protein, 4 aa, synthetic construct
  REGION 1..4: note = synthetic peptide
RCRC

SEQ ID NO: 246 - protein, 5 aa, synthetic construct
  REGION 1..5: note = synthetic peptide
RRCRC

SEQ ID NO: 247 - protein, 5 aa, synthetic construct
  REGION 1..5: note = synthetic peptide
RKCKC

SEQ ID NO: 248 - protein, 5 aa, synthetic construct
  REGION 1..5: note = synthetic peptide
KRCKC

SEQ ID NO: 249 - protein, 4 aa, synthetic construct
  REGION 1..4: note = synthetic peptide
RCKC

SEQ ID NO: 250 - protein, 5 aa, synthetic construct
  REGION 1..5: note = synthetic peptide
CRPYC

SEQ ID NO: 251 - protein, 6 aa, synthetic construct
  REGION 1..6: note = synthetic peptide
KCRPYC

SEQ ID NO: 252 - protein, 7 aa, synthetic construct
  REGION 1..7: note = synthetic peptide
KHCRPYC

SEQ ID NO: 253 - protein, 6 aa, synthetic construct
  REGION 1..6: note = synthetic peptide
RCRPYC

SEQ ID NO: 254 - protein, 6 aa, synthetic construct
  REGION 1..6: note = synthetic peptide
HCRPYC

SEQ ID NO: 255 - protein, 5 aa, synthetic construct
  REGION 1..5: note = synthetic peptide
CPRYC

SEQ ID NO: 256 - protein, 6 aa, synthetic construct
  REGION 1..6: note = synthetic peptide
KCPRYC

SEQ ID NO: 257 - protein, 6 aa, synthetic construct
  REGION 1..6: note = synthetic peptide
RCPRYC

SEQ ID NO: 258 - protein, 6 aa, synthetic construct
  REGION 1..6: note = synthetic peptide
HCPRYC

SEQ ID NO: 259 - protein, 5 aa, synthetic construct
  REGION 1..5: note = synthetic peptide
CPYRC

SEQ ID NO: 260 - protein, 6 aa, synthetic construct
  REGION 1..6: note = synthetic peptide
KCPYRC

SEQ ID NO: 261 - protein, 6 aa, synthetic construct
  REGION 1..6: note = synthetic peptide
RCPYRC

SEQ ID NO: 262 - protein, 6 aa, synthetic construct
  REGION 1..6: note = synthetic peptide
HCPYRC

SEQ ID NO: 263 - protein, 5 aa, synthetic construct
  REGION 1..5: note = synthetic peptide
CKPYC

SEQ ID NO: 264 - protein, 6 aa, synthetic construct
  REGION 1..6: note = synthetic peptide
KCKPYC

SEQ ID NO: 265 - protein, 6 aa, synthetic construct
  REGION 1..6: note = synthetic peptide
RCKPYC

SEQ ID NO: 266 - protein, 6 aa, synthetic construct
  REGION 1..6: note = synthetic peptide
HCKPYC

SEQ ID NO: 267 - protein, 5 aa, synthetic construct
  REGION 1..5: note = synthetic peptide
CPKYC

SEQ ID NO: 268 - protein, 6 aa, synthetic construct
  REGION 1..6: note = synthetic peptide
KCPKYC

SEQ ID NO: 269 - protein, 6 aa, synthetic construct
  REGION 1..6: note = synthetic peptide
RCPKYC

SEQ ID NO: 270 - protein, 6 aa, synthetic construct
  REGION 1..6: note = synthetic peptide
HCPKYC

SEQ ID NO: 271 - protein, 5 aa, synthetic construct
  REGION 1..5: note = synthetic peptide
CPYKC

SEQ ID NO: 272 - protein, 6 aa, synthetic construct
  REGION 1..6: note = synthetic peptide
KCPYKC

SEQ ID NO: 273 - protein, 6 aa, synthetic construct
  REGION 1..6: note = synthetic peptide
RCPYKC

SEQ ID NO: 274 - protein, 6 aa, synthetic construct
  REGION 1..6: note = synthetic peptide
HCPYKC

SEQ ID NO: 275 - skipped

SEQ ID NO: 276 - protein, 4 aa, synthetic construct
  REGION 1..4: note = synthetic peptide
LAVL

SEQ ID NO: 277 - protein, 4 aa, synthetic construct
  REGION 1..4: note = synthetic peptide
TVQA

SEQ ID NO: 278 - protein, 4 aa, synthetic construct
  REGION 1..4: note = synthetic peptide
GAVH

SEQ ID NO: 279-291 - skipped (13 entries)

SEQ ID NO: 292 - protein, 5 aa, synthetic construct
  REGION 1..5: note = synthetic peptide
PAFPL

SEQ ID NO: 293 - protein, 5 aa, synthetic construct
  REGION 1..5: note = synthetic peptide
DQGGE

SEQ ID NO: 294 - protein, 5 aa, synthetic construct
  REGION 1..5: note = synthetic peptide
  VARIANT 1: note = Xaa can be any naturally occurring amino acid
XAFPL

SEQ ID NO: 295 - protein, 5 aa, synthetic construct
  REGION 1..5: note = synthetic peptide
  VARIANT 2: note = Xaa can be any naturally occurring amino acid
PXFPL

SEQ ID NO: 296 - protein, 5 aa, synthetic construct
  REGION 1..5: note = synthetic peptide
  VARIANT 3: note = Xaa can be any naturally occurring amino acid
PAXPL

SEQ ID NO: 297 - protein, 5 aa, synthetic construct
  REGION 1..5: note = synthetic peptide
  VARIANT 4: note = Xaa can be any naturally occurring amino acid
PAFXL

SEQ ID NO: 298 - protein, 5 aa, synthetic construct
  REGION 1..5: note = synthetic peptide
  VARIANT 5: note = Xaa can be any naturally occurring amino acid
PAFPX

SEQ ID NO: 299 - protein, 5 aa, synthetic construct
  REGION 1..5: note = synthetic peptide
  VARIANT 1: note = Xaa can be any naturally occurring amino acid
XQGGE

SEQ ID NO: 300 - protein, 5 aa, synthetic construct
  REGION 1..5: note = synthetic peptide
  VARIANT 2: note = Xaa can be any naturally occurring amino acid
DXGGE

SEQ ID NO: 301 - protein, 5 aa, synthetic construct
  REGION 1..5: note = synthetic peptide
  VARIANT 3: note = Xaa can be any naturally occurring amino acid
DQXGE

SEQ ID NO: 302 - protein, 5 aa, synthetic construct
  REGION 1..5: note = synthetic peptide
  VARIANT 4: note = Xaa can be any naturally occurring amino acid
DQGXE

SEQ ID NO: 303 - protein, 5 aa, synthetic construct
  REGION 1..5: note = synthetic peptide
  VARIANT 5: note = Xaa can be any naturally occurring amino acid
DQGGX

SEQ ID NO: 304 - skipped

SEQ ID NO: 305 - protein, 6 aa, synthetic construct
  REGION 1..6: note = synthetic peptide
DIADKY

SEQ ID NO: 306 - protein, 6 aa, synthetic construct
  REGION 1..6: note = synthetic peptide
  VARIANT 1: note = Xaa can be any naturally occurring amino acid
XIADKY

SEQ ID NO: 307 - protein, 6 aa, synthetic construct
  REGION 1..6: note = synthetic peptide
  VARIANT 2: note = Xaa can be any naturally occurring amino acid
DXADKY

SEQ ID NO: 308 - protein, 6 aa, synthetic construct
  REGION 1..6: note = synthetic peptide
  VARIANT 3: note = Xaa can be any naturally occurring amino acid
DIXDKY

SEQ ID NO: 309 - protein, 6 aa, synthetic construct
  REGION 1..6: note = synthetic peptide
  VARIANT 4: note = Xaa can be any naturally occurring amino acid
DIAXKY

SEQ ID NO: 310 - protein, 6 aa, synthetic construct
  REGION 1..6: note = synthetic peptide
  VARIANT 5: note = Xaa can be any naturally occurring amino acid
DIADXY

SEQ ID NO: 311 - protein, 6 aa, synthetic construct
  REGION 1..6: note = synthetic peptide
  VARIANT 6: note = Xaa can be any naturally occurring amino acid
DIADKX

SEQ ID NO: 312-319 - skipped (8 entries)

SEQ ID NO: 320 - protein, 4 aa, synthetic construct
  REGION 1..4: note = synthetic peptide
HCRC

SEQ ID NO: 321 - protein, 5 aa, synthetic construct
  REGION 1..5: note = synthetic peptide
KHCRC

SEQ ID NO: 322-396 - skipped (75 entries)

SEQ ID NO: 397 - protein, 20 aa, synthetic construct
  REGION 1..20: note = synthetic peptide
AGGLYEYVFSPDVEFKRRFK

SEQ ID NO: 398 - protein, 20 aa, synthetic construct
  REGION 1..20: note = synthetic peptide
AGGLYEYVFCPDVEFKRRFK

SEQ ID NO: 399 - protein, 23 aa, synthetic construct
  REGION 1..23: note = synthetic peptide
KVAMVCTRKISIAKSVFYIAAKK

SEQ ID NO: 400 - protein, 18 aa, synthetic construct
  REGION 1..18: note = synthetic peptide
SIMVAFKGVWTQAFWKAV

SEQ ID NO: 401 - protein, 23 aa, synthetic construct
  REGION 1..23: note = synthetic peptide
HCPYCSIMVAFKGVWTQAFWKAV

(19) United States
(12) Patent Application Publication
(10) Pub. No.: US 2024/0228558 A9
(43) Pub. Date: Jul. 11, 2024

(54) PEPTIDES AND METHODS FOR THE TREATMENT OF NEUROMYELITIS OPTICA

(71) Applicant: IMCYSE SA, Angleur (Liège) (BE)

(72) Inventor: Milos ERAK, Bruxelles (BE)

(21) Appl. No.: 18/542,031

(22) Filed: Dec. 15, 2023

Related U.S. Application Data
(63) Continuation of application No. PCT/EP2022/067825, filed on Jun. 29, 2022.

(30) Foreign Application Priority Data
Jun. 29, 2021 (EP) 21182499.0

(51) Int. Cl. C07K 14/47; A61K 35/17; A61K 38/00; A61K 39/00; C07K 16/28

(52) U.S. Cl. CPC C07K 14/47; A61K 35/17; C07K 16/2803; C07K 16/2887; A61K 38/00; A61K 2039/505

(57) ABSTRACT

The invention relates to immunogenic peptides derived from Aquaporin 4 (AQP4) for use in the treatment of Neuromyelitis Optica Spectrum Disorders (NMOSD) and to the generation of cytolytic CD4+ T cells or NKT cells against antigen presenting cells that present the wild-type AQP4 epitope sequence.

### CROSS-REFERENCE TO RELATED APPLICATIONS

This application is the U.S. continuation of International Application No. PCT/EP2022/067825 filed Jun. 29, 2022, which designated the U.S. and claims priority to EP 21182499.0 filed Jun. 29, 2021, the entire contents of each of which are hereby incorporated by reference.

### REFERENCE TO SEQUENCE LISTING SUBMITTED ELECTRONICALLY

The content of the electronically submitted sequence listing (Name: 2752-155_Sequence_Listing.xml, Size: 413 KB, and Date of Creation: Dec. 15, 2023) is herein incorporated by reference in its entirety.

### FIELD OF THE INVENTION

The present invention relates to immunogenic peptides. In particular, the invention relates to immunogenic peptides comprising an oxidoreductase motif linked to a T cell epitope derived from Aquaporin-4 (AQP4) and cytolytic CD4+ T cells generated by these peptides for use in the treatment of anti-AQP4 diseases or Neuromyelitis Optica Spectrum Disorders (NMOSD), more particularly Neuromyelitis Optica (NMO).

### BACKGROUND

Several strategies have been described to prevent the generation of an unwanted immune response against an antigen. WO2008/017517 describes a new strategy using peptides comprising an MHC class II T cell epitope of a given antigenic protein and an oxidoreductase motif. These peptides convert CD4+ T cells into a cell type with cytolytic properties called cytolytic CD4+ T cells. These cells are capable to kill via triggering apoptosis those antigen presenting cells (APC), which present the antigen from which the peptide is derived. WO2008/017517 demonstrates this concept for allergies and auto-immune diseases such as type I diabetes.

WO2009101207 and Carlier et al. (2012) Plos one 7,10 e45366 further describe the antigen specific cytolytic cells in more detail. WO2016059236 discloses further modified peptides wherein an additional Histidine is present in the proximity of the oxidoreductase motif. WO2012069568 further discloses peptides comprising an NKT cell epitope of an antigenic protein and an oxidoreductase motif. These peptides are capable of eliciting activation of NKT cells, which represent a valuable approach for the treatment of many diseases such as infectious and autoimmune diseases or cancer. WO2017182528 describes the use of an immunogenic peptide comprising a MOG epitope for use in treating Multiple Sclerosis.

Neuromyelitis Optica (NMO) is an inflammatory disease of the central nervous system (CNS) that is characterized by severe attacks of optic neuritis (ON) and longitudinally extensive (transverse) myelitis (LE(T)M), which in some cases has a substantial overlap in clinical symptoms with (certain) MS subtypes, also called Neuromyelitis Optica Spectrum Disorder (NMOSD) (cf. e.g. Wingerchuk et al., 2007-Lancet Neurol 6:805-815; Kim et la., 2012-Neurology 78:1179-1185). Recent developments have led to the discovery of a disease-specific autoantibody, NMO-immunoglobulin G (NMO-IgG), and subsequent identification of the main target autoantigen, Aquaporin-4 (AQP4). This has helped to distinguished NMO as a distinct disease from multiple sclerosis (MS) in recent years. The NMOSD encompasses in principle all anti-AQP4 antibody seropositive patients with limited or inaugural forms of NMO and with specific brain abnormalities. It hence also includes anti-AQP4 antibody seropositive patients with other autoimmune disorders such as systemic lupus erythematosus and Sjögren syndrome.

Hence, novel and/or improved treatment strategies for AQP4 autoantigen-induced or anti-AQP4 antibody induced diseases such as NMOSD and NMO are needed.

### SUMMARY

The present invention provides novel peptides comprising epitopes derived from the Aquaporin-4 (AQP4) antigen for the treatment of demyelinating disorders such as but not limited to Neuromyelitis Optica (NMO), Neuromyelitis Optica Spectrum Disorders (NMOSD) or anti-AQP4 diseases in general. The peptides of the present invention have the advantage that they bind to HLA-DRB1*03:01 and/or HLA-DPB1*05:01; most preferably to HLA-DRB1*03:01. Stimulation of NMO patients cells with the peptides of the invention can induce specific CD4+ T cells with lytic properties towards APCs presenting AQP4 epitopes, thereby allowing to stop the autoimmune response targeting AQP4.

The invention therefore provides the following aspects:

Aspect 1. An isolated immunogenic peptide comprising:
an oxidoreductase motif with the sequence Zₘ-[CST]-Xₙ-C- or Zₘ-C-Xₙ-[CST]-, wherein n is an integer chosen from 0 to 6, preferably 0, 1, 2, or 3, wherein m is an integer selected from 0 to 3, wherein X is any amino acid, wherein Z is any amino acid, in which C stands for cysteine, S for serine, T for threonine; and
a T-cell epitope of Aquaporin-4 (AQP4), preferably human Aquaporin-4, more preferably Aquaporin-4 defined by the amino acid sequence as defined in SEQ ID NO: 136; wherein said oxidoreductase motif and said epitope are separated by a linker sequence of between 0 to 7 amino acids and optionally comprises a C-terminal flanking sequence of between 0 to 7 amino acids.

In one embodiment, said epitope is not a mouse Aquaporin-4 epitope, more specifically, said epitope is not mouse AQP4 epitope SIMVAFKGVWTQAFWKAV (SEQ ID NO: 400) and said immunogenic peptide is not

(SEQ ID NO: 401)
HCPYCSIMVAFKGVWTQAFWKAV.

In said general formula of the oxidoreductase motif, the hyphen (-) indicates the point of attachment of the oxidoreductase motif to the N-terminal end of the linker or the epitope, or to the C-terminal end of the linker or the T cell epitope.

In preferred embodiments, said oxidoreductase motif sequence is not occurring in the natural (wild-type) amino acid sequence of AQP4. In general this implies that the epitope is chosen such that within 11 amino acids N- or C-terminally adjacent to said epitope in the natural (wild-type) sequence of AQP4, no oxidoreductase motif as described herein occurs.

T cell epitopes of AQP4 can comprise or consist of any one or more of the following sequences (based on the AQP4 amino acid positions in SEQ ID NO: 136):

AQP4 region 19-33:

(SEQ ID NO: 1)
IMVAFKGVW
(SEQ ID NO: 2)
NIMVAFKGVW
(SEQ ID NO: 3)
ENIMVAFKGVW
(SEQ ID NO: 4)
RENIMVAFKGVW
(SEQ ID NO: 5)
IMVAFKGVWT
(SEQ ID NO: 6)
NIMVAFKGVWT
(SEQ ID NO: 7)
ENIMVAFKGVWT
(SEQ ID NO: 8)
RENIMVAFKGVWT
(SEQ ID NO: 9)
IMVAFKGVWTQ
(SEQ ID NO: 10)
NIMVAFKGVWTQ
(SEQ ID NO: 11)
ENIMVAFKGVWTQ
(SEQ ID NO: 12)
RENIMVAFKGVWTQ
(SEQ ID NO: 13)
IMVAFKGVWTQA
(SEQ ID NO: 14)
NIMVAFKGVWTQA
(SEQ ID NO: 15)
ENIMVAFKGVWTQA
(SEQ ID NO: 16)
RENIMVAFKGVWTQA

AQP4 region 64-77:

(SEQ ID NO: 17)
LPVDMVLIS
(SEQ ID NO: 18)
PLPVDMVLIS
(SEQ ID NO: 19)
KPLPVDMVLIS
(SEQ ID NO: 20)
EKPLPVDMVLIS
(SEQ ID NO: 21)
LPVDMVLISL
(SEQ ID NO: 22)
PLPVDMVLISL
(SEQ ID NO: 23)
KPLPVDMVLISL
(SEQ ID NO: 24)
EKPLPVDMVLISL
(SEQ ID NO: 25)
LPVDMVLISL[CS]
(SEQ ID NO: 26)
PLPVDMVLISL[CS]
(SEQ ID NO: 27)
KPLPVDMVLISL[CS]
(SEQ ID NO: 28)
EKPLPVDMVLISL[CS]
(SEQ ID NO: 29)
LPVDMVLISL[CS]F
(SEQ ID NO: 30)
PLPVDMVLISL[CS]F
(SEQ ID NO: 31)
VDMVLISL[CS]F
(SEQ ID NO: 32)
EKPLPVDMVLISL[CS]F

AQP4 region 101-114:

(SEQ ID NO: 33)
MV[CS]TRKISI
(SEQ ID NO: 34)
AMV[CS]TRKISI
(SEQ ID NO: 35)
VAMV[CS]TRKISI
(SEQ ID NO: 36)
TVAMV[CS]TRKISI
(SEQ ID NO: 37)
VTVAMV[CS]TRKISI
(SEQ ID NO: 38)
AVTVAMV[CS]TRKISI
(SEQ ID NO: 39)
AMV[CS]TRKISIA
(SEQ ID NO: 40)
VAMV[CS]TRKISIA
(SEQ ID NO: 41)
TVAMV[CS]TRKISIA
(SEQ ID NO: 42)
VTVAMV[CS]TRKISIA
(SEQ ID NO: 43)
AVTVAMV[CS]TRKISIA

AQP4 region 107-121:

(SEQ ID NO: 44)
ISIAKSVFY
(SEQ ID NO: 45)
KISIAKSVFY
(SEQ ID NO: 46)
RKISIAKSVFY
(SEQ ID NO: 47)
TRKISIAKSVFY
(SEQ ID NO: 48)
ISIAKSVFYI
(SEQ ID NO: 49)
KISIAKSVFYI
(SEQ ID NO: 50)
RKISIAKSVFYI
(SEQ ID NO: 51)
TRKISIAKSVFYI
(SEQ ID NO: 52)
ISIAKSVFYIA
(SEQ ID NO: 53)
KISIAKSVFYIA
(SEQ ID NO: 54)
RKISIAKSVFYIA
(SEQ ID NO: 55)
TRKISIAKSVFYIA
(SEQ ID NO: 56)
ISIAKSVFYIAA
(SEQ ID NO: 57)
KISIAKSVFYIAA
(SEQ ID NO: 58)
RKISIAKSVFYIAA
(SEQ ID NO: 59)
TRKISIAKSVFYIAA

AQP4 region 161-174:

(SEQ ID NO: 60
LIITFQLVF
(SEQ ID NO: 61)
ELIITFQLVF
(SEQ ID NO: 62)
VELIITFQLVF
(SEQ ID NO: 63)
LVELIITFQLVF
(SEQ ID NO: 64)
LIITFQLVFT
(SEQ ID NO: 65)
ELIITFQLVFT
(SEQ ID NO: 66)
VELIITFQLVFT
(SEQ ID NO: 67)
LVELIITFQLVFT
(SEQ ID NO: 68)
LIITFQLVFTI
(SEQ ID NO: 69)
ELIITFQLVFTI
(SEQ ID NO: 70)
VELIITFQLVFTI
(SEQ ID NO: 71)
LVELIITFQLVFTI

AQP4 region 171-185:

(SEQ ID NO: 72)
IFAS[CS]DSKR
(SEQ ID NO: 73)
TIFAS[CS]DSKR
(SEQ ID NO: 74)
FTIFAS[CS]DSKR
(SEQ ID NO: 75)
VFTIFAS[CS]DSKR
(SEQ ID NO: 76)
IFAS[CS]DSKRT
(SEQ ID NO: 77)
TIFAS[CS]DSKRT
(SEQ ID NO: 78)
FTIFAS[CS]DSKRT
(SEQ ID NO: 79)
VFTIFAS[CS]DSKRT
(SEQ ID NO: 80)
IFAS[CS]DSKRTD
(SEQ ID NO: 81)
TIFAS[CS]DSKRTD
(SEQ ID NO: 82)
FTIFAS[CS]DSKRTD
(SEQ ID NO: 83)
VFTIFAS[CS]DSKRTD
(SEQ ID NO: 84)
IFAS[CS]DSKRTDV
(SEQ ID NO: 85)
TIFAS[CS]DSKRTDV
(SEQ ID NO: 86)
FTIFAS[CS]DSKRTDV
(SEQ ID NO: 87)
VFTIFAS[CS]DSKRTDV

AQP4 region 202-216:

(SEQ ID NO: 88)
INYTGASMN
(SEQ ID NO: 89)
AINYTGASMN
(SEQ ID NO: 90)
FAINYTGASMN
(SEQ ID NO: 91)
LFAINYTGASMN
(SEQ ID NO: 92)
INYTGASMNP
(SEQ ID NO: 93)
AINYTGASMNP
(SEQ ID NO: 94)
FAINYTGASMNP
(SEQ ID NO: 95)
LFAINYTGASMNP
(SEQ ID NO: 96)
INYTGASMNPA
(SEQ ID NO: 97)
AINYTGASMNPA
(SEQ ID NO: 98)
FAINYTGASMNPA
(SEQ ID NO: 99)
LFAINYTGASMNPA
(SEQ ID NO: 100)
INYTGASMNPAR
(SEQ ID NO: 101)
AINYTGASMNPAR
(SEQ ID NO: 102)
FAINYTGASMNPAR
(SEQ ID NO: 103)
LFAINYTGASMNPAR

AQP4 region 249-263:

(SEQ ID NO: 104)
F[CS]PDVEFKR
(SEQ ID NO: 105)
VF[CS]PDVEFKR
(SEQ ID NO: 106)
YVF[CS]PDVEFKR
(SEQ ID NO: 107)
EYVF[CS]PDVEFKR
(SEQ ID NO: 108)
F[CS]PDVEFKRR
(SEQ ID NO: 109)
VF[CS]PDVEFKRR
(SEQ ID NO: 110)
YVF[CS]PDVEFKRR
(SEQ ID NO: 111)
EYVF[CS]PDVEFKRR
(SEQ ID NO: 112)
F[CS]PDVEFKRRF
(SEQ ID NO: 113)
VF[CS]PDVEFKRRF
(SEQ ID NO: 114)
YVF[CS]PDVEFKRRF
(SEQ ID NO: 115)
EYVF[CS]PDVEFKRRF
(SEQ ID NO: 116)
F[CS]PDVEFKRRFK
(SEQ ID NO: 117)
VF[CS]PDVEFKRRFK
(SEQ ID NO: 118)
YVF[CS]PDVEFKRRFK
(SEQ ID NO: 119)
EYVF[CS]PDVEFKRRFK

AQP4 region 284-298:

(SEQ ID NO: 120)
VETDDLILK
(SEQ ID NO: 121)
QVETDDLILK
(SEQ ID NO: 122)
SQVETDDLILK
(SEQ ID NO: 123)
RSQVETDDLILK
(SEQ ID NO: 124)
VETDDLILKP
(SEQ ID NO: 125)
QVETDDLILKP
(SEQ ID NO: 126)
SQVETDDLILKP
(SEQ ID NO: 127)
RSQVETDDLILKP
(SEQ ID NO: 128)
VETDDLILKPG
(SEQ ID NO: 129)
QVETDDLILKPG
(SEQ ID NO: 130)
SQVETDDLILKPG
(SEQ ID NO: 131)
RSQVETDDLILKPG
(SEQ ID NO: 132)
VETDDLILKPGV
(SEQ ID NO: 133)
QVETDDLILKPGV
(SEQ ID NO: 134)
SQVETDDLILKPGV
(SEQ ID NO: 135)
RSQVETDDLILKPGV,

Wherein, when present in any one of these epitope sequences, the residue [CS] stands for a single cysteine (C) or a single serine (S) residue.

Aspect 2. The peptide according to aspect 1, wherein the linker-epitope sequence is selected from:

(SEQ ID NO: 171)
RENIMVAFKGVW
(SEQ ID NO: 172)
KPLPVDMVLIS
(SEQ ID NO: 173)
TVAMVSTRKISI
(SEQ ID NO: 174)
AVTVAMVSTRKISI
(SEQ ID NO: 175)
AVTVAMVCTRKISI
(SEQ ID NO: 176)
TRKISIAKSVFY
(SEQ ID NO: 177)
LVELIITFQLVF
(SEQ ID NO: 178)
VFTIFASSDSKR
(SEQ ID NO: 179)
VFTIFASCDSKR
(SEQ ID NO: 180)
LFAINYTGASMN
(SEQ ID NO: 181)
EYVFSPDVEFKR
(SEQ ID NO: 182)
EYVFCPDVEFKR
(SEQ ID NO: 183)
RSQVETDDLILK

Aspect 3. The peptide according to aspect 1, wherein the epitope-flanker sequence is selected from:

(SEQ ID NO: 184)
IMVAFKGVWTQAK
(SEQ ID NO: 185)
LPVDMVLISLSFK
(SEQ ID NO: 186)
MVSTRKISIAK
(SEQ ID NO: 187)
MVSTRKISIAKKK
(SEQ ID NO: 188)
MVCTRKISIAKKK
(SEQ ID NO: 189)
MVCTRKISIAKK
(SEQ ID NO: 190)
ISIAKSVFYIAA
(SEQ ID NO: 191)
ISIAKSVFYIAAKK
(SEQ ID NO: 192)
ISIAKSVFYIAAKKK
(SEQ ID NO: 193)
LIITFQLVFTIDD
(SEQ ID NO: 194)
LIITFQLVFTIK
(SEQ ID NO: 195)
IFASSDSKRIDVK
(SEQ ID NO: 196)
IFASCDSKRIDVK
(SEQ ID NO: 197)
IFASCDSKRTDVKK
(SEQ ID NO: 198)
IFASCDSKRTDVK
(SEQ ID NO: 199)
INYTGASMNPAR
(SEQ ID NO: 200)
FSPDVEFKRRFK
(SEQ ID NO: 201)
FCPDVEFKRRFK
(SEQ ID NO: 202)
VETDDLILKPGVK

Aspect 4. The peptide according to aspect 1, wherein the linker-epitope-flanker sequence is selected from:

(SEQ ID NO: 203)
RENIMVAFKGVWTQAK
(SEQ ID NO: 204)
KPLPVDMVLISLSFK
(SEQ ID NO: 205)
TVAMVSTRKISIAK
(SEQ ID NO: 206)
AVTVAMVSTRKISIAKKK
(SEQ ID NO: 207)
AVTVAMVCTRKISIAKKK
(SEQ ID NO: 208)
AVTVAMVCTRKISIAKK
(SEQ ID NO: 209)
TRKISIAKSVFYIAA
(SEQ ID NO: 210)
TRKISIAKSVFYIAAKK
(SEQ ID NO: 211)
TRKISIAKSVFYIAAKKK
(SEQ ID NO: 212)
LVELIITFQLVFTIDD
(SEQ ID NO: 213)
LVELIITFQLVFTIK
(SEQ ID NO: 214)
VFTIFASSDSKRIDVK
(SEQ ID NO: 215)
VFTIFASCDSKRTDVK
(SEQ ID NO: 216)
VFTIFASCDSKRTDVKK
(SEQ ID NO: 217)
VFTIFASCDSKRTDVK
(SEQ ID NO: 218)
LFAINYTGASMNPAR
(SEQ ID NO: 219)
EYVFSPDVEFKRRFK
(SEQ ID NO: 220)
EYVFCPDVEFKRRFK
(SEQ ID NO: 221)
RSQVETDDLILKPGVK

In a preferred embodiment of aspect 4, said linker-epitope-flanker is selected from the group consisting of: TRKISIAKSVFYIAA, TRKISIAKSVFYIAAKK and TRKISIAKSVFYIAAKKK.

In a further preferred embodiment of aspect 4, said linker-epitope is selected from the group consisting of: EYVFSPDVEFKRRFK and EYVFCPDVEFKRRFK.

Aspect 5. The peptide according to any one of aspects 1 to 4, wherein said oxidoreductase motif is selected from the following amino acid motifs:

Zₘ-[CST]-Xₙ-C- or Zₘ-C-Xₙ-[CST]-, (a)
wherein n is 0, and wherein m is an integer selected from 0, 1, or 2,
wherein Z is any amino acid, preferably a basic amino acid preferably selected from: H, K, R, and a non-natural basic amino acid as defined herein, such as L-ornithine, more preferably K or H, most preferably K;

Zₘ-[CST]-Xₙ-C- or Zₘ-C-Xₙ-[CST]-, (b)
wherein n is 1, wherein X is any amino acid, preferably a basic amino acid selected from: H, K, R, and a non-natural basic amino acid such as L-ornithine, more preferably Kor R,
wherein m is an integer selected from 0, 1, or 2,
wherein Z is any amino acid, preferably a basic amino acid preferably selected from: H, K, R, and a non-natural basic amino acid as defined herein, such as L-ornithine, more preferably K or H, most preferably K;
(c) Zₘ-[CST]-Xₙ-C- or Zₘ-C-Xₙ-[CST]-, wherein n is 2, thereby creating an internal X1X2 amino acid couple within the oxidoreductase motif, wherein X is any amino acid, preferably wherein at least one X is a basic amino acid selected from: H, K, R, and a non-natural basic amino acid such as L-ornithine, more preferably K or R,
wherein m is an integer selected from 0, 1, or 2,
wherein Z is any amino acid, preferably a basic amino acid preferably selected from: H, K, R, and a non-natural basic amino acid as defined herein, such as L-ornithine, more preferably K or H, most preferably H;
(d) Zₘ-[CST]-Xₙ-C- or Zₘ-C-Xₙ-[CST]-, wherein n is 3, thereby creating an internal X¹X²X³ amino acid stretch within the oxidoreductase motif, wherein X is any amino acid, preferably wherein at least one X is a basic amino acid selected from: H, K, R, and a non-natural basic amino acid such as L-ornithine, more preferably K or R,
wherein m is an integer selected from 0, 1, or 2,
wherein Z is any amino acid, preferably a basic amino acid preferably selected from: H, K, R, and a non-natural basic amino acid as defined herein, such as L-ornithine, more preferably K or H, most preferably K;
(e) Zₘ-[CST]-Xₙ-C- or Zₘ-C-Xₙ-[CST]-, wherein n is 4, thereby creating an internal X¹X²X³X⁴ amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K;
(f) Zₘ-[CST]-Xₙ-C- or Zₘ-C-Xₙ-[CST]-, wherein n is 5, thereby creating an internal X¹X²X³X⁴X⁵ amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K;
(g) Zₘ-[CST]-Xₙ-C- or Zₘ-C-Xₙ-[CST]-, wherein n is 6, thereby creating an internal X¹X²X³X⁴X⁵X⁶ amino acid stretch within the oxidoreductase motif, wherein m is an integer selected from 0, 1, or 2, wherein Z is any amino acid, preferably a basic amino acid selected from: H, K, R, and a non-natural basic amino acid as defined herein, such as L-ornithine, preferably K or H, most preferably K; or
(h) Zₘ-[CST]-Xₙ-C- or Zₘ-C-Xₙ-[CST]-, wherein n is 0 to 6 and wherein m is 0, and wherein one of the C or [CST] residues has been modified so as to carry an acetyl, methyl, ethyl or propionyl group, either on the N-terminal amide of the amino acid residue of the motif or on the C-terminal carboxy group.

Aspect 6. The peptide according to any one of aspects 1 to 5, wherein the oxidoreductase motif has the following general sequence formula:

Zₘ-[CST]-XX-C- or Zₘ-C-XX-[CST]-.

Aspect 7. The peptide according to any one of aspects 1 to 6, wherein the Xn or the XX portion of said oxidoreductase motif comprises the sequence PY or GH.

Aspect 8. The peptide according to any one of aspects 1 to 7, wherein amino acid Z of the oxidoreductase motif is a basic amino acid, preferably a basic amino acid selected from the group of amino acids consisting of: H, K, R, and any non-natural basic amino acid, more preferably a basic amino acid selected from: H, K, and R, most preferably wherein Z is H or K.

Aspect 9. The peptide according to any one of aspects 1 to 8, wherein the oxidoreductase motif is identified by any one of the following sequences: CPYC (SEQ ID NO: 157), HCPYC (SEQ ID NO: 158), KHCPYC (SEQ ID NO: 159), KCPYC (SEQ ID NO: 160), RCPYC (SEQ ID NO: 161), KKCPYC (SEQ ID NO: 162), KRCPYC (SEQ ID NO: 163), CHGC (SEQ ID NO: 164), HCGHC (SEQ ID NO: 165), KCGHC (SEQ ID NO: 166), KHCGHC (SEQ ID NO: 167), RCGHC (SEQ ID NO: 168), KKCGHC (SEQ ID NO: 169), and KRCGHC (SEQ ID NO: 170), more preferably HCPYC (SEQ ID NO: 158) or KHCPYC (SEQ ID NO: 159).

Alternative examples of such motifs are: KCC, KKCC (SEQ ID NO: 231), RCC, RRCC (SEQ ID NO: 232), RKCC (SEQ ID NO: 233), or KRCC (SEQ ID NO: 234), KCXC (SEQ ID NO: 235), KKCXC (SEQ ID NO: 242), RCXC (SEQ ID NO: 237), RRCXC (SEQ ID NO: 238), RKCXC (SEQ ID NO: 247), KRCXC (SEQ ID NO: 248), KCKC (SEQ ID NO: 235), KKCKC (SEQ ID NO: 242), KCRC (SEQ ID NO: 243), KKCRC (SEQ ID NO: 244), RCRC (SEQ ID NO: 245), RRCRC (SEQ ID NO: 246), RKCKC (SEQ ID NO: 247), KRCKC (SEQ ID NO: 248), or RCKC (SEQ ID NO: 249), more preferably KCRC (SEQ ID NO: 243).

Further alternative examples of such motifs are: CRPYC (SEQ ID NO: 250), KCRPYC (SEQ ID NO: 251), KHCRPYC (SEQ ID NO: 252), RCRPYC (SEQ ID NO: 253), HCRPYC (SEQ ID NO: 254), CPRYC (SEQ ID NO: 255), KCPRYC (SEQ ID NO: 256), RCPRYC (SEQ ID NO: 257), HCPRYC (SEQ ID NO: 258), CPYRC (SEQ ID NO: 259), KCPYRC (SEQ ID NO: 260), RCPYRC (SEQ ID NO: 261), HCPYRC (SEQ ID NO: 262), CKPYC (SEQ ID NO: 263), KCKPYC (SEQ ID NO: 264), RCKPYC (SEQ ID NO: 265), HCKPYC (SEQ ID NO: 266), CPKYC (SEQ ID NO: 267), KCPKYC (SEQ ID NO: 268), RCPKYC (SEQ ID NO: 269), HCPKYC (SEQ ID NO: 270), CPYKC (SEQ ID NO: 271), KCPYKC (SEQ ID NO: 272), RCPYKC (SEQ ID NO: 273), and HCPYKC (SEQ ID NOs: 274), more preferably KCRPYC (SEQ ID NO: 251).

In a specific embodiment of aspect 9, said motif is selected from the group consisting of: HCPYC and KHCPYC.

In a further specific embodiment of aspect 9, said motif is selected from the group consisting of: KCRC and KCRPYC.

Aspect 10. The peptide according to any one of aspects 1 to 9, wherein said peptide comprises or consists of the amino sequences depicted in SEQ ID NO's 137 to 156:

(SEQ ID NO: 137)
HCPYCRENIMVAFKGVWTQAK
(SEQ ID NO: 138)
KHCPYCKPLPVDMVLISLSFK
(SEQ ID NO: 139)
HCPYCTVAMVSTRKISIAK
(SEQ ID NO: 140)
KHCPYCAVTVAMVSTRKISIAKKK
(SEQ ID NO: 141)
KHCPYCAVTVAMVCTRKISIAKKK
(SEQ ID NO: 142)
HCPYCAVTVAMVCTRKISIAKK
(SEQ ID NO: 143)
HCPYCTRKISIAKSVFYIAA
(SEQ ID NO: 144)
HCPYCTRKISIAKSVFYIAAKK
(SEQ ID NO: 145)
KHCPYCTRKSIAKSVFYIAAKK
(SEQ ID NO: 146)
HCPYCTRKISIAKSVFYIAAKKK
(SEQ ID NO: 147)
HCPYCLVELIITFQLVFTIDD
(SEQ ID NO: 148)
KHCPYCLVELIITFOLVFTIK
(SEQ ID NO: 149)
HCPYCVETIFASSDSKRTDVK
(SEQ ID NO: 150)
HCPYCVETIFASCDSKRTDVK
(SEQ ID NO: 151)
HCPYCVFTIFASCDSKRTDVKK
(SEQ ID NO: 152)
KHCPYCVFTIFASCDSKRTDVK
(SEQ ID NO: 153)
KHCPYCLFAINYTGASMNPAR
(SEQ ID NO: 154)
HCPYCEYVFSPDVEFKRRFK
(SEQ ID NO: 155)
HCPYCEYVFCPDVEFKRRFK
or
(SEQ ID NO: 156)
HCPYCRSQVETDDLILKPGVK

In a specific embodiment, said immunogenic peptide has the following sequence: HCPYCTRKISIAKSVFYIAAKKK (also called P12) or HCPYCEYVFSPDVEFKRRFK (also called P20).

Aspect 11. The immunogenic peptide according to any one of aspects 1 to 10, wherein said T cell epitope is an NKT cell epitope and the peptide has a length of between 12 and 50 amino acids, preferably of between 12 and 30 amino acids; or wherein said T-cell epitope is an MHC class II T cell epitope and the peptide has a length of between 12 and 50 amino acids, preferably of between 12 and 30 amino acids.

Aspect 12. A polynucleotide (nucleic acid molecule) encoding the immunogenic peptide according to any one of aspects 1 to 11, preferably selected from isolated desoxyribonucleic acid (DNA), plasmid DNA (pDNA), coding DNA (cDNA), ribonucleic acid (RNA), messenger RNA (mRNA) or modified versions thereof, such as non-immunogenic mRNA comprising N(1)-methyl-pseudouridine (m1w). In some embodiments, said nucleic acid can be part of an expression cassette, optionally incorporated in a (viral) vector or plasmid that can be used for gene-therapy or can be present in the form of encapsulated or naked DNA or RNA to be administered according to techniques known in the pharmaceutical and gene therapeutic field.

Aspect 13. The peptide according to any one of aspects 1 to 11, or the polynucleotide according to aspect 12, for use as a medicament.

Aspect 14. The peptide or polynucleotide according to aspect 13 for use in the treatment of, for ameliorating the symptoms of, or for preventing an anti-AQP4 disease or a Neuromyelitis Optica Spectrum Disorder. Preferred are diseases or disorders caused or aggravated by AQP4 auto-antigens and/or anti-AQP4 antibodies. Such diseases or disorders include but are not limited to: NMO; Optic Neuritis; Devic's disease; AQP4-positive Optic-Spinal MS (OSMS); Longitudinally Extensive (Transverse) Myelitis; AQP4-positive myelitis, preferably associated with lesions in specific brain areas such as the hypothalamus, periventricular nucleus, and brainstem; and Tumefactive demyelination or lesions.

In a preferred embodiment, said treatment is combined with, e.g. simultaneously, sequentially or separately, an antibody depletion therapy as defined herein. In a preferred embodiment, such antibody depletion treatment precedes the immunogenic peptide treatment.

Aspect 15. An in vitro method for the generation of a population of cytolytic CD4+ T cells, against APC presenting AQP4 epitopes, comprising the steps of:
providing peripheral blood cells;
contacting said cells in vitro with the peptide of any one of aspects 1 to 11, or the polynucleotide according to aspect 12; and
expanding said cells in the presence of IL-2.

Aspect 16. A method for the generation of a population of cytolytic CD4+ T cells, against APC presenting AQP4 epitopes, comprising the steps of:
administering to a subject an effective amount of the peptide of any one of aspects 1 to 11, or the polynucleotide according to aspect 12;
obtaining said cytolytic CD4+ T cells from a peripheral blood cell population of said subject.

Aspect 17. A method for the generation of a population of NKT cells, against APC presenting AQP4 epitopes, comprising the steps of:
administering to a subject an effective amount of the peptide of any one of aspects 1 to 11, or the polynucleotide according to aspect 12;
obtaining said NKT cells from a peripheral blood cell population of said subject.

Aspect 18. A population of cytolytic CD4+ T cells or NKT cells, against APC presenting AQP4 epitopes, obtainable by the method of aspect 15, 16 or 17.

Aspect 19. A population of cytolytic CD4+ T cells or NKT cells, against APC presenting AQP4 epitopes, obtainable by the method of aspect 15, 16 or 17, for use as a medicament.

Aspect 20. A population of cytolytic CD4+ T cells or NKT cells for use according to aspect 19, for use in the treatment of, ameliorating the symptoms of, and/or preventing of an anti-AQP4 disease or a Neuromyelitis Optica Spectrum Disorder. Preferred disorders include but are not limited to: NMO; Optic Neuritis; Devic's disease; AQP4-positive Optic-Spinal MS (OSMS); Longitudinally Extensive (Transverse) Myelitis; AQP4-positive myelitis, preferably associated with lesions in specific brain areas such as the hypothalamus, periventricular nucleus, and brainstem; and Tumefactive demyelination or lesions.

In a preferred embodiment, said treatment is combined with, e.g. simultaneously, sequentially or separately, an antibody depletion therapy as defined herein. In a preferred embodiment, such antibody depletion treatment precedes the immunogenic peptide treatment.

Aspect 21. A pharmaceutical composition comprising the peptide of any one of aspects 1 to 11, the polynucleotide according to aspect 12, or the CD4+ T cells or NKT cells of any one of aspects 18 to 20, or any mixture thereof.

Aspect 22. The pharmaceutical composition of aspect 21, optionally further comprising a pharmaceutically acceptable carrier, and optionally further comprising an additional active ingredient suitable for the treatment of, for ameliorating the symptoms of, or for preventing an anti-AQP4 disease or a Neuromyelitis Optica Spectrum Disorder. Preferred are diseases or disorders caused or aggravated by AQP4 auto-antigens and/or anti-AQP4 antibodies. Such diseases or disorders include but are not limited to: NMO; Optic Neuritis; Devic's disease; AQP4-positive Optic-Spinal MS (OSMS); Longitudinally Extensive (Transverse) Myelitis; AQP4-positive myelitis, preferably associated with lesions in specific brain areas such as the hypothalamus, periventricular nucleus, and brainstem; and Tumefactive demyelination or lesions.

Aspect 23. The pharmaceutical composition of aspect 21 or 22, for use as a medicament.

Aspect 24. The pharmaceutical composition for use according to aspect 23, for use in the treatment of, for ameliorating the symptoms of, or for preventing an anti-AQP4 disease or a Neuromyelitis Optica Spectrum Disorder. Preferred are diseases or disorders caused or aggravated by AQP4 auto-antigens and/or anti-AQP4 antibodies. Such diseases or disorders include but are not limited to: NMO; Optic Neuritis; Devic's disease; AQP4-positive Optic-Spinal MS (OSMS); Longitudinally Extensive (Transverse) Myelitis; AQP4-positive myelitis, preferably associated with lesions in specific brain areas such as the hypothalamus, periventricular nucleus, and brainstem; and Tumefactive demyelination or lesions.

In a preferred embodiment, said treatment is combined with, e.g. simultaneously, sequentially or separately, an antibody depletion therapy as defined herein. In a preferred embodiment, such antibody depletion treatment precedes the immunogenic peptide treatment.

Aspect 25. The peptide, polynucleotide, CD4+ T cells, NKT cells, or pharmaceutical composition according to any one of the previous aspects for use in treating of, for ameliorating the symptoms of, and/or for preventing of Neuromyelitis Optica (NMO).

In a preferred embodiment, said treatment is combined with, e.g. simultaneously, sequentially or separately, an antibody depletion therapy as defined herein. In a preferred embodiment, such antibody depletion treatment precedes the immunogenic peptide treatment.

Aspect 26. The peptide, polynucleotide, CD4+ T cells, NKT cells, or pharmaceutical composition for use in treating of, ameliorating the symptoms of, and/or preventing NMO, according to any one of the previous aspects, wherein the subject has an HLA type selected from the group consisting of: HLA-DRB1*03:01 and HLA-DPB1*05:01, preferably HLA-DRB1*03:01.

Aspect 27. Use of an immunogenic peptide according to any one of aspects 1 to 11, the polynucleotide according to aspect 12, or the CD4+ T cells or NKT cells of any one of aspects 18 to 20, or any mixture thereof, for the manufacture of a medicament for treating of, ameliorating the symptoms of, and/or preventing of a Neuromyelitis Optica Spectrum Disorder, preferably a disorder caused or aggravated by AQP4 auto-antigens and/or anti-AQP4 antibodies, most preferably Neuromyelitis Optica (NMO).

In a preferred embodiment, said treatment is combined with, e.g. simultaneously, sequentially or separately, an antibody depletion therapy as defined herein. In a preferred embodiment, such antibody depletion treatment precedes the immunogenic peptide treatment.

Aspect 28. A method for treating of, ameliorating the symptoms of, and/or preventing a Neuromyelitis Optica Spectrum Disorder in a subject, comprising the step of administering a therapeutically effective amount of the peptide according to aspects 1 to 11, the polynucleotide according to aspect 12, or the CD4+ T cells or NKT cells of any one of aspects 18 to 20, or any mixture thereof, to a subject.

In a preferred embodiment, said treatment is combined with, e.g. simultaneously, sequentially or separately, an antibody depletion therapy as defined herein. In a preferred embodiment, such antibody depletion treatment precedes the immunogenic peptide treatment.

Aspect 29. The method according to aspect 28, wherein said anti-AQP4 disease or Neuromyelitis Optica Spectrum Disorder is a disease or disorder caused or aggravated by AQP4 auto-antigens and/or anti-AQP4 antibodies. Such diseases or disorders include but are not limited to: NMO; Optic Neuritis; Devic's disease; AQP4-positive Optic-Spinal MS (OSMS); Longitudinally Extensive (Transverse) Myelitis; AQP4-positive myelitis, preferably associated with lesions in specific brain areas such as the hypothalamus, periventricular nucleus, and brainstem; and Tumefactive demyelination or lesions.

In a preferred embodiment, said treatment is combined with, e.g. simultaneously, sequentially or separately, an antibody depletion therapy as defined herein. In a preferred embodiment, such antibody depletion treatment precedes the immunogenic peptide treatment.

Aspect 30. An in vitro method for detecting MHC class Il restricted CD4+ T cells specific for a AQP4 antigen in a sample comprising the steps of;
contacting a subject sample with a complex of an isolated MHC class Il molecules and a peptide according to aspects 1 to 11;
detecting CD4+ T cells by measuring the binding of said complex with cells in said sample, wherein the binding of the complex to a cell is indicative for the presence of CD4+ T cells specific for a AQP4 antigen in said sample.

Aspect 31. A method for treating of, ameliorating the symptoms of, and/or preventing a Neuromyelitis Optica Spectrum Disorder in a subject, comprising the step of administering to a subject a therapeutically effective amount of the peptide according to any one of aspects 1 to 11, the polynucleotide according to aspect 12, or the CD4+ T cells or NKT cells of any one of aspects 18 to 20, and an antibody having B cell depleting activity, wherein said antibody and said immunogenic peptide, polynucleotide or cells are administered either simultaneously, sequentially or separately.

Aspect 32. The method according to aspect 31, wherein said antibody having B cell depleting activity is administrated before said immunogenic peptide, polynucleotide or cells.

Aspect 33. The method according to aspect 31 or 32, wherein said antibody having B cell depleting activity is selected from one that binds an antigen selected from the group consisting of CD10, CD19, CD20, CD21, CD22, CD23, CD24, CD37, CD53, CD72, CD73, CD74, CDw75, CDw76, CD77, CDw78, CD79a, CD79b, CD80 (B7.1), CD81, CD82, CD83, CDw84, CD85 and CD86 (B7.2).

In a preferred embodiment, said antibody having B cell depleting activity is selected from one that binds CD19, such as Inebilizumab (MEDI-551).

In another preferred embodiment, said antibody having B cell depleting activity is selected from one that binds CD20, such as Rituximab or Ublituximab (LFB-R603, TGT-1101, TGTX-1101).

Aspect 34. A pharmaceutical preparation (combination or pharmaceutical composition or kit-of-parts) comprising the peptide according to aspects 1 to 11, the polynucleotide according to aspect 12, or the CD4+ T cells or NKT cells of any one of aspects 18 to 20, and an antibody having B cell depleting activity.

Aspect 35. The pharmaceutical preparation according to aspect 34, wherein said antibody having B cell depleting activity is selected from one that binds an antigen selected from the group consisting of CD10, CD19, CD20, CD21, CD22, CD23, CD24, CD37, CD53, CD72, CD73, CD74, CDw75, CDw76, CD77, CDw78, CD79a, CD79b, CD80 (B7.1), CD81, CD82, CD83, CDw84, CD85 and CD86 (B7.2).

In a preferred embodiment, said antibody having B cell depleting activity is selected from one that binds CD19, such as Inebilizumab (MEDI-551).

In another preferred embodiment, said antibody having B cell depleting activity is selected from one that binds CD20, such as Rituximab or Ublituximab (LFB-R603, TGT-1101, TGTX-1101).

Aspect 36. A pharmaceutical preparation according to any one of aspects 34 to 35, for use as a medicament.

Aspect 37. A pharmaceutical preparation according to any one of aspects 34 to 35, for use in treating, ameliorating the symptoms, and/or preventing a Neuromyelitis Optica Spectrum Disorder.

Aspect 38. The pharmaceutical preparation for use according to aspect 36 or 37, wherein said antibody and said immunogenic peptide, polynucleotide or cells are administered either simultaneously, sequentially or separately.

Aspect 39. The pharmaceutical preparation for use according to aspect 38, wherein said antibody having B cell depleting activity is administrated before said immunogenic peptide, polynucleotide or cells.

The above and further aspects and preferred embodiments of the invention are described in the following sections and in the appended claims. The subject matter of the appended claims is hereby specifically incorporated in this specification.

### BRIEF DESCRIPTION OF THE DRAWINGS

FIGS. 1A-1E: Kinetics of the redox activities of the peptides P12 and P20 (FIG. 1A), P1, P3, P7 and P15 (FIG. 1B), P4, P11 and P22 (FIG. 1C), P5, P6, P16 and

P21 (FIG. 1D), and P10, P17 and P18 (FIG. 1E). DTT is used as a positive control, while Blank represents the assay buffer. The results are expressed in Relative Fluorescent Units (RFU). The assay is described in detail in the Examples section.

FIGS. 2A-2E: HLA-DR3 (DRB1*03:01) binding of the peptides P12 and P20 (FIG. 2A), P10 and P21 (FIG. 2B), P15 and P18 (FIG. 2C), P4 and P6 (FIG. 2D), and P1, P3 and P7 (FIG. 2E). The decreasing fluorescence signal (RFU) demonstrates the dose-dependent relationship produced following the competition with biotin-tagged high-affinity control peptide and revealed by Eu3+streptavidin interaction.

FIG. 3: Frequency of effector cells (CD154+) specific for the P20 peptide in the CD4 +T cell-lines of patients NMO-001 (S8), NMO-003 (S11) (S, number of stimulations with P20).

FIG. 4: Frequency of effector cells (CD154+) specific for the P20 peptide and its corresponding short-S-WT epitope (AGGLYEYVFSPDVEFKRRFK, SEQ ID NO: 397) on the CD4+ T cell-line of patient NMO-001 (S14) (S, number of stimulations with P20).

FIG. 5: Specific secretion of cytokines (IL-5 and IL-13) induced by P20 peptide and its corresponding short-C-WT epitope (AGGLYEYVFCPDVEFKRRFK, SEQ ID NO: 398) at two doses, in culture supernatant of NMO-006 CD4+ T cell-line (S11) (S, number of stimulations with P20).

FIG. 6: Percentage of specific LCL apoptosis when labelled autologous LCL, loaded with P20 peptide, its corresponding short-S-WT epitope (AGGLYEYVFSPDVEFKRRFK, SEQ ID NO: 397) or P10 irrelevant peptide, are co-cultured with the P20-specific CD4+ T cell-lines of patients NMO-001 (S14), NMO-003 (S14) (S, number of stimulations with P20; ND, not determined).

FIG. 7: Percentage of specific LCL apoptosis when labelled autologous LCL, loaded with P20 peptide at two doses, its corresponding short-C-WT epitope (AGGLYEYVFCPDVEFKRRFK, SEQ ID NO: 398) at two doses or P10 irrelevant peptide, are co-cultured with the P20-specific CD4+ T cell-lines of patients NMO-001 (S16) (S, number of stimulations with P20).

FIG. 8: Secretion of the lytic marker Granzyme B (GZMB) induced by P20, its corresponding short-S-WT epitope (AGGLYEYVFSPDVEFKRRFK, SEQ ID NO: 397) or P10 irrelevant peptide, in culture supernatant of NMO-001 and NMO-003 CD4+ T cell-lines (S14) (S, number of stimulations with P20; ND, not determined).

FIG. 9: Secretion of the lytic marker Granzyme B (GZMB) induced by P20, its corresponding short-C-WT epitope (AGGLYEYVFCPDVEFKRRFK, SEQ ID NO: 398) at two doses, in culture supernatant of NMO-006 CD4+ T cell-line (S11) (S, number of stimulations with P20).

FIG. 10: Frequency of effector cells (CD154+) specific for the P12 peptide on the CD4+ T cell-lines of patients NMO-001 (S7), NMO-003 (S8) (S, number of stimulations with P12).

FIG. 11: Frequency of effector cells (CD154+) specific for the P12 peptide and its corresponding short-WT epitope (KVAMVCTRKISIAKSVFYIAAKK, SEQ ID NO: 399) on the CD4+ T cell-line of patient NMO-001 (S12) (S, number of stimulations with P12).

FIG. 12: Specific secretion of cytokines (IL-5 and IL-13) induced by P12 in culture supernatant of NMO-003 CD4+ T cell-line (S8) (S, number of stimulations with P12).

FIG. 13: Percentage of specific LCL apoptosis when labelled autologous LCL, loaded P12 peptide, its corresponding short-WT epitope with (KVAMVCTRKISIAKSVFYIAAKK, SEQ ID NO: 399) or P20 irrelevant peptide, are co-cultured with the P12-specific CD4+ T cell-line of patient NMO-001 (S13) (S, number of stimulations with P12).

FIG. 14: Secretion of the lytic marker Granzyme B (GZMB) induced by P12, its corresponding short-WT epitope (KVAMVCTRKISIAKSVFYIAAKK, SEQ ID NO: 399) or P20 irrelevant peptide, in culture supernatant of NMO-001 CD4+ T cell-line (S13) (S, number of stimulations with P12).

### DETAILED DESCRIPTION OF THE INVENTION

The present invention will be described with respect to particular embodiments, but the invention is not limited thereto but only by the claims. Any reference signs in the claims shall not be construed as limiting the scope. The following terms or definitions are provided solely to aid in the understanding of the invention. Unless specifically defined herein, all terms used herein have the same meaning as they would to one skilled in the art of the present invention. The definitions provided herein should not be construed to have a scope less than the one understood by a person of ordinary skill in the art. Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise. The term “any” when used in relation to aspects, claims or embodiments as used herein refers to any single one (i.e. anyone) as well as to all combinations of said aspects, claims or embodiments referred to.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. Said terms also encompass the embodiments “consisting essentially of” and “consisting of”.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

As used herein, the term “for use” as used in “composition for use in treatment of a disease” shall disclose also the corresponding method of treatment and the corresponding use of a preparation for the manufacture of a medicament for the treatment of a disease”.

The term “peptide” as used herein refers to a molecule comprising an amino acid sequence of between 9 and 200 amino acids, connected by peptide bonds, but which can comprise non-amino acid structures, synthetic amino acids or modified amino acids.

Peptides according to the invention can contain proteinogenic and/or non-proteinogenic amino acids. Said peptides can contain any of the conventional 20 amino acids or modified versions thereof, or can contain non-naturally occurring amino-acids incorporated by chemical peptide synthesis or by chemical or enzymatic modification.

The term “antigen” as used herein refers to a structure of a macromolecule, typically protein (with or without polysaccharides) or made of proteic composition comprising one or more hapten(s) and comprising T cell epitopes.

The term “antigenic protein” as used herein refers to a protein comprising one or more T cell epitopes. An auto-antigen or auto-antigenic protein as used herein refers to a human or animal protein present in the body, which elicits an immune response within the same human or animal body.

The term “epitope” refers to one or several portions (which may define a conformational epitope) of an antigenic protein which is/are specifically recognised and bound by an antibody or a portion thereof (Fab′, Fab2′, etc.) or a receptor presented at the cell surface of a B or T cell lymphocyte, and which is able, by said binding, to induce an immune response.

The term “T cell epitope” in the context of the present invention refers to a dominant, sub-dominant or minor T cell epitope, i.e. a part of an antigenic protein that is specifically recognised and bound by a receptor at the cell surface of a T or NKT cell. Whether an epitope is dominant, sub-dominant or minor depends on the immune reaction elicited against the epitope. Dominance depends on the frequency at which such epitopes are recognised by T or NKT cells and able to activate them, among all the possible T cell epitopes of a protein.

In the context of the present invention, the T cell epitope can be an epitope recognized by MHC class Il molecules and presented to CD4+ T cells, or can be an epitope recognized by CD1d molecules and presented to NKT cells.

An epitope recognised by MHC class Il molecules typically comprises or consists of a sequence of +/−9 amino acids which fit in the groove of the MHC II molecule. Within a peptide sequence representing a T cell epitope, the amino acids in the epitope are numbered P1 to P9, amino acids N-terminal of the epitope are numbered P-1, P-2 and so on, amino acids C terminal of the epitope are numbered P+1, P+2 and so on. Peptides recognised by MHC class Il molecules and not by MHC class I molecules are referred to as MHC class Il restricted T cell epitopes.

Identification of MHC class Il restricted T cells epitopes is well-known in the art. Typically, prediction software are used and allows to identify epitopes for the desire HLA molecule(s) within an antigen.

The programs that can be used are shown in Table A:

TABLE A
T-Epitope Prediction Tools.

| Tool | Link |
|---|---|
| EpiDOCK | http://www.ddg- |
| | pharmfac.net/epidock/EpiDockPage.html |
| IEDB | http://tools.immuneepitope.org/mhcii/ |
| NetMHCII 2.3 | http://www.cbs.dtu.dk/services/NetMHCII/ |
| EpiJen | http://www.ddg- |
| | pharmfac.net/epijen/EpiJen/EpiJen.htm |
| NetMHCIIpan 4.0 | http://www.cbs.dtu.dk/services/NetMHCIIpan/ |
| EpiTOP | http://www.ddg- |
| | pharmfac.net/EpiTOP3/index.html |
| ProPRED | http://crdd.osdd.net/raghava/propred/ |
| Rankpep | http://imed.med.ucm.es/Tools/rankpep.html |
| SYFPEITHI | http://www.syfpeithi.de/bin/MHCServer.dll/ |
| | EpitopePrediction.htm |
| MHCPred | http://www.ddg- |
| | pharmfac.net/mhcpred/MHCPred/ |
| MHC2Pred | http://crdd.osdd.net/raghava/mhc2pred/ |
| IMTECH | http://crdd.osdd.net/raghava/mhc/page4.html |

The selected epitopes are then screened by using property calculators (Table B), allowing to predict the physicochemical properties of the epitopes, such as molecular weight, isoelectric point, hydrophicity/hydrophilicity, net charge at pH 7, etc.

TABLE B
Peptide properties calculators.

| Tool | Link |
|---|---|
| Bachem Peptide | https://www.bachem.com/service- |
| Calculator | support/peptide-calculator/ |
| BioSunthesis | https://www.biosyn.com/peptidepropertycalculator/ |
| | peptidepropertycalculator.aspx |
| pi Peptide Tool | https://www.protpi.ch/Calculator/PeptideTool |
| Innovagen PepCalc | https://pepcalc.com/ |
| Peptide Library | https://www.sigmaaldrich.com/technical- |
| Design | documents/articles/biology/peptide-library- |
| | design-and-calculator-tool.html |
| Peptide Draw | http://pepdraw.com/ |

Selected epitopes are then tested for their HLA binding and CD4 T cell activation capabilities as described herein.

The term “MHC” refers to “major histocompatibility antigen”. In humans, the MHC genes are known as HLA (“human leukocyte antigen”) genes. Although there is no consistently followed convention, some literature uses HLA to refer to HLA protein molecules, and MHC to refer to the genes encoding the HLA proteins. As such the terms “MHC” and “HLA” are equivalents when used herein. The HLA system in man has its equivalent in the mouse, i.e., the H2 system. The most intensely-studied HLA genes are the nine so-called classical MHC genes: HLA-A, HLA-B, HLA-C, HLA-DPA1, HLA-DPB1, HLA-DQA1, HLAS DQB1, HLA-DRA, and HLA-DRB1. In humans, the MHC is divided into three regions: Class I, II, and III. The A, B, and C genes belong to MHC class I, whereas the six D genes belong to class II. MHC class I molecules are made of a single polymorphic chain containing 3 domains (alpha 1, 2 and 3), which associates with beta-2-microglobulin at cell surface. Class II molecules are made of 2 polymorphic chains, each containing 2 chains (alpha 1 and 2, and beta 1 and 2). Class I MHC molecules are expressed on virtually all nucleated cells. Since the HLA system is inherited in a Mendelian manner, HLA series of genes, or haplotypes, can be distinguished in subjects of a given population.

Preferred HLA haplotypes of NMO patients in the current invention are therefore HLA-DRB1*03:01 and HLA-DPB1*05:01, more preferably HLA-DRB1*03:01.

Peptide fragments presented in the context of class I MHC molecules are recognised by CD8+T lymphocytes (cytolytic T lymphocytes or CTLs). CD8+T lymphocytes frequently mature into cytolytic effectors which can lyse cells bearing the stimulating antigen. Class II MHC molecules are expressed primarily on activated lymphocytes and antigen-presenting cells. CD4+ T lymphocytes (helper T lymphocytes or Th) are activated with recognition of a unique peptide fragment presented by a class II MHC molecule, usually found on an antigen-presenting cell like a macrophage or dendritic cell. CD4+ T lymphocytes proliferate and secrete cytokines such as IL-2, IFN-gamma and IL-4 that support antibody-mediated and cell mediated responses.

Functional HLAs are characterised by a deep binding groove to which endogenous as well as foreign, potentially antigenic peptides bind. The groove is further characterised by a well-defined shape and physico-chemical properties. HLA class I binding sites are closed, in that the peptide termini are pinned down into the ends of the groove. They are also involved in a network of hydrogen bonds with conserved HLA residues. In view of these restraints, the length of bound peptides is limited to 8, 9 or 10 residues. However, it has been demonstrated that peptides of up to 12 amino acid residues are also capable of binding HLA class I. Comparison of the structures of different HLA complexes confirmed a general mode of binding wherein peptides adopt a relatively linear, extended conformation, or can involve central residues to bulge out of the groove.

In contrast to HLA class I binding sites, class Il sites are open at both ends. This allows peptides to extend from the actual region of binding, thereby “hanging out” at both ends. Class II HLAs can therefore bind peptide ligands of variable length, ranging from 7, 8 or 9 to more than 25 amino acid residues. Similar to HLA class I, the affinity of a class Il ligand is determined by a “constant” and a “variable” component. The constant part again results from a network of hydrogen bonds formed between conserved residues in the HLA class Il groove and the main chain of a bound peptide. However, this hydrogen bond pattern is not confined to the N-and C-terminal residues of the peptide but distributed over the whole chain. The latter is important because it restricts the conformation of complexed peptides to a strictly linear mode of binding. This is common for all class Il allotypes. The second component determining the binding affinity of a peptide is variable due to certain positions of polymorphism within class Il binding sites. Different allotypes form different complementary pockets within the groove, thereby accounting for subtype-dependent selection of peptides, or specificity. Importantly, the constraints on the amino acid residues held within class Il pockets are in general “softer” than for class I. There is much more cross reactivity of peptides among different HLA class Il allotypes. The sequence of the +/−9 amino acids (i.e. 8, 9 or 10) of an MHC class II T cell epitope that fit in the groove of the MHC II molecule are usually numbered P1 to P9. Additional amino acids N-terminal of the epitope are numbered P-1, P-2 and so on, amino acids C-terminal of the epitope are numbered P+1, P+2 and so on.

An epitope recognised by CD1d molecules refers to a part of an antigenic protein that is specifically recognized and bound by a receptor at the cell surface of a T lymphocyte, in particular NKT cells. An epitope recognised by CD1d molecules typically comprises or consists of a sequence of +/−7 amino acids which fit in the groove of the CD1d molecules. Typically, NKT cell epitopes are hydrophobic. The structure of the CD1d molecule indicates that hydrophobic amino acid residues are required to occupy the two hydrophobic pockets located at the extremities of the CD1d cleft and that an aliphatic residue should occupy the position in the middle of the cleft. Therefore, as a general but not limiting example of CD1d binding sequence, the motif [FWHY]-xx-[ILMV]-xx-[FWHY] (SEQ ID NO: 312) or [FW]-XX-[ILMV]-XX-[FW] (SEQ ID NO: 313) can be used in which [FWHY] indicates that either F, W, H or Y can occupy the first anchoring residue (P1), that the P4 position can be occupied by either I, L, M or V and that P7 can be occupied by F, W, H or Y. “x” in this general model motif stands for any amino acid. In a particular embodiment, the general model motif can be defined by the sequence [FW]-xx-[ILM]-xx-[FW] (SEQ ID NO: 318), preferably by the sequence [FW]-xx-[ILM]-xx-[W] (SEQ ID NO: 319). The term “NKT cells” refers to cells of the innate immune system characterized by the fact that they carry receptors such as NK1.1 and NKG2D, and recognize epitopes presented by the CD1d molecule. In the context of the present invention, NKT cells can belong to either the type 1 (invariant) or the type 2 subset, or to any of the less characterized NKT cells with more polymorphic T cell receptors than type 1 or type 2 NKT cells. The participation of NKT cells in the control of immune responses in auto-immune diseases, or against allofactors or allergens has been reported on a number of occasions (Jahng et al Journal of experimental Medicine 199: 947-957, 2004; Van Belle and von Herrath, Molecular Immunology 47: 8-1 1, 2009) but is relatively difficult to describe. In the context of the present invention, we made the unexpected observation that peptides can be presented by the CD1d molecule. A characteristic of the CD1d molecule is to be made of 2 anti-parallel alpha chains forming a cleft sitting atop of a platform made of two anti- parallel beta chains. The cleft is narrow and deep and accept only hydrophobic residues, classically deemed to be only lipids. In fact, peptides with hydrophobic residues have the capacity to bind to the CD1d cleft. Besides, as the cleft is open both sides, peptides longer than 7 aminoacids can be accommodated. Hydrophobic peptides carrying the CD1d motif are found in autoantigens, allofactors and allergens, thereby endowing said autoantigen, allofactor or allergen with the capacity to activate CD4+NKT cells. Direct elimination by killing of cells presenting said autoantigen, allofactor or allergen eliminates the capacity to mount an immune response against these antigens/factors.

The term “CD1d molecule” refers to a non-MHC derived molecule made of 3 alpha chains and an anti -parallel set of beta chains arranged into a deep hydrophobic groove opened on both sides and capable of presenting lipids, glycolipids or hydrophobic peptides to NKT cells. The term “immune disorders” or “immune diseases” refers to diseases wherein a reaction of the immune system is responsible for or sustains a malfunction or non- physiological situation in an organism.

The term “homologue” as used herein with reference to the epitopes used in the context of the invention, refers to molecules having at least 50%, at least 70%, at least 80%, at least 90%, at least 95% or at least 98% amino acid sequence identity with the naturally occurring epitope, thereby maintaining the ability of the epitope to bind an antibody or cell surface receptor of a B and/or T cell. Particular homologues of an epitope correspond to the natural epitope modified in at most three, more particularly in at most 2, most particularly in one amino acid.

The term “derivative” as used herein with reference to the peptides of the invention refers to molecules which contain at least the peptide active portion (i.e. the redox motif and the MHC class Il epitope capable of eliciting cytolytic CD4+ T cell activity) and, in addition thereto comprises a complementary portion which can have different purposes such as stabilising the peptides or altering the pharmacokinetic or pharmacodynamic properties of the peptide.

The term “sequence identity” of two sequences as used herein relates to the number of positions with identical nucleotides or amino acids divided by the number of nucleotides or amino acids in the shorter of the sequences, when the two sequences are aligned. In particular, the sequence identity is from 70% to 80%, from 81% to 85%, from 86% to 90%, from 91% to 95%, from 96% to 100%, or 100% calculated over the entire length of the sequence in question.

The terms “peptide-encoding polynucleotide (or nucleic acid)” and “polynucleotide (or nucleic acid) encoding peptide” as used herein refer to a nucleotide sequence, which, when expressed in an appropriate environment, results in the generation of the relevant peptide sequence or a derivative or homologue thereof. Such polynucleotides or nucleic acids include the normal sequences encoding the peptide, as well as derivatives and fragments of these nucleic acids capable of expressing a peptide with the required activity. The nucleic acid encoding a peptide according to the invention or fragment thereof is a sequence encoding the peptide or fragment thereof originating from a mammal or corresponding to a mammalian, most particularly a human peptide fragment. Such polynucleotides or nucleic acids molecules may be readily prepared using an automated synthesiser and the well-known codon-amino acid relationship of the genetic code. Such polynucleotides or nucleic acids may be incorporated into expression vectors, including plasmids, which are adapted for the expression of the polynucleotide or nucleic acid and production of the polypeptide in a suitable host such as bacterium, e.g. Escherichia coli, yeast cell, human cell, animal cell or plant cell. For therapeutic means, polynucleotides encoding the immunogenic peptides disclosed herein can be part of an expression system, cassette, plasmid or vector system such as viral and non-viral expression systems. Viral vectors known for therapeutic purposes are adenoviruses, adeno-associated viruses (AAVs), lentiviruses, and retroviruses. Non-viral vectors can be used as well and non-limiting examples include: transposon-based vector systems such as those derived from Sleeping Beauty (SB) or PiggyBac (PB). Nucleic acids can also be delivered through other carriers such as but not limited to nanoparticles, cationic lipids, liposomes etc. In a preferred embodiment, the nucleic acid encoding peptide is a non-immunogenic mRNA comprising N(1)-methyl-pseudouridine (m1Ψ). Design and synthesis of non-immunogenic mRNA is well-known in the art, such as in WO2018188730.

The term “immune disorders” or “immune diseases” refers to diseases wherein a reaction of the immune system is responsible for or sustains a malfunction or non-physiological situation in an organism. Included in immune disorders are, inter alia, allergic disorders and autoimmune diseases.

The terms “autoimmune disease” or “autoimmune disorder” refer to diseases that result from an aberrant immune response of an organism against its own cells and tissues due to a failure of the organism to recognise its own constituent parts (down to the sub-molecular level) as “self”. The group of diseases can be divided in two categories, organ-specific and systemic diseases. An “allergen” is defined as a substance, usually a macromolecule or a proteic composition which elicits the production of IgE antibodies in predisposed, particularly genetically disposed, individuals (atopics) patients. Similar definitions are presented in Liebers et al. (1996) Clin. Exp. Allergy 26, 494-516.

The term “demyelination” as used herein refers to damaging and/or degradation of myelin sheaths that surround axons of neurons which has as a consequence the formation of lesions or plaques. It is understood that the myelin acts as a protective covering surrounding nerve fibers in brain, optic nerves, and spinal cord. Due to demyelination, the signal conduction along the affected nerves is impaired (i.e. slowed or stopped), and may cause neurological symptoms such as deficiencies in sensation, movement, cognition, and/or other neurological function. The concrete symptoms a patient suffering from a demyelinating disease will vary depending on the disease and disease progression state. These may include a blurred and/or double vision, ataxia, clonus, dysarthria, fatigue, clumsiness, hand paralysis, hemiparesis, genital anaesthesia, incoordination, paraesthesia, ocular paralysis, impaired muscle coordination, muscle weakness, loss of sensation, impaired vision, neurological symptoms, unsteady way of walking (gait), spastic paraparesis, incontinence, hearing problems, speech problems, and others.

Therefore, “demyelinating diseases” or “demyelinating disorders” as used herein and commonly used in the art is indicative for any pathologic condition of the nervous system which involves impairment, for example damaging, or the myelin sheath of neurons. Demyelinating diseases may be stratified into central nervous system demyelinating diseases and peripheral nervous system. Alternatively, demyelinating diseases may be classified according to the cause of demyelination: destruction of myelin (demyelinating myelinoclastic), or abnormal and degenerative myelin (dysmyelinating leukodystrophic). Non-limiting examples of demyelinating diseases are Multiple Sclerosis (MS)-(e.g. Relapsing/Remitting Multiple Sclerosis, Secondary Progressive Multiple Sclerosis, Progressive Relapsing Multiple Sclerosis, Primary Progressive Multiple Sclerosis, and Acute Fulminant Multiple Sclerosis), Neuromyelitis Optica (NMO), Optic Neuritis, Acute Disseminated Encephalomyelitis, Balo's Disease, HTLV-I Associated Myelopathy, Schilder's Disease, Transverse Myelitis, Idiopathic inflammatory demyelinating diseases, vitamin B12-induced central nervous system neuropathies, Central pontine myelinolysis, Myelopathies including tabes dorsalis, Leukodystrophies such as Adrenoleukodystrophy, Leukoencephalopathies such as Progressive multifocal leukoencephalopathy (PML), and Rubella induced mental retardation. It is appreciated by a skilled person that several of the above mentioned annotations are general classification names indicative of a group of diseases characterized be an identical or similar set of aberrant processes at the molecular level and/or an identical or similar set of (clinical) symptoms. A human patient having a demyelinating disorder can have one or more symptoms of a demyelinating disorder such as, but not limited to, impaired vision, numbness, weakness in extremities, tremors or spasticity, heat intolerance, speech impairment, incontinence, dizziness, or impaired proprioception (e.g., balance, coordination, sense of limb position). A human (e.g., a human patient) with a family history of a demyelinating disorder (e.g., a genetic predisposition for a demyelinating disorder), or who exhibits mild or infrequent symptoms of a demyelinating disorder described above can be, for the purposes of the method, considered at risk of developing a demyelinating disorder (e.g., Multiple Sclerosis). Preferred demyelinating diseases in the context of the current disclosure are those caused by AQP4 autoantigens or involving anti-AQP4 antibodies, defined as “anti-AQP4 disease”, including but not limited to Neuromyelitis Optica (NMO) or NMOSD.

Anti-AQP4 autoantibodies have been identified in patients with multiple disorders also called “NMO Spectrum Disorders” (NMOSD or NMSD), “anti-AQP4 disease”, or “autoimmune aquaporin-4 channelopathy”, which can be used interchangeably herein. The spectrum of such diseases comprises in essence all diseases or disorders linked to the presence of anti-AQP4 auto-antibodies in the subject. Examples are: Devic's disease, such as Devic's disease encompassing single or recurrent events of longitudinally extensive (transverse) myelitis, and bilateral simultaneous or recurrent optic neuritis; Optic-Spinal MS (OSMS), previously considered a subtype of MS, encompassing brain lesions like in MS, but being a AQP4-positive; Longitudinally extensive (transverse) myelitis; Optic Neuritis associated with systemic autoimmune disease and with higher AQP4 autoantibody levels; Optic Neuritis or myelitis associated with lesions in specific brain areas such as the hypothalamus, periventricular nucleus, and brainstem; Tumefactive demyelination encompassing Tumefactive lesions. Devic's disease is currently considered a syndrome more than a disease, presenting an overlapping with the wide spectrum of multiple sclerosis in the form of Optic-Spinal MS.

An “antibody having B cell depleting activity” or “B cell depleting antibody” herein is an antibody or fragment that binds to a B cell marker which upon administration, results in demonstrable B cell depletion (i. e. reduction or suppression of circulating B cell levels). Such depletion may be achieved via various mechanisms such antibody-dependent cell-mediated cytotoxicity (ADCC) and/or complement dependent cytotoxicity (CDC), inhibition of B cell proliferation and/or induction of B cell death (e.g. via apoptosis). Preferably, such antibody, after administration, typically within about several days or less, will result in a depletion of B cell number by about 50% or more. In a preferred embodiment, the B cell depleting antibody will be Rituximab or ublituximab (chimeric anti CD20 antibodies) or one having substantially the same or greater cell depleting activity. In another preferred embodiment, the B cell depleting antibody will be Inebilizumab (chimeric anti CD19 antibody) or one having substantially the same or greater cell depleting activity.

A “B cell surface marker” herein is an antigen expressed on the surface of a B cell which can be targeted with an antagonist which binds thereto. Exemplary B cell surface markers include the CD10, CD19, CD20, CD21, CD22, CD23, CD24, CD37, CD53, CD72, CD73, CD74, CDw75, CDw76, CD77, CDw78, CD79a, CD79b, CD80 (B7.1), CD81, CD82, CD83, CDw84, CD85 and CD86 (B7.2) leukocyte surface markers. The B cell surface marker of particular interest is preferentially expressed on

B cells compared to other non-B cell tissues of a mammal and may be expressed on both precursor B cells and mature B cells. In one embodiment, the marker is one, like CD20 or CD 19, which is found on B cells throughout differentiation of the lineage from the stem cell stage up to a point just prior to terminal differentiation into plasma cells. The preferred B cell surface markers herein are CD19 or CD20.

The “CD20” antigen is a-35 kDa, non-glycosylated phosphoprotein found on the surface of greater than 90% of B cells from peripheral blood or lymphoid organs. CD20 is expressed during early pre-B cell development and remains until plasma cell differentiation. CD20 is present on both normal B cells as well as malignant B cells.

Other names for CD20 in the literature include “B-lymphocyte-restricted antigen” and“Bp35”. The CD20 antigen is described in Clark et al. PNAS (USA) 82: 1766 (1985).

The “CD19” antigen refers to a-90kDa antigen identified, for example, by the HD237-CD19 or B4 antibody (Kiesel et al. Leukemia Research II, 12: 1119 (1987)).

Like CD20, CD19 is found on cells throughout differentiation of the lineage from the stem cell stage up to a point just prior to terminal differentiation into plasma cells. Binding of an antagonist to CD19 may cause internalization of the CD19 antigen.

### Treatment with the B Cell Depleting Antibody

One or more compositions comprising a B cell depleting antibody will be formulated, dosed, and administered in a fashion consistent with good medical practice. As noted previously, the B cell depleting antibody and the immunogenic peptide, polynucleotides or cells according to the invention may be in the same or in different formulations. These formulations can be administered separately or concurrently, and in either order. Preferably, the B cell depleting antibody specific to the B cell antigen target, e.g.,

CD20 or CD19, will be administered separately from the immunogenic peptide polynucleotides or cells according to the invention. Even more preferably, the B cell depleting antibody will be administered before the immunogenic peptide polynucleotides or cells according to the invention. As an example only, a typical administration schedule of the B cell depleting antibody is one dose every 6 months.

As a general proposition, the therapeutically effective amount of an antibody administered parenterally per dose will typically be in the range of about 0.1 to 500 mg/kg of patient body weight per day, with the typical initial range of antagonist used being in the range of about 2 to 100 mg/kg.

The preferred B cell depleting antibody is Rituximab. Suitable dosage for such antibody is, for example, in the range from about 20 mg/m2 to about 1000 mg/m2.

The dosage of the antibody may be the same or different from that presently recommended for Rituximab for the treatment of non-Hodgkin's lymphoma. For example, one may administer to the patient one or more doses of substantially less than 375 mg/m2 of the antibody, e.g. where the dose is in the range from about 20 mg/m2 to about 250 mg2, for example from about 50 mg/m2 to about 200 mg/m2. Moreover, one may administer one or more initial doses of the antibody followed by one or more subsequent dose(s), wherein the mg/m2 dose of the antibody in the subsequent doses exceeds the mg/m2 dose of the antibody in the initial dose(s). For example, the initial dose may be in the range from about 20 mg/m2 to about 250 mg/m2 (e. g. from about 50 mg/m2 to about 200 mg/m2) and the subsequent dose may be in the range from about 250 mg/m2 to about 1000 mg/m2.

As noted above, however, these suggested amounts of antibodies are subject to a great deal of therapeutic discretion. The key factor in selecting an appropriate dose and scheduling is the result obtained.

The antibodies are administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antibody may suitably be administered by pulse infusion, e. g., with declining doses of the antibody. Preferably the dosing is given by injections, most preferably intravenous or subcutaneous injections, depending in part on whether the administration is brief or chronic.

The term “Neuromyelitis Optica” or “NMO”, also known as “Devic's disease”, refers to an autoimmune disorder in which white blood cells and antibodies primarily attack the optic nerves and the spinal cord, but may also attack the brain (reviewed in Wingerchuk 2006, Int MS J. 2006 May;13(2):42-50). The damage to the optic nerves produces swelling and inflammation that cause pain and loss of vision; the damage to the spinal cord causes weakness or paralysis in the legs or arms, loss of sensation, and problems with bladder and bowel function. NMO is a relapsing-remitting disease. During a relapse, new damage to the optic nerves and/or spinal cord can lead to accumulating disability. Unlike MS, there is no progressive phase of this disease. Therefore, preventing attacks is critical to a good long-term outcome. In cases associated with anti-AQP4 antibodies, it is considered that anti-AQP4 antibodies may trigger an attack on the myelin sheath resulting in demyelination. The cause of NMO in the majority of cases is due to a specific attack on auto-antigens. Up to a third of subjects may be positive for auto-antibodies directed against a component of myelin called Aquaporin-4 (AQP4). People with anti-AQP4 related NMO similarly can have episodes of transverse myelitis and optic neuritis.

The term “therapeutically effective amount” refers to an amount of the peptide of the invention or derivative thereof, which produces the desired therapeutic or preventive effect in a patient. For example, in reference to a disease or disorder, it is the amount which reduces to some extent one or more symptoms of the disease or disorder, and more particularly returns to normal, either partially or completely, the physiological or biochemical parameters associated with or causative of the disease or disorder. Typically, the therapeutically effective amount is the amount of the peptide of the invention or derivative thereof, which will lead to an improvement or restoration of the normal physiological situation. For instance, when used to therapeutically treat a mammal affected by an immune disorder, it is a daily amount peptide/kg body weight of the said mammal. Alternatively, where the administration is through gene-therapy, the amount of naked DNA or viral vectors is adjusted to ensure the local production of the relevant dosage of the peptide of the invention, derivative or homologue thereof. The term “natural” when referring to a peptide relates to the fact that the sequence is identical to a fragment of a naturally occurring protein (wild type or mutant). In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence.

Further envisaged is the combined treatment with the immunogenic peptide as defined herein in combination with further active ingredients or current and future treatment methods or strategies for alleviating the symptoms of the disease or attacks and relapses thereof, or for preventing attacks and relapses. These include but are not limited to: administering corticosteroids, immunosuppressants, or fumarates.

The term “oxidoreductase motif”, “thiol-oxidoreductase motif”, “thioreductase motif”, “thioredox motif” or “redox motif” are used here as synonymous terms and refers to a motif of general sequence thioreductase sequence motif C-Xₙ-[CST]- or [CST]-Xₙ-C-, with n being an integer from 0 to 6. Such peptide motives exert reducing activity for disulfide bonds on proteins (such as enzymes) through redox active cysteines within conserved active domain consensus sequences: C-Xₙ-[CST]- or [CST]-Xₙ-C-, such as for example in CXXC (SEQ ID NO: 222), C-XX-S (SEQ ID NO: 223), C-XX-T (SEQ ID NO: 224), S-XX-C (SEQ ID NO: 225), T-XX-C (SEQ ID NO: 226) (Fomenko et al. (2003) Biochemistry 42, 1 1214-1 1225), in which “X” stands for any amino acid, in which C stands for cysteine, S for serine, T for threonine and X for any amino acid except tyrosine, phenylalanine or tryptophan.

The terms “cysteine”, “C”, “serine”, “S”, and “threonine”, “T”, when used in the light of the amino acid residues present in the oxidoreductase motifs disclosed herein respectively refer to naturally occurring cysteine, serine or threonine amino acids. Unless explicitly stated differently, said terms hence exclude chemically modified cysteines, serines and threonines such as those modified so as to carry an acetyl, methyl, ethyl or propionyl group, either on the N-terminal amide of the amino acid residue of the motif or on the C-terminal carboxy group.

In a further embodiment thereto, said oxidoreductase motif is positioned N-terminally of the T-cell epitope.

Alternatively, the immunogenic peptides may contain an oxidoreductase motif in the form of the following general amino acid formula: Zₘ-[CST]-Xₙ-C- or Zₘ-C-Xₙ-[CST]-, wherein n is an integer chosen from 0 to 6, wherein m is an integer selected from 0 to 3, wherein X is any amino acid, wherein Z is any amino acid, in which C stands for cysteine, S for serine, T for threonine.

Preferably said oxidoreductase motif is not part of a repeat of the standard C-XX-[CST] or [CST]-XX-C oxidoreductase motifs such as repeats of said motif which can be spaced from each other by one or more amino acids (e.g. CXXC X CXXC X CXXC (SEQ ID NO: 227)), as repeats which are adjacent to each other (CXXCCXXCCXXC (SEQ ID NO: 228)) or as repeats which overlap with each other CXXCXXCXXC (SEQ ID NO: 229) or CXCCXCCXCC (SEQ ID NO: 230)), especially when n is 0 or 1 and m is 0.

Hence, envisaged are thus motifs of the form Zₘ-[CST]-C- or Zₘ-C-[CST]-, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2 and Z is a basic amino acid selected from: H, K, R, and a non-natural basic amino acid as defined herein, such as L-ornithine, preferably K or H. Non-limiting preferred examples of such motifs are KCC, KKCC (SEQ ID NO: 231), RCC, RRCC (SEQ ID NO: 232), RKCC (SEQ ID NO: 233), or KRCC (SEQ ID NO: 234).

Further envisaged are motifs of the form Zₘ-[CST]-X-C- or Zₘ-C-X-[CST]-, wherein X is any amino acid, preferably a basic amino acid selected from: H, K, R, and a non-natural basic amino acid such as L-ornithine, preferably K or R, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2 and Z is a basic amino acid selected from: H, K, R, and a non-natural basic amino acid as defined herein, such as L-ornithine, preferably K or H. Non-limiting preferred examples of such motifs are KCXC (SEQ ID NO: 235), KKCXC (SEQ ID NO: 236), RCXC (SEQ ID NO:

237), RRCXC (SEQ ID NO: 238), RKCXC (SEQ ID NO: 239), KRCXC (SEQ ID NO: 240), KCKC (SEQ ID NO: 241), KKCKC (SEQ ID NO: 242), KCRC (SEQ ID NO: 243), KKCRC (SEQ ID NO: 244), RCRC (SEQ ID NO: 245), RRCRC (SEQ ID NO: 246), RKCKC (SEQ ID NO: 247), KRCKC (SEQ ID NO: 248), RCKC (SEQ ID NO: 249), HCRC (SEQ ID NO: 320) or KHCRC (SEQ ID NO: 321).

Further envisaged are motifs of the form Zₘ-[CST]-XX-C- or Zₘ-C-XX-[CST] -. In these motifs, an internal X¹X² amino acid couple is situated within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2 and Z is a basic amino acid selected from: H, K, or R, or a non-natural basic amino acid as defined herein, such as L-ornithine, preferably K or H. X¹ and X2, each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a further example, at least one of X¹ or X² in said motif is a basic amino acid selected from: H, K, or R, or a non-natural basic amino acid as defined herein, such as L-ornithine. In another example of the motif, at least one of X¹ or X² in said motif is P or Y. Specific non-limiting examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH. Particularly preferred motifs of this form are CPYC, HCPYC, KHCPYC, KCPYC, RCPYC, KKCPYC, KRCPYC, CHGC, HCGHC, KCGHC, KHCGHC, RCGHC, KKCGHC, and KRCGHC (SEQ ID NO: 157 to 170).

Further envisaged are motifs of the form Zₘ-[CST]-XXX-C- or Zₘ-C-XXX-[CST]-, thereby creating an internal X¹X²X³ amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2 and Z is a basic amino acid selected from: H, K, or R, or a non-natural basic amino acid as defined herein, such as L-ornithine, preferably K or H. In certain examples, X¹, X², and X³, each individually can be any amino acid selected from the group consisting of: G, A, V, L, I, M, F, W, P, S, T, C, Y, N, Q, D, E, K, R, and H, or non-natural amino acids. Preferably, X¹, X², and X³ in said motif is any amino acid except for C, S, or T. In a specific embodiment, at least one of X¹, X², or X³ in said motif is a basic amino acid selected from: H, K, or R, or a non-natural basic amino acid as defined herein, such as L-ornithine. Specific examples of the internal X¹X²X³ amino acid stretch within the oxidoreductase motif are: XPY, PXY, and PYX, wherein X can be can be any amino acid, preferably a basic amino acid such as K, R, or H, or a non-natural basic amino acid such as L-ornithine. Non-limiting examples include KPY, RPY, HPY, GPY, APY, VPY, LPY, IPY, MPY, FPY, WPY, PPY, SPY, TPY, CPY, YPY, NPY, QPY, DPY, EPY, KPY, PKY, PRY, PHY, PGY, PAY, PVY, PLY, PIY, PMY, PFY, PWY, PPY, PSY, PTY, PCY, PYY, PNY, PQY, PDY, PEY, PLY, PYK, PYR, PYH, PYG, PYA, PYV, PYL, PYI, PYM, PYF, PYW, PYP, PYS, PYT, PYC, PYY, PYN, PYQ, PYD, or PYE. Alternative examples of the internal X¹X²X³ amino acid stretch within the oxidoreductase motif are XHG, HXG, and HGX, wherein X can be any amino acid, such as in KHG, RHG, HHG, GHG, AHG, VHG, LHG, IHG, MHG, FHG, WHG, PHG, SHG, THG, CHG, YHG, NHG, QHG, DHG, EHG, and KHG, HKG, HRG, HHG, HGG, HAG, HVG, HLG, HIG, HMG, HFG, HWG, HPG, HSG, HTG, HCG, HYG, HNG, HQG, HDG, HEG, HLG, HGK, HGR, HGH, HGG, HGA, HGV, HGL, HGI, HGM, HGF, HGW, HGP, HGS, HGT, HGC, HGY, HGN, HGQ, HGD, or HGE. Yet alternative examples of the internal X¹X²X³ amino acid stretch within the oxidoreductase motif are XGP, GXP, and GPX, wherein X can be any amino acid, such as in KGP, RGP, HGP, GGP, AGP, VGP, LGP, IGP, MGP, FGP, WGP, PGP, SGP, TGP, CGP, YGP, NGP, QGP, DGP, EGP, KGP, GKP, GRP, GHP, GGP, GAP, GVP, GLP, GIP, GMP, GFP, GWP, GPP, GSP, GTP, GCP, GYP, GNP, GQP, GDP, GEP, GLP, GPK, GPR, GPH, GPG, GPA, GPV, GPL, GPI, GPM, GPF, GPW, GPP, GPS, GPT, GPC, GPY, GPN, GPQ, GPD, or GPE. Yet alternative examples of the internal X¹X²X³ amino acid stretch within the oxidoreductase motif are XGH, GXH, and GHX, wherein X can be any amino acid, such as in KGH, RGH, HGH, GGH, AGH, VGH, LGH, IGH, MGH, FGH, WGH, PGH, SGH, TGH, CGH, YGH, NGH, QGH, DGH, EGH, KGH, GKH, GRH, GHH, GGH, GAH, GVH, GLH, GIH, GMH, GFH, GWH, GPH, GSH, GTH, GCH, GYH, GNH, GQH, GDH, GEH, GLH, GHK, GHR, GHH, GHG, GHA, GHV, GHL, GHI, GHM, GHF, GHW, GHP, GHS, GHT, GHC, GHY, GHN, GHQ, GHD, or GHE. Yet alternative examples of the internal X¹X²X³ amino acid stretch within the oxidoreductase motif are XGF, GXF, and GFX, wherein X can be any amino acid, such as in KGF, RGF, HGF, GGF, AGF, VGF, LGF, IGF, MGF, FGF, WGF, PGF, SGF, TGF, CGF, YGF, NGF, QGF, DGF, EGF, and KGF, GKF, GRF, GHF, GGF, GAF, GVF, GLF, GIF, GMF, GFF, GWF, GPF, GSF, GTF, GCF, GYF, GNF, GQF, GDF, GEF, GLF, GFK, GFR, GFH, GFG, GFA, GFV, GFL, GFI, GFM, GFF, GFW, GFP, GFS, GFT, GFC, GFY, GFN, GFQ, GFD, or GFE. Yet alternative examples of the internal X¹X²X³ amino acid stretch within the oxidoreductase motif are XRL, RXL, and RLX, wherein X can be any amino acid, such as in KRL, RRL, HRL, GRL, ARL, VRL, LRL, IRL, MRL, FRL, WRL, PRL, SRL, TRL, CRL, YRL, NRL, QRLRL, DRL, ERL, KRL, GKF, GRF, GHF, GGF, GAF, GVF, GLF, GIF, GMF, GFF, GWF, GPF, GSF, GTF, GCF, GYF, GNF, GQF, GDF, GEF, and GLF, RLK, RLR, RLH, RLG, RLA, RLV, RLL, RLI, RLM, RLF, RLW, RLP, RLS, RLT, RLC, RLY, RLN, RLQ, RLD, or RLE. Yet alternative examples of the internal X¹X²X³ amino acid stretch within the oxidoreductase motif are XHP, HXP, and HPX, wherein X can be any amino acid, such as in KHP, RHP, HHP, GHP, AHP, VHP, LHP, IHP, MHP, FHP, WHP, PHP, SHP, THP, CHP, YHP, NHP, QHP, DHP, EHP, KHP, HKP, HRP, HHP, HGP, HAF, HVF, HLF, HIF, HMF, HFF, HWF, HPF, HSF, HTF, HCF, HYP, HNF, HOF, HDF, HEF, HLP, HPK, HPR, HPH, HPG, HPA, HPV, HPL, HPI, HPM, HPF, HPW, HPP, HPS, HPT, HPC, HPY, HPN, HPQ, HPD, or HPE.

Particularly preferred examples are: CRPYC (SEQ ID NO: 250), KCRPYC (SEQ ID NO: 251), KHCRPYC (SEQ ID NO: 252), RCRPYC (SEQ ID NO: 253), HCRPYC (SEQ ID NO: 254), CPRYC (SEQ ID NO: 255), KCPRYC (SEQ ID NO: 256), RCPRYC (SEQ ID NO: 257), HCPRYC (SEQ ID NO: 258), CPYRC (SEQ ID NO: 259), KCPYRC (SEQ ID NO: 260), RCPYRC (SEQ ID NO: 261), HCPYRC (SEQ ID NO: 262), CKPYC (SEQ ID NO: 263), KCKPYC (SEQ ID NO: 264), RCKPYC (SEQ ID NO: 265), HCKPYC (SEQ ID NO: 266), CPKYC (SEQ ID NO: 267), KCPKYC (SEQ ID NO: 268), RCPKYC (SEQ ID NO: 269), HCPKYC (SEQ ID NO: 270), CPYKC (SEQ ID NO: 271), KCPYKC (SEQ ID NO: 272), RCPYKC (SEQ ID NO: 273), and HCPYKC (SEQ ID NOs: 274).

Further envisaged are motifs of the form Zₘ-[CST]-XXXX-C- or Zₘ-C-XXXX-[CST]-, thereby creating an internal X¹X²X³X⁴ (SEQ ID NO: 275) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2 and Z is a basic amino acid selected from: H, K, or R, or a non-natural basic amino acid as defined herein, such as L-ornithine, preferably K or H. X¹, X², X³ and X⁴ each individually can be any amino acid selected from the group consisting of: G, A, V, L, I, M, F, W, P, S, T, C, Y, N, Q, D, E, K, R, and H, or non-natural amino acids as defined herein. Preferably, X¹, X², X³ and X⁴ in said motif is any amino acid except for C, S, or T. In certain non-limiting examples, at least one of X¹, X², X³ or X⁴ in said motif is a basic amino acid selected from: H, K, or R, or a non-natural basic amino acid as defined herein. Specific examples include LAVL (SEQ ID NO: 276), TVQA (SEQ ID NO: 277) or GAVH (SEQ ID NO: 278) and their variants such as: X¹AVL, LX²VL, LAX³L, or LAVX⁴; X¹VQA, TX²QA, TVX³A, or TVQX⁴; X¹AVH, GX²VH, GAX³H, or GAVX⁴ (corresponding to SEQ ID NO: 279 to 290); wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein.

Further envisaged are motifs of the form Zₘ-[CST]-XXXXX-C- or Zₘ-C-XXXXX-[CST]-, thereby creating an internal X¹X²X³X⁴X⁵ (SEQ ID NO: 291) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2 and Z is a basic amino acid selected from: H, K, or R, or a non-natural basic amino acid as defined herein, such as L-ornithine, preferably K or

H. X¹, X², X³, X⁴ and X⁵ each individually can be any amino acid selected from the group consisting of: G, A, V, L, I, M, F, W, P, S, T, C, Y, N, Q, D, E, K, R, and H, or non-natural amino acids. Preferably, X¹, X², X³, X⁴ and X⁵ in said motif is any amino acid except for C, S, or T. In certain examples, at least one of X¹, X², X³, X⁴, or X⁵ in said motif is a basic amino acid selected from: H, K, or R, or a non-natural basic amino acid as defined herein. Specific examples include PAFPL (SEQ ID NO: 292) or DQGGE (SEQ ID NO: 293) and their variants such as: X¹AFPL, PX²FPL, PAX³PL, PAFX⁴L, or PAFPX⁵; X¹QGGE, DX²GGE, DQX³GE, DQGX⁴E, or DQGGX⁵ (corresponding to SEQ ID NO: 294 to 303), wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural amino acids as defined herein.

Further envisaged are motifs of the form Zₘ-[CST]-XXXXXX-C- or Zₘ-C-XXXXXX-[CST]- as defined in aspect 1, wherein n is 6, thereby creating an internal X¹X²X³X⁴X⁵X⁶ (SEQ ID NO: 304) amino acid stretch within the oxidoreductase motif, wherein m is an integer selected from 0 to 3, wherein Z is any amino acid, preferably a basic amino acid selected from: H, K, R, and a non-natural basic amino acid as defined herein, such as L-ornithine, preferably K or H. Preferred are motifs wherein m is 1 or 2 and Z is a basic amino acid selected from: H, K, or R, or a non-natural basic amino acid as defined herein, such as L-ornithine, preferably K or H. X¹, X², X³, X⁴ X⁵ and X⁶ each individually can be any amino acid selected from the group consisting of: G, A, V, L, I, M, F, W, P, S, T, C, Y, N, Q, D, E, K, R, and H, or non-natural amino acid. Preferably, X¹, X², X³, X⁴, X⁵ and X⁶ in said motif is any amino acid except for C, S, or T. In certain examples, at least one of X¹, X², X³, X⁴, X⁵ or X⁶ in said motif is a basic amino acid selected from: H, K, or R, or a non-natural basic amino acid as defined herein. Specific examples include DIADKY (SEQ ID NO: 305) or variants thereof such as: X¹IADKY, DX²ADKY, DIX³DKY, DIAX⁴KY, DIADX⁵Y, or DIADKX⁶ (corresponding to SEQ ID NO: 306 to 311), wherein X¹, X², X³, X⁴, X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein. Further envisaged are motifs of the form Zₘ-[CST]-Xₙ-C- or Zₘ-C-Xₙ-[CST]-, wherein n is 0 to 6 and wherein m is 0 (i.e. [CST]-Xₙ-C- or C-Xₙ-[CST]-), and wherein one of the C or [CST] residues has been modified so as to carry an acetyl, methyl, ethyl or propionyl group, either on the N-terminal amide of the amino acid residue of the motif or on the C-terminal carboxy group. In preferred embodiments of such a motif, n is 2 and m is 0, wherein the internal X¹X², each individually, can be any amino acid selected from the group consisting of: G, A, V, L, I, M, F, W, P, S, T, C, Y, N, Q, D, E, K, R, and H, or non-natural amino acids. Preferably, X¹ and X² in said motif is any amino acid except for C, S, or T. In a further example, at least one of X¹ or X² in said motif is a basic amino acid selected from: H, K, or R, or a non-natural basic amino acid as defined herein, such as L-ornithine. In another example of the motif, at least one of X¹ or X² in said motif is P or Y. Specific non-limiting examples of the internal X¹X² amino acid couple within the oxidoreductase motif: PY, HY, KY, RY, PH, PK, PR, HG, KG, RG, HH, HK, HR, GP, HP, KP, RP, GH, GK, GR, GH, KH, and RH. Preferably said modification results in an N-terminal acetylation of the first cysteine in the motif (N-acetyl-cysteine).

The redox motif in the above immunogenic peptides is placed either immediately adjacent to the T cell epitope sequence within the immunogenic peptide, or is separated from the T cell epitope by a linker sequence (“linker”). More particularly, the linker comprises an amino acid sequence of 7 amino acids or less. Most particularly, the linker comprises 1, 2, 3, or 4, 5, 6, or 7 amino acids. Said linker can be encompassing amino acids that are flanking the epitope in the natural AQP4 amino acid sequence or can be different from these amino acids.

In addition, the immunogenic peptides can have a flanking sequence (“flanker”) following the epitope sequence (at its C-terminus). More particularly, the flanker comprises an amino acid sequence of 7 amino acids or less. Most particularly, the linker comprises 1, 2, 3, or 4, 5, 6, or 7 amino acids. Said flanker can be encompassing amino acids that are flanking the epitope in the natural AQP4 amino acid sequence or can be different from these amino acids.

The sequence of the linker and/or flanking sequence can have an influence on the immunogenicity of the immunogenic peptide as a whole.

The term Aquaporin-4 or AQP4 refers to the human protein encoded by the aquaporin gene. The terms AQP4 (protein) or Aquarporin-4 as used herein are defined by the amino acid sequence corresponding to NCBI Gene 361, and UniProtKB identifier P55087 (AQP4_HUMAN) (SEQ ID NO: 136):

MSDRPTARRWGKCGPLCTRENIMVAFKGVWTQAFWKAVTAEFLAMLIFV
LLSLGSTINWGGTEKPLPVDMVLISLCFGLSIATMVQCFGHISGGHINP
AVTVAMVCTRKISIAKSVFYIAAQCLGAIIGAGILYLVTPPSVVGGLGV
TMVHGNLTAGHGLLVELIITFQLVFTIFASCDSKRTDVTGSIALAIGFS
VAIGHLFAINYTGASMNPARSFGPAVIMGNWENHWIYWVGPIIGAVLAG
GLYEYVFCPDVEFKRRFKEAFSKAAQQTKGSYMEVEDNRSQVETDDLIL
KPGVVHVIDVDRGEEKKGKDQSGEVLSSV

Aquaporin-4 (AQP4) is one of the most abundant molecules in the brain and is particularly prevalent in astrocytic membranes at the blood-brain and brain-liquor interfaces. While AQP4 has been implicated in a number of pathophysiological processes, its role in brain physiology has remained elusive. Only recently has evidence accumulated to suggest that AQP4 is involved in such diverse functions as regulation of extracellular space volume, potassium buffering, cerebrospinal fluid circulation, interstitial fluid resorption, waste clearance, neuroinflammation, osmosensation, cell migration, and Ca2+ signaling. AQP4 is also required for normal function of the retina, inner ear, and olfactory system. A review will be provided of the physiological roles of AQP4 in brain and of the growing list of data that emphasize the polarized nature of astrocytes (Nagelhus and Ottersen, Physiol Rev. 2013 Oct; 93(4): 1543-1562).

Amino acids are referred to herein with their full name, their three-letter abbreviation or their one letter abbreviation.

Motifs of amino acid sequences are written herein according to the format of Prosite. Motifs are used to describe a certain sequence variety at specific parts of a sequence. The symbol X is used for a position where any amino acid is accepted. Alternatives are indicated by listing the acceptable amino acids for a given position, between square brackets ('[]'). For example: [CST] stands for an amino acid selected from Cys, Ser or Thr. Amino acids which are excluded as alternatives are indicated by listing them between curly brackets ('{ }'). For example: {AM} stands for any amino acid except Ala and Met. The different elements in a motif are optionally separated from each other by a hyphen (-). In the context of the motifs disclosed in this specification, the disclosed general oxidoreductase motifs are typically accompanied by a hyphen not forming a connection with a different element outside the motif. These ‘open’ hyphens indicate the position of the physical connection of the motif with another portion of the immunogenic peptide such as a linker sequence or an epitope sequence. For example, a motif of the form “Zₘ-C-Xₙ-[CST]-” indicates that the [CST] is the amino acid connected to the other portion of the immunogenic peptide, and Z is a terminal amino acid of the immunogenic peptide. Preferred physical connections are peptide bonds.

Repetition of an identical element within a motif can be indicated by placing behind that element a numerical value or a numerical range between parentheses. For example In this respect, “Xₙ” refers to n-times “X”. X(2) corresponds to X-X or XX; X(2, 5) corresponds to 2, 3, 4 or 5 X amino acids, A(3) corresponds to A-A-A or AAA. To distinguish between the amino acids, those outside the oxidoreductase motif can be called external amino acids, those within the redox motif are called internal amino acids. Unless stated otherwise X represents any amino acid, particularly an L-amino acid, more particularly one of the 20 naturally occurring L-amino acids.

Any one of the peptides disclosed herein, comprising a T cell epitope of AQP4 and a modified peptide motif sequence, having reducing activity is capable of generating a population of antigen-specific cytolytic CD4+ T cells or NKT cells towards antigen-presenting cells. T cell epitopes can be MHC class Il epitopes typically having a length of 9 amino acids or NKT cell epitopes typically having a length of 7 amino acids.

Accordingly, in its broadest sense, the invention relates to peptides which comprise at least one T-cell epitope of AQP4 with a potential to trigger an immune reaction, and a modified oxidoreductase sequence motif with a reducing activity on peptide disulfide bonds. The T cell epitope and the modified oxidoreductase motif sequence may be immediately adjacent to each other in the peptide or optionally separated by one or more amino acids (a so called linker sequence).

Optionally the peptide additionally comprises an endosome targeting sequence and/or additional “flanking” sequences.

The peptides of the invention comprise an MHC class II T-cell epitope or NKT cell epitope of AQP4 with a potential to trigger an immune reaction, and a modified oxidoreductase motif. The reducing activity of the motif sequence in the peptide can be assayed for its ability to reduce a sulfhydryl group such as in the insulin solubility assay wherein the solubility of insulin is altered upon reduction, or with a fluorescence-labelled substrate such as insulin. An example of such assay uses a fluorescent peptide and is described in Tomazzolli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again.

The modified oxidoreductase motif may be positioned at the amino-terminus side of the T-cell epitope or at the carboxy-terminus of the T-cell epitope.

As explained in detail further on, the peptides of the present invention can be made by chemical synthesis, which allows the incorporation of non-natural amino acids. Accordingly, “C” in the above recited oxidoreductase motifs represents either cysteine or another amino acid with a thiol group such as mercaptovaline, homocysteine or other natural or non-natural amino acids with a thiol function. In order to have reducing activity, the cysteines present in a modified oxidoreductase motif should not occur as part of a cystine disulfide bridge. X can be any of the 20 natural amino acids, including S, C, or T or can be a non-natural amino acid. In particular embodiments X is an amino acid with a small side chain such as Gly, Ala, Ser or Thr. In further particular embodiments, X is not an amino acid with a bulky side chain such as Trp. In further particular embodiments X is not Cysteine. In further particular embodiments at least one X in the modified oxidoreductase motif is His. In other further particular embodiments at least one X in the modified oxidoreductase is Pro.

Peptides may further comprise modifications to increase stability or solubility, such as modification of the N-terminal NH₂ group or the C terminal COOH group (e.g. modification of the COOH into a CONH₂ group).

In the peptides of the present invention comprising a modified oxidoreductase motif, the motif is located such that, when the epitope fits into the MHC or CD1d groove, the motif remains outside of the MHC or CD1d binding groove. The modified oxidoreductase motif is placed either immediately adjacent to the epitope sequence within the peptide [in other words a linker sequence of zero amino acids between motif and epitope], or is separated from the T cell epitope by a linker comprising an amino acid sequence of 7 amino acids or less. More particularly, the linker comprises 1, 2, 3, 4, 5, 6, or 7 amino acids. Specific embodiments are peptides with a 0, 1, 2 or 3 amino acid linker between epitope sequence and modified oxidoreductase motif sequence. In those peptides where the modified oxidoreductase motif sequence is adjacent to the epitope sequence this is indicated as position P-4 to P-1 or P+1 to P+4 compared to the epitope sequence. Apart from a peptide linker, other organic compounds can be used as linker to link the parts of the peptide to each other (e.g. the modified oxidoreductase motif sequence to the T cell epitope sequence).

The peptides of the present invention can further comprise additional short amino acid sequences N or C-terminally of the sequence comprising the T cell epitope and the modified oxidoreductase motif. Such an amino acid sequence is generally referred to herein as a “flanking sequence”. A flanking sequence can be positioned between the epitope and an endosomal targeting sequence and/or between the modified oxidoreductase motif and an endosomal targeting sequence. In certain peptides, not comprising an endosomal targeting sequence, a short amino acid sequence may be present N and/or C terminally of the modified oxidoreductase motif and/or epitope sequence in the peptide. More particularly a flanking sequence is a sequence of between 1 and 7 amino acids, most particularly a sequence of 1, 2, or 3 amino acids.

Preferably Z in the oxidoreductase motif corresponds to the N- or C-terminal end of the immunogenic peptide.

The modified oxidoreductase motif may be located N-terminal from the epitope.

In certain embodiments of the present invention, peptides are provided comprising one epitope sequence and a modified oxidoreductase motif sequence. In further particular embodiments, the modified oxidoreductase motif occurs several times (1, 2, 3, 4 or even more times) in the peptide, for example as repeats of the modified oxidoreductase motif which can be spaced from each other by one or more amino acids or as repeats which are immediately adjacent to each other. Alternatively, one or more modified oxidoreductase motifs are provided at both the N and the C terminus of the T cell epitope sequence.

Other variations envisaged for the peptides of the present invention include peptides which contain repeats of a T cell epitope sequence wherein each epitope sequence is preceded and/or followed by the modified oxidoreductase motif (e.g. repeats of “modified oxidoreductase motif-epitope” or repeats of “modified oxidoreductase motif-epitope-modified oxidoreductase motif’). Herein the modified oxidoreductase motifs can all have the same sequence but this is not obligatory. It is noted that repetitive sequences of peptides which comprise an epitope which in itself comprises the modified oxidoreductase motif will also result in a sequence comprising both the ‘epitope’ and a ‘modified oxidoreductase motif’. In such peptides, the modified oxidoreductase motif within one epitope sequence functions as a modified oxidoreductase motif outside a second epitope sequence.

Typically the peptides of the present invention comprise only one T cell epitope. As described below a T cell epitope in a protein sequence can be identified by functional assays and/or one or more in silica prediction assays. The amino acids in a T cell epitope sequence are numbered according to their position in the binding groove of the MHC proteins. A T-cell epitope present within a peptide consist of between 8 and 25 amino acids, yet more particularly of between 8 and 16 amino acids, yet most particularly consists of 8, 9, 10, 11, 12, 13, 14, 15 or 16 amino acids.

In a more particular embodiment, the T cell epitope consists of a sequence of 9 amino acids. In a further particular embodiment, the T-cell epitope is an epitope, which is presented to T cells by MHC-class II molecules [MHC class Il restricted T cell epitopes]. In an alternative embodiment, the T-cell epitope is an NKT cell epitope, which is presented to T cells by CD1d molecules [NKT cell specific epitopes]. Typically T cell epitope sequence refers to the octapeptide or more specifically nonapeptide sequence which fits into the cleft of an MHC II protein or CD1d protein.

The T cell epitope of the peptides of the present invention can correspond either to a natural epitope sequence of a protein or can be a modified version thereof, provided the modified T cell epitope retains its ability to bind within the MHC or CD1d cleft, similar to the natural T cell epitope sequence. The modified T cell epitope can have the same binding affinity for the MHC or CD1d protein as the natural epitope, but can also have a lowered affinity. In particular, the binding affinity of the modified peptide is no less than 10-fold less than the original peptide, more particularly no less than 5 times less. Peptides of the present invention have a stabilising effect on protein complexes. Accordingly, the stabilising effect of the peptide-MHC/CD1d complex compensates for the lowered affinity of the modified epitope for the MHC or CD1d molecule.

The sequence comprising the T cell epitope and the reducing compound within the peptide can be further linked to an amino acid sequence (or another organic compound) that facilitates uptake of the peptide into late endosomes for processing and presentation within MHC class Il or CD1d determinants. The late endosome targeting is mediated by signals present in the cytoplasmic tail of proteins and correspond to well-identified peptide motifs. The late endosome targeting is mediated by signals present in the cytoplasmic tail of proteins and correspond to well-identified peptide motifs such as the dileucine-based [DE]XXXL[LI] (SEQ ID NO: 312) or DXXLL motif (SEQ ID NO: 313) (e.g. DXXXLL, SEQ ID NO 314)), the tyrosine-based YXX0 motif or the so-called acidic cluster motif (SEQ ID NO: 315). The symbol 0 represents amino acid residues with a bulky hydrophobic side chains such as Phe, Tyr and Trp.

The late endosome targeting sequences allow for processing and efficient presentation of the antigen-derived T cell epitope by MHC class Il or CD1d molecules. Such endosomal targeting sequences are contained, for example, within the gp75 protein (Vijayasaradhi et al. (1995) J. Cell. Biol. 130, 807-820), the human CD3 gamma protein, the HLA-BM 11 (Copier et al. (1996) J. Immunol. 157, 1017-1027), the cytoplasmic tail of the DEC205 receptor (Mahnke et al. (2000) J. Cell Biol. 151, 673-683). Other examples of peptides which function as sorting signals to the endosome are disclosed in the review of Bonifacio and Traub (2003) Annu. Rev. Biochem. 72, 395-447. Alternatively, the sequence can be that of a subdominant or minor T cell epitope from a protein, which facilitates uptake in late endosome without overcoming the T cell response towards the antigen. The late endosome targeting sequence can be located either at the amino-terminal or at the carboxy-terminal end of the antigen derived peptide for efficient uptake and processing and can also be coupled through a flanking sequence, such as a peptide sequence of up to 10 amino acids. When using a minor T cell epitope for targeting purpose, the latter is typically located at the amino-terminal end of the antigen derived peptide.

Accordingly, the present invention envisages peptides of antigenic proteins and their use in eliciting specific immune reactions. These peptides can either correspond to fragments of proteins which comprise, within their sequence i.e. a reducing compound and a T cell epitope separated by at most 10, preferably 7 amino acids or less. Alternatively, and for most antigenic proteins, the peptides of the invention are generated by coupling a reducing compound, more particularly a reducing modified oxidoreductase motif as described herein, N-terminally or C-terminally to a T cell epitope of the antigenic protein (either directly adjacent thereto or with a linker of at most 10, more particularly at most 7 amino acids). Moreover the T cell epitope sequence of the protein and/or the modified oxidoreductase motif can be modified and/or one or more flanking sequences and/or a targeting sequence can be introduced (or modified), compared to the naturally occurring sequence. Thus, depending on whether or not the features of the present invention can be found within the sequence of the antigenic protein of interest, the peptides of the present invention can comprise a sequence which is ‘artificial’ or ‘naturally occurring’.

The term “natural” when referring to a peptide relates to the fact that the sequence is identical to a fragment of a naturally occurring protein (wild type or mutant). In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence.

The peptides of the present invention can vary substantially in length. The length of the peptides can vary from 9 or 11 amino acids, i.e. consisting of an epitope of 7-9 amino acids, adjacent thereto the modified oxidoreductase motif of from 2 to 11 amino acids, up to 12, 13, 14, 15, 16, 17, 18, 19, 20, 25, 30, 40 or 50 amino acids. For example, a peptide may comprise an endosomal targeting sequence of 40 amino acids, a flanking sequence of about 2 amino acids, an oxidoreductase motif as described herein of 2 to 11 amino acids, a linker of 4 to 7 amino acids and a T cell epitope peptide of 7, 8 or 9 amino acids minimal length.

Accordingly, in particular embodiments, the complete peptide consists of between 9 amino acids up 20, 25, 30, 40, 50, 75 or 100 amino acids. More particularly, where the reducing compound is a modified oxidoreductase motif as described herein, the length of the (artificial or natural) sequence comprising the epitope and modified oxidoreductase motif optionally connected by a linker (referred to herein as ‘epitope-modified oxidoreductase motif’ sequence), without the endosomal targeting sequence, is critical. The ‘epitope-modified oxidoreductase motif’ more particularly has a length of 9, 10, 11, 12, 13, 14, 15, 16, 17, 18 or 19 amino acids. Such peptides of 9, 10, 11, 12, 13 or 14 to 19 amino acids can optionally be coupled to an endosomal targeting signal of which the size is less critical.

As detailed above, in particular embodiments, the peptides of the present invention comprise a reducing modified oxidoreductase motif as described herein linked to a T cell epitope sequence.

In further particular embodiments, the peptides of the invention are peptides comprising T cell epitopes which do not comprise an amino acid sequence with oxidoreductase properties within their natural sequence.

Generally, the peptides of the present invention are not natural (thus no fragments of proteins as such) but artificial peptides which contain, in addition to a T cell epitope, a modified oxidoreductase motif as described herein, whereby the modified oxidoreductase motif is immediately separated from the T cell epitope by a linker consisting of up to seven, most particularly up to four or up to 2 amino acids.

It has been shown that upon administration (i.e. injection) to a mammal of a peptide according to the invention (or a composition comprising such a peptide), the peptide elicits the activation of T cells recognising the antigen derived T cell epitope and provides an additional signal to the T cell through reduction of surface receptor. This supra-optimal activation results in T cells acquiring cytolytic properties for the cell presenting the T cell epitope, as well as suppressive properties on bystander T cells. In this way, the peptides or composition comprising the peptides described in the present invention, which contain an antigen-derived T cell epitope and, outside the epitope, a modified oxidoreductase motif can be used for direct immunisation of mammals, including human beings. The invention thus provides peptides of the invention or derivatives thereof, for use as a medicine. Accordingly, the present invention provides therapeutic methods which comprise administering one or more peptides according to the present invention to a patient in need thereof.

The present invention offers methods by which antigen-specific T cells endowed with cytolytic properties can be elicited by immunisation with small peptides. It has been found that peptides which contain (i) a sequence encoding a T cell epitope from an antigen and (ii) a consensus sequence with redox properties, and further optionally also comprising a sequence to facilitate the uptake of the peptide into late endosomes for efficient MHC-class Il or CD1d presentation, elicit suppressor T-cells.

The immunogenic properties of the peptides of the present invention are of particular interest in the treatment and prevention of immune reactions.

Peptides described herein are used as medicament, more particularly used for the manufacture of a medicament for the prevention or treatment of an immune disorder in a mammal, more in particular in a human.

The present invention describes methods of treatment of an immune disorder of a mammal in need for such treatment, by using the peptides of the invention, homologues or derivatives thereof, the methods comprising the step of administering to said mammal suffering or at risk of an immune disorder a therapeutically effective amount of the peptides of the invention, homologues or derivatives thereof such as to reduce the symptoms of the immune disorder. The treatment of both humans and animals, such as, pets and farm animals is envisaged. In an embodiment the mammal to be treated is a human. The immune disorders referred to above are in a particular embodiment selected from allergic diseases and autoimmune diseases. More particularly, such immunogenic peptides are provided for use in treating or alleviating symptoms of MS.

The peptides of the invention or the pharmaceutical composition comprising such as defined herein is preferably administered through sub-cutaneous or intramuscular administration. Preferably, the peptides or pharmaceutical compositions comprising such can be injected sub-cutaneously (SC) in the region of the lateral part of the upper arm, midway between the elbow and the shoulder. When two or more separate injections are needed, they can be administered concomitantly in both arms.

The peptide according to the invention or the pharmaceutical composition comprising such is administered in a therapeutically effective dose. Exemplary but non-limiting dosage regimens are between 50 and 1500 μg, preferably between 100 and 1200 μg. More specific dosage schemes can be between 50 and 250 μg, between 250 and 450 ug or between 850 and 1300 μg, depending on the condition of the patient and severity of disease. Dosage regimen can comprise the administration in a single dose or in 2, 3, 4, 5, or more doses, either simultaneously or consecutively. Exemplary non-limiting administration schemes are the following:
A low dose scheme comprising the SC administration of 50 μg of peptide in two separate injections of 25 μg each (100 μL each) followed by three consecutive injections of 25 μg of peptide as two separate injections of 12.5 μg each (50 μL each).
A medium dose scheme comprising the SC administration of 150 μg of peptide in two separate injections of 75 μg each (300 μL each) followed by three consecutive administrations of 75 μg of peptide as two separate injections of 37.5 μg each (150 μL each).
A high dose scheme comprising the SC administration of 450 μg of peptide in two separate injections of 225 μg each (900 μL each) followed by three consecutive administrations of 225 μg of peptide as two separate injections of 112.5 μg each (450 μL each).

Other exemplary non-limiting administration schemes are the following:
A dose scheme comprising 6 SC administration 2 weeks apart of 450 μg of peptide in two separate injections of 225 μg each.
A dose scheme comprising 6 SC administration 2 weeks apart SC of 1350 μg of peptide in two separate injections of 675 μg each.

A particularly but non-limiting dosage regimen of the immunogenic peptide as defined herein is between 50 and 1500 μg, preferably between 450 and 1500 μg. Dosage regimen can comprise the administration in a single dose or in 2, 3, 4, 5, 6 or more doses, either simultaneously or consecutively. Said treatment with the immunogenic peptide can be done 1 to 6 times, such as 1 to 4 times, preferably every 5 to 9 days, such as about every 7 days.

For all the above peptides additional variant are envisaged, wherein between the Histidine flanking residue and the first Cysteine of the oxidoreductase motif, one or two amino acids X are present. Typically these external amino acid(s) X is (are) not His, Cys, Ser or Thr.

The peptides of the present invention can also be used in diagnostic in vitro methods for detecting class Il restricted CD4 + T cells or NKT cells in a sample. In this method a sample is contacted with a complex of an MHC class Il or CD1d molecule and a peptide according to the present invention. The CD4+ T cells or NKT cells detected by measuring the binding of the complex with cells in the sample, wherein the binding of the complex to a cell is indicative for the presence of CD4 +T cells or NKT cells in the sample.

The complex can be a fusion protein of the peptide and an MHC class Il or CD1d molecule.

Alternatively MHC or CD1d molecules in the complex are tetramers. The complex can be provided as a soluble molecule or can be attached to a carrier.

Accordingly, in particular embodiments, the methods of treatment and prevention of the present invention comprise the administration of an immunogenic peptide as described herein, wherein the peptide comprise a T cell epitope of an antigenic protein which plays a role in the disease to be treated (for instance such as those described above). In further particular embodiments, the epitope used is a dominant epitope.

Peptides in accordance of the present invention will be prepared by synthesising a peptide wherein T cell epitope and modified oxidoreductase motif will be separated by 0 to 7 amino acids. In certain embodiments the modified oxidoreductase motif can be obtained by introducing 1, 2 or 3 mutations outside the epitope sequence, to preserve the sequence context as occurring in the protein. Typically amino-acids in P-2 and P-1, as well as in P+10 and P+11, with reference to the nonapeptide which are part of the natural sequence are preserved in the peptide sequence. These flanking residues generally stabilize the binding to MHC class Il or CD1d. In other embodiments the sequence N terminal or C terminal of the epitope will be unrelated to the sequence of the antigenic protein containing the T cell epitope sequence.

Thus based upon the above methods for designing a peptide, a peptide is generated by chemical peptide synthesis, recombinant expression methods or in more exceptional cases, proteolytic or chemical fragmentation of proteins.

Peptides as produced in the above methods can be tested for the presence of a T cell epitope in in vitro and in vivo methods, and can be tested for their reducing activity in in vitro assays. As a final quality control, the peptides can be tested in in vitro assays to verify whether the peptides can generate CD4+ T cells or NKT cells which are cytolytic via an apoptotic pathway for antigen presenting cells presenting the antigen which contains the epitope sequence which is also present in the peptide with the modified oxidoreductase motif.

The peptides of the present invention can be generated using recombinant DNA techniques, in bacteria, yeast, insect cells, plant cells or mammalian cells. In view of the limited length of the peptides, they can be prepared by chemical peptide synthesis, wherein peptides are prepared by coupling the different amino acids to each other. Chemical synthesis is particularly suitable for the inclusion of e.g. D-amino acids, amino acids with non-naturally occurring side chains, etc.

Chemical peptide synthesis methods are well described and peptides can be ordered from companies such as Applied Biosystems and other companies.

Peptide synthesis can be performed as either solid phase peptide synthesis (SPPS) or contrary to solution phase peptide synthesis. The best known SPPS methods are t-Boc and Fmoc solid phase chemistry:

During peptide synthesis several protecting groups are used. For example hydroxyl and carboxyl functionalities are protected by t-butyl group, lysine and tryptophan are protected by t-Boc group, and asparagine, glutamine, cysteine and histidine are protected by trityl group, and arginine is protected by the pbf group. If appropriate, such protecting groups can be left on the peptide after synthesis. Peptides can be linked to each other to form longer peptides using a ligation strategy (chemoselective coupling of two unprotected peptide fragments) as originally described by Kent (Schnelzer & Kent (1992) Int. J. Pept. Protein Res. 40, 180-193) and reviewed for example in Tam et al. (2001) Biopolymers 60, 194-205 provides the tremendous potential to achieve protein synthesis which is beyond the scope of SPPS. Many proteins with the size of 100-300 residues have been synthesised successfully by this method. Synthetic peptides have continued to play an ever increasing crucial role in the research fields of biochemistry, pharmacology, neurobiology, enzymology and molecular biology because of the enormous advances in the SPPS.

Alternatively, the peptides can be synthesised by using nucleic acid molecules which encode the peptides of this invention in an appropriate expression vector which include the encoding nucleotide sequences. Such DNA molecules may be readily prepared using an automated DNA synthesiser and the well-known codon-amino acid relationship of the genetic code. Such a DNA molecule also may be obtained as genomic DNA or as cDNA using oligonucleotide probes and conventional hybridisation methodologies. Such DNA molecules may be incorporated into expression vectors, including plasmids, which are adapted for the expression of the DNA and production of the polypeptide in a suitable host such as bacterium, e.g. Escherichia coli, yeast cell, animal cell or plant cell.

The physical and chemical properties of a peptide of interest (e.g. solubility, stability) are examined to determine whether the peptide is/would be suitable for use in therapeutic compositions. Typically this is optimised by adjusting the sequence of the peptide. Optionally, the peptide can be modified after synthesis (chemical modifications e.g. adding/deleting functional groups) using techniques known in the art.

T cell epitopes on their own are thought to trigger early events at the level of the T helper cell by binding to an appropriate HLA molecule on the surface of an antigen presenting cell and stimulating the relevant T cell subpopulation. These events lead to T cell proliferation, lymphokine secretion, local inflammatory reactions, the recruitment of additional immune cells to the site, and activation of the B cell cascade leading to production of antibodies. One isotype of these antibodies, IgE, is fundamentally important in the development of allergic symptoms and its production is influenced early in the cascade of events, at the level of the T helper cell, by the nature of the lymphokines secreted. A T cell epitope is the basic element or smallest unit of recognition by a T cell receptor where the epitope comprises amino acid residues essential to receptor recognition, which are contiguous in the amino acid sequence of the protein.

However, upon administration of the peptides with a T-cell epitope and an oxidoreductase motif, the following events are believed to happen:
activation of antigen (i) specific T cells resulting from cognate interaction with the antigen-derived peptide presented by MHC-class Il molecules;
the reductase sequence reduces T cell surface proteins, such as the CD4 molecule, the second domain of which contains a constrained disulfide bridge. This transduces a signal into T cells. Among a series of consequences related to increased oxidative pathway, important events are increased calcium influx and translocation of the NF-KB transcription factor to the nucleus. The latter results in increased transcription of IFN-gamma and granzymes, which allows cells to acquire cytolytic properties via an apoptosis-inducing mechanism; the cytolytic property affects cells presenting the peptide by a mechanism, which involves granzyme B secretion, and Fas-FasL interactions. Since the cell killing effect is obtained via an apoptotic pathway, cytolytic cells is a more appropriate term for these cells than cytotoxic cells. Destruction of the antigen-presenting target cells prevents activation of other T cells specific for epitopes located on the same antigen, or to an unrelated antigen that would be processed by the same antigen-presenting cell; an additional consequence of T cell activation is to suppress activation of bystander T cells by a cell-cell contact dependent mechanism.

In such a case, T cells activated by an antigen presented by a different antigen-presenting cell is also suppressed provided both cytolytic and bystander T cells are in close proximity, namely activated on the surface of the same antigen-presenting cell. The above-postulated mechanism of action is substantiated with experimental data disclosed in the above cited PCT application WO2008/017517 and publications of the present inventors.

Similarly, NKT cell epitopes will reduce the immune response according to the following mechanism, as postulated and shown in WO2012/069568 and publications of the present inventors. When NKT cells are activated by a peptide modified as to contain a thioreductase activity, the latter increases significantly the properties of NKT cells and thereby increases the killing of cells carrying autoantigens by antigen-specific CD4+NKT cells, which suppresses the immune response against said autoantigens. The participation of NKT cells in the control of immune responses in auto-immune diseases, or against allofactors or allergens has been reported on a number of occasions (Jahng et al Journal of experimental Medicine 199: 947-957, 2004; Van Belle and von Herrath, Molecular Immunology 47: 8-1 1, 2009) but relatively difficult to describe. In WO2012/069568, it was shown that peptides can be presented by the CD1d molecule. A characteristic of the CD1d molecule is to be made of 2 anti-parallel alpha chains forming a cleft sitting atop of a platform made of two anti-parallel beta chains. The cleft is narrow and deep and accepts only hydrophobic residues, classically deemed to be only lipids. Peptides with hydrophobic residues have the capacity to bind to the CD1d cleft. Besides, as the cleft is open both sides, peptides longer than 7 amino acids can be accommodated. Hydrophobic peptides carrying the CD1d motif are often found in autoantigens, allofactors and allergens, thereby endowing said autoantigen, allofactor or allergen with the capacity to activate CD4+ NKT cells. Direct elimination by killing of cells presenting said autoantigen, allofactor or allergen eliminates the capacity to mount an immune response against these antigens/factors.

The present invention relates to the production of peptides containing hydrophobic residues derived from AQP4 that confer the capacity to bind to the CD1d molecule. Upon administration, such peptides are taken up by APC, directed to the late endosome where they are loaded onto CD1d and presented at the surface of the APC. Said hydrophobic AQP4 peptides being characterized by a motif corresponding to the general sequence [FWHY]-XX-[ILMV]-XX-[FWTHY] (SEQ ID NO: 316) or [FW]-XX-[ILMV]-XX-[FW] (SEQ ID NO: 317), in which positions P1 and P7 are occupied by hydrophobic residues such as phenylalanine (F) or tryptophan (W). P7 is however permissive in the sense that it accepts alternative hydrophobic residues to phenylalanine or tryptophan, such as threonine (T) or histidine (H). The P4 position is occupied by an aliphatic residue such as isoleucine (I), leucine (L) or methionine (M). The present invention provides methods for generating antigen-specific cytolytic CD4+ T cells either in vivo or in vitro and, independently thereof, methods to discriminate cytolytic CD4+ T cells from other cell populations such as Foxp3+ Tregs based on characteristic expression data.

The present invention describes in vivo methods for the production of the antigen-specific CD4+ T cells. A particular embodiment relates to the method for producing or isolating the CD4+ T cells by immunising animals (including humans) with the peptides of the invention as described herein and then isolating the CD4+ T cells from the immunised animals. The present invention describes in vitro methods for the production of antigen specific cytolytic CD4+ T cells towards APC. The present invention provides methods for generating antigen specific cytolytic CD4 +T cells towards APC.

In one embodiment, methods are provided which comprise the isolation of peripheral blood cells, the stimulation of the cell population in vitro by an immunogenic peptide according to the invention and the expansion of the stimulated cell population, more particularly in the presence of IL-2. The methods according to the invention have the advantage a high number of CD4+ T cells is produced and that the CD4+ T cells can be generated which are specific for the antigenic protein (by using a peptide comprising an antigen-specific epitope).

In an alternative embodiment, the CD4+ T cells can be generated in vivo, i.e. by the injection of the immunogenic peptides described herein to a subject, and collection of the cytolytic CD4+ T cells generated in vivo.

The antigen-specific cytolytic CD4+ T cells towards APC, obtainable by the methods of the present invention are of particular interest for the administration to mammals for immunotherapy, in the prevention of allergic reactions and the treatment of auto-immune diseases. Both the use of allogenic and autogeneic cells are envisaged. Cytolytic CD4+ T cells populations are obtained as described herein below. Antigen-specific cytolytic CD4+ T cells as described herein can be used as a medicament, more particularly for use in adoptive cell therapy, more particularly in the treatment of acute allergic reactions and relapses of autoimmune diseases such as multiple sclerosis. Isolated cytolytic CD4+ T cells or cell populations, more particularly antigen-specific cytolytic CD4+ T cell populations generated as described are used for the manufacture of a medicament for the prevention or treatment of immune disorders.

Methods of treatment by using the isolated or generated cytolytic CD4+ T cells are disclosed.

The peptides of the invention will, upon administration to a living animal, typically a human being, elicit specific T cells exerting a suppressive activity on bystander T cells. In specific embodiments the cytolytic cell populations of the present invention are characterised by the expression of FasL and/or Interferon gamma. In specific embodiments the cytolytic cell populations of the present invention are further characterised by the expression of GranzymeB.

This mechanism also implies and the experimental results show that the peptides of the invention, although comprising a specific T-cell epitope of a certain antigen, can be used for the prevention or treatment of disorders elicited by an immune reaction against other T-cell epitopes of the same antigen or in certain circumstances even for the treatment of disorders elicited by an immune reaction against other T-cell epitopes of other different antigens if they would be presented through the same mechanism by MHC class Il molecules in the vicinity of T cells activated by peptides of the invention. Isolated cell populations of the cell type having the characteristics described above, which, in addition are antigen-specific, i.e. capable of suppressing an antigen-specific immune response are disclosed.

The present invention provides pharmaceutical compositions comprising one or more peptides according to the present invention, further comprising a pharmaceutically acceptable carrier. As detailed above, the present invention also relates to the compositions for use as a medicine or to methods of treating a mammal of an immune disorder by using the composition and to the use of the compositions for the manufacture of a medicament for the prevention or treatment of immune disorders. The pharmaceutical composition could for example be a vaccine suitable for treating or preventing immune disorders, especially airborne and foodborne allergy, as well as diseases of allergic origin. As an example described further herein of a pharmaceutical composition, a peptide according to the invention is adsorbed on an adjuvant suitable for administration to mammals, such as aluminium hydroxide (alum). Typically, 50 μg of the peptide adsorbed on alum are injected by the subcutaneous route on 3 occasions at an interval of 2 weeks. It should be obvious for those skilled in the art that other routes of administration are possible, including oral, intranasal or intramuscular. Also, the number of injections and the amount injected can vary depending on the conditions to be treated. Further, other adjuvants than alum can be used, provided they facilitate peptide presentation in MHC-class Il or CD1d presentation and T cell activation. Thus, while it is possible for the active ingredients to be administered alone, they typically are presented as pharmaceutical formulations. The formulations, both for veterinary and for human use, of the present invention comprise at least one active ingredient, as above described, together with one or more pharmaceutically acceptable carriers. The present invention relates to pharmaceutical compositions, comprising, as an active ingredient, one or more peptides according to the invention, in admixture with a pharmaceutically acceptable carrier. The pharmaceutical composition of the present invention should comprise a therapeutically effective amount of the active ingredient, such as indicated hereinafter in respect to the method of treatment or prevention. Optionally, the composition further comprises other therapeutic ingredients. Suitable other therapeutic ingredients, as well as their usual dosage depending on the class to which they belong, are well known to those skilled in the art and can be selected from other known drugs used to treat immune disorders.

The immunogenic peptide as defined herein may be adsorbed on an adjuvant suitable for administration to mammals, such as aluminium hydroxide (alum). Typically, 50 μg of the peptide adsorbed on alum are injected by the subcutaneous route on 3 occasions at an interval of 2 weeks. It should be obvious for those skilled in the art that other routes of administration are possible, including, but not limited to, oral, intranasal or intramuscular. Also, the number of injections and the amount injected can vary depending on the severity of the condition to be treated, and other parameters, such as the age, body weight, general health, sex and diet of the patient. Further, other adjuvants than alum can be used, provided they facilitate peptide presentation in MHC-class Il or CD1d and T or NKT cell activation. Thus, while it is possible for the immunogenic peptides to be administered without any adjuvant, they typically are presented as pharmaceutical formulations. The formulations, both for veterinary and for human use, comprise at least one immunogenic peptide, as above described, together with one or more pharmaceutically acceptable carriers.

The term “pharmaceutically acceptable carrier” as used herein with respect to the immunogenic peptide as defined herein means any material or substance with which the immunogenic peptide is formulated in order to facilitate its application or dissemination to the locus to be treated, for instance by dissolving, dispersing or diffusing the composition, and/or to facilitate its storage, transport or handling without impairing its effectiveness. They include any and all solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the immunogenic peptide in the pharmaceutical formulation. The pharmaceutically acceptable carrier may be a solid or a liquid or a gas which has been compressed to form a liquid, i.e. the formulations can suitably be used as concentrates, emulsions, solutions, granulates, dusts, sprays, aerosols, suspensions, ointments, creams, tablets, pellets or powders. Suitable pharmaceutical carriers for use in the pharmaceutical formulations of the peptide are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical formulations of the immunogenic peptide may be prepared in any known manner, for instance by homogeneously mixing, coating and/or grinding the active ingredients, in a one- step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the immunogenic peptide.

Suitable surface-active agents for use in the pharmaceutical formulations of the immunogenic peptide, also known as emulgent or emulsifier, non-ionic, cationic and/or anionic materials having good emulsifying, dispersing and/or wetting properties. Suitable anionic surfactants include both water- soluble soaps and water-soluble synthetic surface-active agents. Suitable soaps are alkaline or alkaline-earth metal salts, unsubstituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives typically contain 8 to 22 carbon atoms. Examples of alkylarylsulphonates are the sodium, calcium or alcanolamine salts of dodecyl benzene sulphonic acid or dibutyl-naphtalenesulphonic acid or a naphtalene-sulphonic acid/formaldehyde condensation suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidyl-ethanolamine, phosphatidylserine, phosphatidylglycerine, lysolecithin, cardio- lipin, dioctanylphosphatidylcholine, dipalmitoylphoshatidylcholine and their mixtures. Suitable non-ionic surfactants include polyethoxylated and poly-propoxylated derivatives of alkyl phenols, fatty alcohols, fatty acids, aliphatic amines or amides containing at least 12 carbon atoms in the molecule, alkylarene sulphonates and dialkylsulphosuccinates, such as polyglycol ether derivatives of aliphatic and cycloaliphatic alcohols, saturated and unsaturated fatty acids and alkylphenols, the derivatives typically containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediamino- polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants. Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C&C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

The pharmaceutical dosage forms or pharmaceutical formulations of the immunogenic peptide suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the immunogenic peptide in the required amount in the appropriate solvent with various of the other ingredients enumerated above, as required, followed by filtered sterilization. Generally, dispersions are prepared by incorporating the sterilized immunogenic peptide into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the immunogenic peptide plus any additional desired ingredient from a previously sterile-filtered solution thereof.

Upon formulation, pharmaceutical preparations as defined herein or the peptides as defined herein or the fumarate compound as defined herein can be administered in a manner compatible with the dosage formulation and in such amount as is therapeutically effective.

The peptides of the invention or the pharmaceutical composition comprising such as defined herein is preferably administered through sub-cutaneous or intramuscular administration. Preferably, the peptides or pharmaceutical compositions comprising such can be injected sub-cutaneously (SC) in the region of the lateral part of the upper arm, midway between the elbow and the shoulder. When two or more separate injections are needed, they can be administered concomitantly in both arms.

The peptide according to the invention or the pharmaceutical composition comprising such is administered in a therapeutically effective dose. Exemplary but non-limiting dosage regimens are between 50 and 1500 μg, preferably between 100 and 1200 μg. More specific dosage schemes can be between 50 and 250 μg, between 250 and 450 ug or between 850 and 1300 μg, depending on the condition of the patient and severity of disease. Dosage regimen can comprise the administration in a single dose or in 2, 3, 4, 5, or more doses, either simultaneously or consecutively.

In certain embodiments, the treatment can be repeated several times throughout the disease of the subject. Such consecutive treatments can be done daily, or with an intermission of 1 to 10 days, such as for example every 5 to 9 days such as about every 7 days.

Alternatively, said treatment can be repeated weekly, biweekly, monthly, bimonthly, or every three to four months.

Exemplary non-limiting administration schemes are the following:
A low dose scheme comprising the SC administration of 50 μg of peptide in two separate injections of 25 μg each (100 μL each) followed by three consecutive injections of 25 μg of peptide as two separate injections of 12.5 μg each (50 μL each).
A medium dose scheme comprising the SC administration of 150 μg of peptide in two separate injections of 75 μg each (300 μL each) followed by three consecutive administrations of 75 μg of peptide as two separate injections of 37.5 μg each (150 μL each).
A high dose scheme comprising the SC administration of 450 μg of peptide in two separate injections of 225 μg each (900 μL each) followed by three consecutive administrations of 225 μg of peptide as two separate injections of 112.5 μg each (450 uL each).

Other exemplary non-limiting administration schemes are the following:
A dose scheme comprising 6 SC administration 2 weeks apart of 450 μg of peptide in two separate injections of 225 μg each.
A dose scheme comprising 6 SC administration 2 weeks apart SC of 1350 μg of peptide in two separate injections of 675 μg each.

Other exemplary non-limiting administration schemes are the following:
A dose scheme comprising 6 SC administration 2 weeks apart of 450 μg of peptide in two separate injections of 225 μg each.
A dose scheme comprising 6 SC administration 2 weeks apart SC of 1350 μg of peptide in two separate injections of 675 μg each.

In a preferred embodiment said immunogenic peptide is administered in at least 5 doses of from 300 to 1500 μg of said immunogenic peptide with an interval of from about 12 days to about 28 days between two doses, preferably said administration is done through intramuscular or subcutaneous injection.

The immunogenic peptide for use according to aspect 1, wherein said immunogenic peptide is administered through intramuscular or subcutaneous injection of 6 doses of from 300 to 1500 μg of said immunogenic peptide with an interval of about 12 to 28 days between two doses.

Preferably, each of said doses of from 300 to 1500 μg of said immunogenic peptide is administered with an interval of about 12 to about 16 days, or about 2 weeks between two doses.

Preferably each dose contains:
from 300 to 600 μg of said immunogenic peptide;
from 600 to 800 μg of said immunogenic peptide;
from 800 to 1000 μg of said immunogenic peptide;
from 1000 to 1200 μg of said immunogenic peptide; or
from 1200 to 1500 μg of said immunogenic peptide.

In some preferred embodiments, a boost administration is performed of a dose of from 300 to 1500 μg of said immunogenic peptide at about week 22 to 30, counted from the start of the treatment. More preferably, said boost administration can be performed at about week 22 to 26 counted from the start of the treatment.

In preferred embodiments said boost contains:
from 300 to 600 μg of said immunogenic peptide;
from 600 to 800 μg of said immunogenic peptide;
from 800 to 1000 μg of said immunogenic peptide;
from 1000 to 1200 μg of said immunogenic peptide; or
from 1200 to 1500 μg of said immunogenic peptide.

The immunogenic peptide formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. The immunogenic peptide can be administered concomitantly in two sites (both upper arms, preferably in the region of the lateral part of the arms, more preferably midway between the elbow and the shoulder).

Other pharmaceutically acceptable forms of the immunogenic peptide can be readily envisaged by the skilled person.

Peptides, homologues or derivatives thereof according to the invention (and their physiologically acceptable salts or pharmaceutical compositions all included in the term “active ingredients”) may be administered by any route appropriate to the condition to be treated and appropriate for the compounds, here the proteins and fragments to be administered. Possible routes include regional, systemic, oral (solid form or inhalation), rectal, nasal, topical (including ocular, buccal and sublingual), vaginal and parenteral (including subcutaneous, intramuscular, intravenous, intradermal, intra-arterial, intrathecal and epidural). The preferred route of administration may vary with for example the condition of the recipient or with the diseases to be treated. As described herein, the carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intraarterial, intrathecal and epidural) administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Typical unit dosage formulations are those containing a daily dose or unit daily sub-dose, as herein above recited, or an appropriate fraction thereof, of an active ingredient. It should be understood that in addition to the ingredients particularly mentioned above the formulations of this invention may include other agents conventional in the art having regard to the type of formulation in question, for example those suitable for oral administration may include flavouring agents. Peptides, homologues or derivatives thereof according to the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, microspheres, microemulsions, nanoparticles, nanocapsules and so on. Depending on the route of administration, the pharmaceutical composition may require protective coatings. Pharmaceutical forms suitable for injection include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof. In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments.

In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

Cytolytic CD4+T cells as obtained in the present invention, induce APC apoptosis after MHC-class Il dependent cognate activation, affecting both dendritic and B cells, as demonstrated in vitro and in vivo, and (2) suppress bystander T cells by a contact-dependent mechanism in the absence of IL-10 and/or TGF-beta. Cytolytic CD4+ T cells can be distinguished from both natural and adaptive Tregs, as discussed in detail in WO2008/017517.

Similarly, NKT cells as obtained in the present invention, i.e. activated by a AQP4-derived peptide according to the invention containing a thioreductase activity, the latter increases significantly the properties of NKT cells and thereby increases the killing of cells carrying AQP4 autoantigens by antigen-specific CD4+ NKT cells, which suppresses the immune response against said AQP4 autoantigens. This mechanism is discussed in detail in WO2012/069568.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples.

### EXAMPLES

### Example 1: Peptide Design

Immunogenic peptides comprising an oxidoreductase motif CPYC (SEQ ID NO: 157), more particularly with the sequence HCPYC (SEQ ID NO: 158) or KHCPYC (SEQ ID NO: 159) linked to an HLA-DRB1*03:01 and/or HLA-DPB1*05:01 human T cell epitope of Aquaporin 4 (AQP4) were designed and synthesized (Table 1). All the peptides comprise a natural human AQP4 epitope or a variant with a serine (S) instead of the cysteine (C).

TABLE 1
List of immunogenic peptides

| Seq. | Peptide | N- | Redox | | | | Region |
|---|---|---|---|---|---|---|---|
| ID# | # | Term | motif | Linker* | T epitope* | C-term* | AQP4 |
| 137 | P1 | H | CPYC | REN | IMVAFKGVW | TQAK | 19-33 |
| 138 | P2 | KH | CPYC | KP | LPVDMVLIS | LSFK | 64-77 |
| 139 | P3 | H | CPYC | TVA | MVSTRKISI | AK | 101-114 |
| 140 | P4 | KH | CPYC | AVTVA | MVSTRKISI | AKKK | 101-114 |
| 141 | P5 | KH | CPYC | AVTVA | MVCTRKISI | AKKK | 101-114 |
| 142 | P6 | H | CPYC | AVTVA | MVCTRKISI | AKK | 101-114 |
| 143 | P7 | H | CPYC | TRK | ISIAKSVFY | IAA | 107-121 |
| 144 | P10 | H | CPYC | TRK | ISIAKSVFY | IAAKK | 107-121 |
| 145 | P11 | KH | CPYC | TRK | ISIAKSVFY | IAAKK | 107-121 |
| 146 | P12 | H | CPYC | TRK | ISIAKSVFY | IAAKKK | 107-121 |
| 147 | P13 | H | CPYC | LVE | LIITFQLVF | TIDD | 161-174 |
| 148 | P14 | KH | CPYC | LVE | LIITFQLVF | TIK | 161-174 |
| 149 | P15 | H | CPYC | VFT | IFASSDSKR | TDVK | 171-185 |
| 150 | P16 | H | CPYC | VFT | IFASCDSKR | TDVK | 171-185 |
| 151 | P17 | H | CPYC | VFT | IFASCDSKR | TDVKK | 171-185 |
| 152 | P18 | KH | CPYC | VFT | IFASCDSKR | TDVK | 171-185 |
| 153 | P19 | KH | CPYC | LFA | INYTGASMN | PAR | 202-216 |
| 154 | P20 | H | CPYC | EYV | FSPDVEFKR | RFK | 249-263 |
| 155 | P21 | H | CPYC | EYV | FCPDVEFKR | RFK | 249-263 |
| 156 | P22 | H | CPYC | RSQ | VETDDLILK | PGVB | 284-298 |

*Residues in bold are added residues that do not naturally occur at that position in the amino acid sequence of the AQP4 auto-antigen; underlined residues are mutated versus the wild-type amino acid sequence of the AQP4 auto-antigen.

### Example 2: Assessment of the Oxidoreductase Activity of the Immunogenic Peptides

The oxidoreductase activity of the immunogenic peptides of table 1 was determined using a fluorescent assay described in Tomazzolli et al. (2006) Anal. Biochem. 350, 105-112. Two peptides with a FITC label become self-quenching when they form a covalent disulfide bond. Upon reduction by a peptide in accordance with the present invention, the reduced individual FITC labelled peptides emit fluorescence again. The activity is expressed as the mean of duplicates. The results are expressed in Relative Fluorescent Units (RFU).

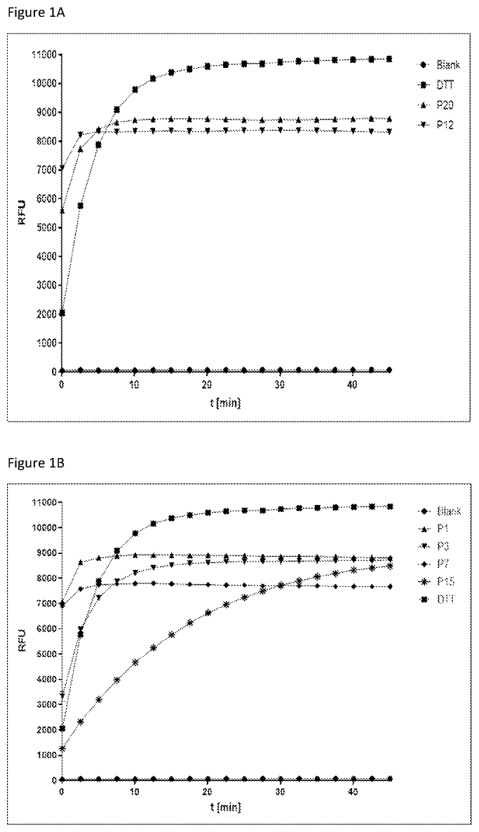

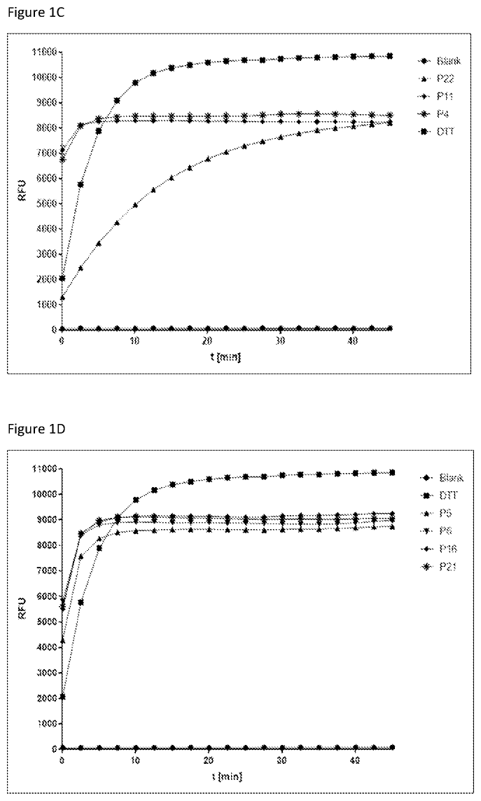

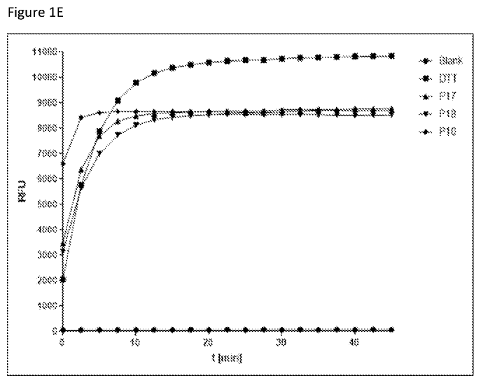

Peptides P12 and P20, used in example 4 for the generation of cytolytic CD4+ T cells, have a similar oxidoreductase activity that increased rapidly and reached a plateau after 5 min (FIG. 1A). Other peptides of table 1 were also tested and it is shown they all displayed oxidoreductase activity, although to a lesser extend as far as P15 and P22 were concerned (FIGS. 1B to 1E).

### Example 3: Assessment of the Binding Activity of the Immunogenic Peptides to Soluble HLA-DRB1*03:01 and HLA-DPB1*05:01 Proteins

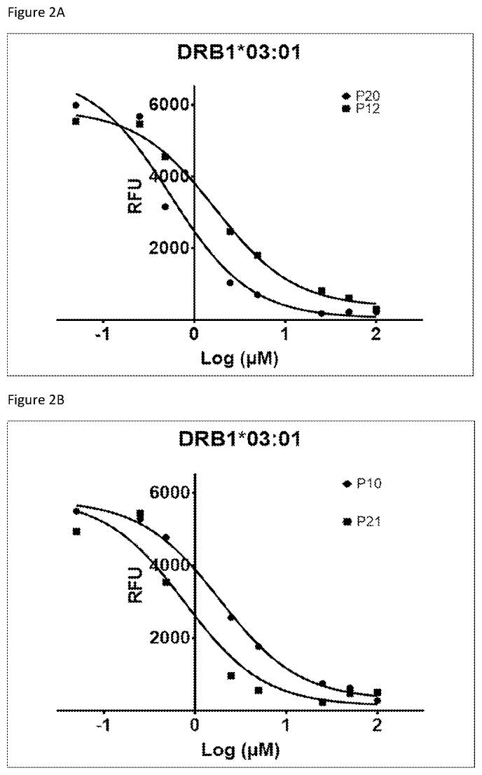

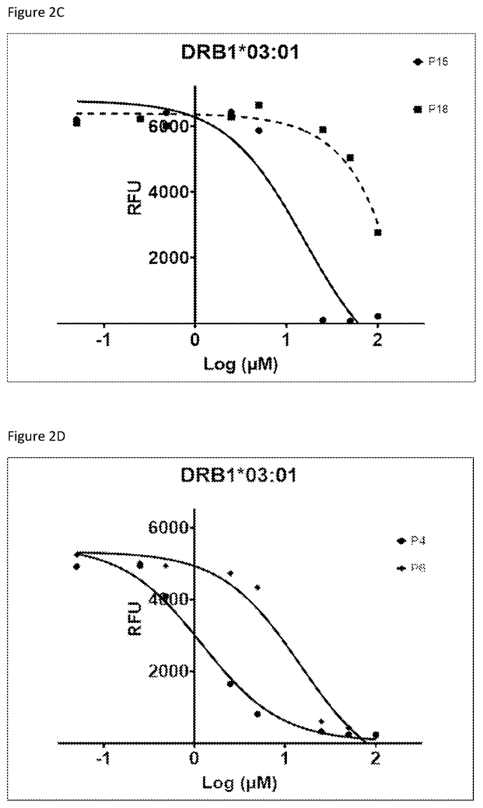

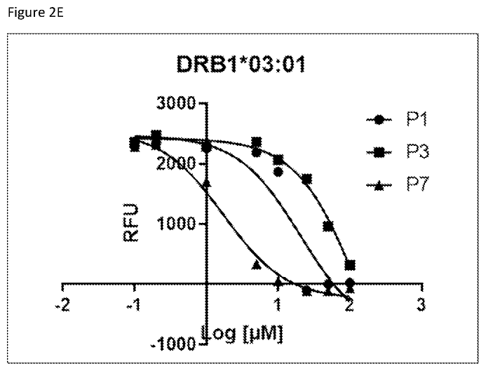

To test the binding of the immunogenic peptides of table 1 to MHCII molecules, a soluble phase competition assay is performed. The increasing concentrations of the peptides compete with biotin-labelled control peptide (high affinity binder of the corresponding MHCII molecule, Eurogentec, Seraing, Belgium) for binding to the soluble HLA-DRB1*03:01 (also named DR3) human MHC II protein (purchased from the Benaroya Research Institute, Seattle, US). As binding approaches its equilibrium (18h), biotin-labelled peptide/MHC II complexes are captured, separated from unbound reagents, and quantitatively detected by time-resolved fluorescence (Eu³⁺ streptavidin, Perkin Elmer, Brussels, Belgium). Since the biotinylated control peptide is responsible for the fluorescence signal (Eu³⁺ streptavidin/biotin interaction), the decrease in fluorescence intensity reflects the binding of tested peptides. Data are processed and plotted to ascertain dose-dependent binding properties of the peptides. All the tests are performed in triplicates. We shown that the peptides P12 and P20, used in example 4 for the generation of cytolytic CD4+ T cells, bind to HLA-DRB1*03:01 with similar affinity (FIG. 2A). Other peptides of table 1 were also tested and it is shown they all bind to HLA-DRB1*03:01, although with less affinity than P12 and P20 in as far as P1, P3, P6, P15 and P18 were concerned (FIGS. 2B to 2G).

Binding of the peptides of table 1 to soluble HLA-DPB1*05:01 protein is also assessed.

### Example 4: Ability of the Immunogenic Peptides to Induce Specific CD4+ T Cells with Lytic Properties

### Materials and Methods

The haplotype of the patients used in this study is shown in table 2 below.

TABLE 2
Haplotype of the patients included in the present study:

Tested

| patients | Haplotype | | | |
|---|---|---|---|---|
| NMO-001 | HLA | HLA | HLA | HLA |
| | DRB1*03:01 | DRB1*12:01 | DPB1*04:01 | DPB1*04:02 |
| NMO-003 | HLA | HLA | HLA | HLA |
| | DRB1*03:01 | DRB1*08:04 | DPB1*01:01 | DPB1*04:01 |
| NMO-006 | HLA | HLA | HLA | HLA |
| | DRB1*03:01 | DRB1*04:05 | DPB1*03:01 | DPB1*45:01 |

PBMCs are isolated from blood samples of patients with NMO on Lymphoprep density gradients. CD14+ monocytes are isolated from these PBMCs by performing positive immunomagnetic separation with CD14 microbeads (Miltenyi Biotec, 130-050-201) according to the supplier recommendations. CD14+ monocytes are differentiated for six days and maturated to generate autologous dendritic cells (mDC). CD19+ B cells are isolated from the CD14− PBMCs fraction by performing positive immunomagnetic separation with CD19 microbeads (Miltenyi Biotec, 130-050-301) according to the supplier recommendations. CD19+B cells are cultured and immortalized with EBV to generate autologous lymphoblastoid cell lines (LCL). Naïve CD4+ T cells are also purified from the CD14− PBMCs fraction by performing negative immunomagnetic separation with naïve CD4+ T cell isolation kit (Miltenyi Biotec, 130-094-131) according to the supplier recommendations. Naïve CD4+ T cells are co-cultured with autologous mDC or LCL in the presence of the peptides of table 1. The CD4+ T cells are re-stimulated periodically, about every 10-12 days, to generate peptide-specific cell lines.

The evaluation of the ability of the peptides of table 1 to generate peptide specific CD4+ T cells is evaluated by flow cytometry analysis of the TCR induced surface activation marker CD154 expression after overnight co-culture at resting state with autologous

LCL without (no peptide) or with the peptides of table 1. The expression of the lytic marker Granzyme B is also evaluated by flow cytometry analysis in the supernatant of overnight co-culture with autologous LCL without (no peptide) or with the peptides of table 1. Supernatants are analyzed with a BioLegend kit according to the supplier recommendations.

The ability of the peptides of table 1 to induce cytokines secretion in CD4+ T cells culture supernatants is evaluated by flow cytometry analysis after overnight co-culture at resting state with autologous mDC or LCL without (no peptide) or with the peptides of table 1. Supernatants are analyzed with the LEGENDplex Human Th Panel (13-plex) (BioLegend, 740721) according to the supplier recommendations.

The cytolytic activity of the peptide specific CD4+ T cells is evaluated by quantifying the apoptosis induced on LCL used as antigen presenting cells. Fluorescently labelled autologous LCL, loaded or not with the peptides, were overnight co-cultured at resting state with peptide specific CD4+ T cells, and LCL apoptosis was quantified by flow cytometry through Annexin V staining. Considering the apoptosis percentage of unloaded LCL, used as control, the percentage of specific apoptosis is calculated as follows:

$$\frac{\% Annexin V + of loaded LCL - \% Annexin V + of unloaded LCL}{100 - \% Annexin V + of unloaded LCL} \times 100$$

Results with P20

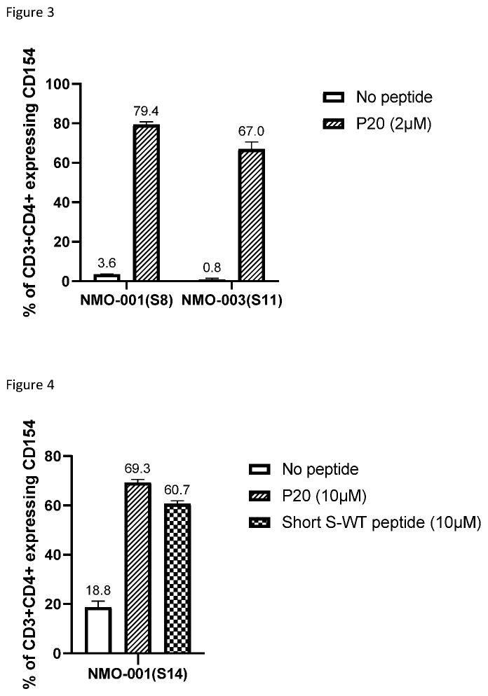

CD4+ T cell lines specific for the P20 peptide of table 1 were generated. We shown that multiple stimulations of naïve CD4+ T cells of NMO patients -001 and -003 induced P20-specific CD4+ T cell lines with a high frequency of effector cells (CD3+CD4+CD154+, FIG. 3).

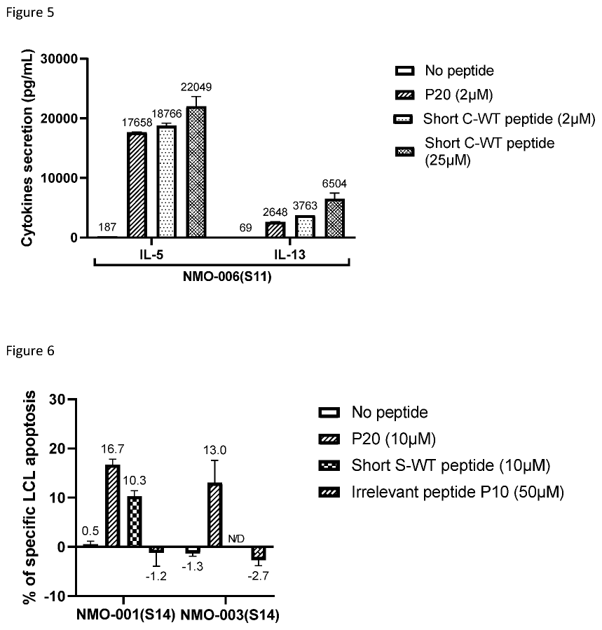

P20-specific CD4+ T cells of NMO-001 patient could also be reactivated by the corresponding short-S-WT epitope, comprising a serine instead of a cysteine in its sequence (AGGLYEYVFSPDVEFKRRFK, SEQ ID NO:397, FIG. 4). Moreover, a specific secretion of cytokines (IL-5 and IL-13) induced by P20 or the corresponding natural short C-WT T-cell epitope (sequence: AGGLYEYVFCPDVEFKRRFK, SEQ ID NO: 398) in culture supernatant of a P20-CD4+ cell lines generated from patient NMO-006 was observed, confirming the antigen specificity of this cell line (FIG. 5).

This also indicates that P20-specific CD4+ T cells are able to cross-react with APCs presenting the corresponding WT AQP4 epitope sequences.

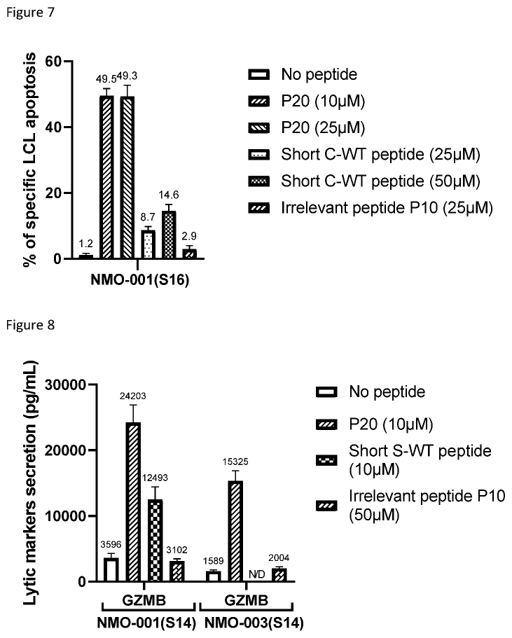

An increase in the percentage of specific LCL apoptosis when labelled autologous LCL, loaded with P20 or the corresponding short S WT T-cell epitope, are co-cultured with the P20-specific CD4+ T cell lines from patient NMO-001 (after 14 stimulations) is observed, further demonstrating the lytic activity of this P20-induced CD4+ T cell line (FIG. 6). Apoptosis of LCL loaded with P20 was also observed upon co-culture with the P20-specific CD4+ T cell lines from patient NMO-003 after 14 stimulations (FIG. 6). LCL apoptosis induced by co-culture with CD4+ T cells of patient NMO-001 after 16 stimulations with P20 reached 50% (FIG. 7). Cross-reactivity with short C WT T-cell epitope was however lower in this case (FIG. 7).

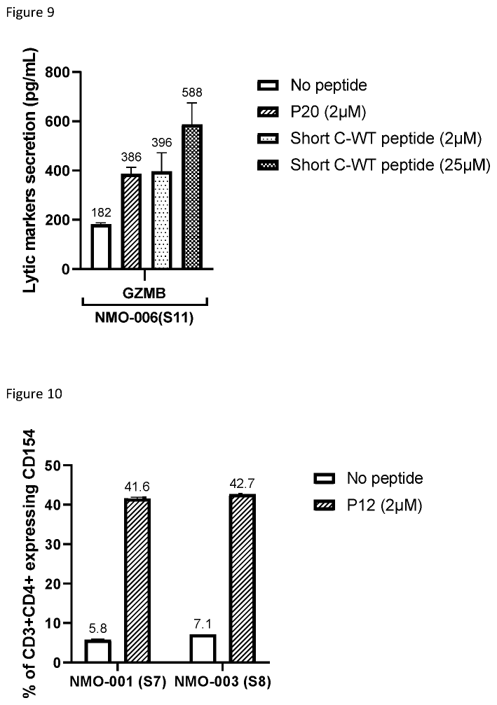

Finally, it was also shown that stimulation with P20 or the corresponding short C or S-WT T-cell epitope induced a specific increase of effector cells expressing the lytic marker Granzyme B (FIGS. 8 and 9).

Altogether, these results demonstrate that P20 is able to induce specific CD4+ T cells with lytic properties called cytolytic CD4+ T cells.

Results with P12

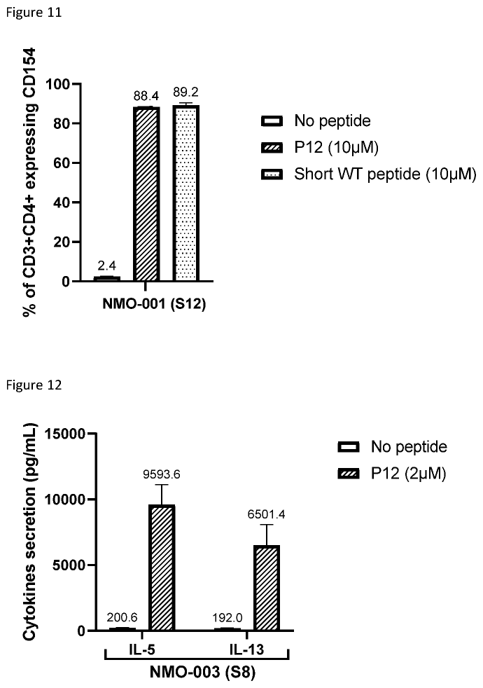

Multiple stimulations of naïve CD4+ T cells from NMO patients -001 and -003 with P12 induced P12-specific CD4+ T cell lines with a high frequency of effector cells (CD3+CD4+CD154+, FIG. 10). P12-specific CD4+ T cells of NMO-001 patient could also be reactivated by its corresponding short-WT epitope, (KVAMVCTRKISIAKSVFYIAAKK, SEQ ID NO:, SEQ ID NO: 399, FIG. 11). A specific secretion of cytokines (IL-5 and IL-13) induced by P12 in culture supernatant of the P12-specific CD4+cell lines was also observed (FIG. 12).

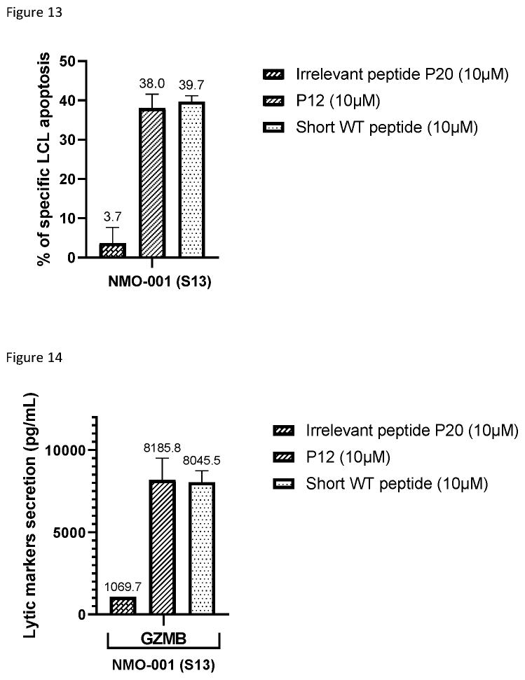

An increase in the percentage of specific LCL apoptosis when labelled autologous LCL, loaded with P12 or the corresponding short WT T-cell epitope, are co-cultured with the P12-specific CD4+ T cell lines from patients NMO-001 was observed (FIG. 13), demonstrating the lytic activity of the P12-induced CD4+ T cells and further indicating that P12-specific CD4+ T cells are able to cross-react with APCs presenting the corresponding WT AQP4 epitope sequence.

Finally, it was also shown that stimulation with P12 or its corresponding short WT T-cell epitope induced a specific increase of effector cells expressing the lytic marker Granzyme B (FIGS. 14).

Altogether, these results demonstrate that P12 is able to induce specific CD4+ T cells with lytic properties called cytolytic CD4+ T cells.

### Example 5: Effect of the Administration of the Peptides on anti-AQP4 Antibodies Production in Mice

To demonstrate that treatment with peptides of table 1 can reduce the antibody response against AQP4, a murine serology model is used. In this study, C57BL/6 mice are immunized twice, 35 days apart, with 10 μg of an AQP4 long peptide emulsified first in CFA, then in IFA. Between the two long peptide injections, C57BL/6 mice are immunized 4 times, 7 days apart, with 100 μg of the peptides of table 1 in the presence of alum. Control mice receives alum alone. Blood is collected at different time points during the study (before and after immunization) in order to determine the production kinetics of IgG against AQP4. The quantification of anti-AQP4 lgG is performed by ELISA using coated biotinylated versions of the AQP4 long peptide and mouse monoclonal anti AQP4 antibody. It is shown that the peptides of table 1 are able to reduce anti-AQP4 lgG production induced by the AQP4 long peptide.

SEQUENCES

SEQ ID NO: 322; Length: 7; Artificial Sequence; synthetic peptide
VARIANT (1)..(1)): X = F, W, H, or Y
VARIANT (2)..(3)): Xaa can be any naturally occurring amino acid
VARIANT (4)..(4)): X = I, L, M, or V
VARIANT (5)..(6)): Xaa can be any naturally occurring amino acid
VARIANT (7)..(7)): X = F, W, H, or Y
Xaa Xaa Xaa Xaa Xaa Xaa Xaa
SEQ ID NO: 323; Length: 4; Artificial Sequence; synthetic peptide
VARIANT (1)..(1): C, S, or T
VARIANT (2)..(3): Xaa can be any naturally occurring amino acid
Xaa Xaa Xaa Cys
SEQ ID NO: 324; Length: 5; Artificial Sequence; synthetic peptide
VARIANT (1)..(1): C, S, or T
VARIANT (2)..(4): Xaa can be any naturally occurring amino acid
Xaa Xaa Xaa Xaa Cys
SEQ ID NO: 325; Length: 6; Artificial Sequence; synthetic peptide
VARIANT (1)..(1): C, S, or T
VARIANT (2)..(5): Xaa can be any naturally occurring amino acid
Xaa Xaa Xaa Xaa Xaa Cys
SEQ ID NO: 326; Length: 7; Artificial Sequence; synthetic peptide
VARIANT (1)..(1): C, S, or T
VARIANT (2)..(6): Xaa can be any naturally occurring amino acid
Xaa Xaa Xaa Xaa Xaa Xaa Cys
SEQ ID NO: 327; Length: 8; Artificial Sequence; synthetic peptide
VARIANT (1)..(1): C, S, or T
VARIANT (2)..(7): Xaa can be any naturally occurring amino acid
Xaa Xaa Xaa Xaa Xaa Xaa Xaa Cys
SEQ ID NO: 328; Length: 4; Artificial Sequence; synthetic peptide
VARIANT (1)..(1): Xaa can be any naturally occurring amino acid
VARIANT (2)..(2): C, S, or T
VARIANT (3)..(3): Xaa can be any naturally occurring amino acid
Xaa Xaa Xaa Cys
SEQ ID NO: 329; Length: 5; Artificial Sequence; synthetic peptide
VARIANT (1)..(1): Xaa can be any naturally occurring amino acid
VARIANT (2)..(2): C, S, or T
VARIANT (3)..(4): Xaa can be any naturally occurring amino acid
Xaa Xaa Xaa Xaa Cys
SEQ ID NO: 330; Length: 6; Artificial Sequence; synthetic peptide
VARIANT (1)..(1): Xaa can be any naturally occurring amino acid
VARIANT (2)..(2): C, S, or T
VARIANT (3)..(5): Xaa can be any naturally occurring amino acid
Xaa Xaa Xaa Xaa Xaa Cys
SEQ ID NO: 331; Length: 7; Artificial Sequence; synthetic peptide
VARIANT (1)..(1): Xaa can be any naturally occurring amino acid
VARIANT (2)..(2): C, S, or T
VARIANT (3)..(6): Xaa can be any naturally occurring amino acid
Xaa Xaa Xaa Xaa Xaa Xaa Cys
SEQ ID NO: 332; Length: 8; Artificial Sequence; synthetic peptide
VARIANT (1)..(1): Xaa can be any naturally occurring amino acid
VARIANT (2)..(2): C, S, or T
VARIANT (3)..(7): Xaa can be any naturally occurring amino acid
Xaa Xaa Xaa Xaa Xaa Xaa Xaa Cys
SEQ ID NO: 333; Length: 9; Artificial Sequence; synthetic peptide
VARIANT (1)..(1): Xaa can be any naturally occurring amino acid
VARIANT (2)..(2): C, S, or T
VARIANT (3)..(8): Xaa can be any naturally occurring amino acid
Xaa Xaa Xaa Xaa Xaa Xaa Xaa Xaa Cys
SEQ ID NO: 334; Length: 4; Artificial Sequence; synthetic peptide
VARIANT (1)..(2): Xaa can be any naturally occurring amino acid
VARIANT (3)..(3): C, S, or T
Xaa Xaa Xaa Cys
SEQ ID NO: 335; Length: 5; Artificial Sequence; synthetic peptide
VARIANT (1)..(2): Xaa can be any naturally occurring amino acid
VARIANT (3)..(3): C, S, or T
VARIANT (4)..(4): Xaa can be any naturally occurring amino acid
Xaa Xaa Xaa Xaa Cys
SEQ ID NO: 336; Length: 6; Artificial Sequence; synthetic peptide
VARIANT (1)..(2): Xaa can be any naturally occurring amino acid
VARIANT (3)..(3): C, S, or T
VARIANT (4)..(5): Xaa can be any naturally occurring amino acid
Xaa Xaa Xaa Xaa Xaa Cys
SEQ ID NO: 337; Length: 7; Artificial Sequence; synthetic peptide
VARIANT (1)..(2): Xaa can be any naturally occurring amino acid
VARIANT (3)..(3): C, S, or T
VARIANT (4)..(6): Xaa can be any naturally occurring amino acid
Xaa Xaa Xaa Xaa Xaa Xaa Cys
SEQ ID NO: 338; Length: 8; Artificial Sequence; synthetic peptide
VARIANT (1)..(2): Xaa can be any naturally occurring amino acid
VARIANT (3)..(3): C, S, or T
VARIANT (4)..(7): Xaa can be any naturally occurring amino acid
Xaa Xaa Xaa Xaa Xaa Xaa Xaa Cys
SEQ ID NO: 339; Length: 9; Artificial Sequence; synthetic peptide
VARIANT (1)..(2): Xaa can be any naturally occurring amino acid
VARIANT (3)..(3): C, S, or T
VARIANT (4)..(8): Xaa can be any naturally occurring amino acid
Xaa Xaa Xaa Xaa Xaa Xaa Xaa Xaa Cys
SEQ ID NO: 340; Length: 10; Artificial Sequence; synthetic peptide
VARIANT (1)..(2): Xaa can be any naturally occurring amino acid
VARIANT (3)..(3): C, S, or T
VARIANT (4)..(9): Xaa can be any naturally occurring amino acid
Xaa Xaa Xaa Xaa Xaa Xaa Xaa Xaa Xaa Cys
SEQ ID NO: 341; Length: 5; Artificial Sequence; synthetic peptide
VARIANT (1)..(3): Xaa can be any naturally occurring amino acid
VARIANT (4)..(4): C, S, or T
Xaa Xaa Xaa Xaa Cys
SEQ ID NO: 342; Length: 6; Artificial Sequence; synthetic peptide
VARIANT (1)..(3): Xaa can be any naturally occurring amino acid
VARIANT (4)..(4): C, S, or T
VARIANT (5)..(5): Xaa can be any naturally occurring amino acid
Xaa Xaa Xaa Xaa Xaa Cys
SEQ ID NO: 343; Length: 7; Artificial Sequence; synthetic peptide
VARIANT (1)..(3): Xaa can be any naturally occurring amino acid
VARIANT (4)..(4): C, S, or T
VARIANT (5)..(6): Xaa can be any naturally occurring amino acid
Xaa Xaa Xaa Xaa Xaa Xaa Cys
SEQ ID NO: 344; Length: 8; Artificial Sequence; synthetic peptide
VARIANT (1)..(3): Xaa can be any naturally occurring amino acid
VARIANT (4)..(4): C, S, or T
VARIANT (5)..(7): Xaa can be any naturally occurring amino acid
Xaa Xaa Xaa Xaa Xaa Xaa Xaa Cys
SEQ ID NO: 345; Length: 9; Artificial Sequence; synthetic peptide
VARIANT (1)..(3): Xaa can be any naturally occurring amino acid
VARIANT (4)..(4): C, S, or T
VARIANT (5)..(8): Xaa can be any naturally occurring amino acid
Xaa Xaa Xaa Xaa Xaa Xaa Xaa Xaa Cys
SEQ ID NO: 346; Length: 10; Artificial Sequence; synthetic peptide
VARIANT (1)..(3): Xaa can be any naturally occurring amino acid
VARIANT (4)..(4): C, S, or T
VARIANT (5)..(9): Xaa can be any naturally occurring amino acid
Xaa Xaa Xaa Xaa Xaa Xaa Xaa Xaa Xaa Cys
SEQ ID NO: 347; Length: 11; Artificial Sequence; synthetic peptide
VARIANT (1)..(3): Xaa can be any naturally occurring amino acid
VARIANT (4)..(4): C, S, or T
VARIANT (5)..(10): Xaa can be any naturally occurring amino acid
Xaa Xaa Xaa Xaa Xaa Xaa Xaa Xaa Xaa Xaa Cys
SEQ ID NO: 348; Length: 4; Artificial Sequence; synthetic peptide
VARIANT (2)..(3): Xaa can be any naturally occurring amino acid
VARIANT (4)..(4): C, S, or T
Cys Xaa Xaa Xaa
SEQ ID NO: 349; Length: 5; Artificial Sequence; synthetic peptide
VARIANT (2)..(4): Xaa can be any naturally occurring amino acid
VARIANT (5)..(5): C, S, or T
Cys Xaa Xaa Xaa Xaa
SEQ ID NO: 350; Length: 6; Artificial Sequence; synthetic peptide
VARIANT (2)..(5): Xaa can be any naturally occurring amino acid
VARIANT (6)..(6): C, S, or T
Cys Xaa Xaa Xaa Xaa Xaa
SEQ ID NO: 351; Length: 7; Artificial Sequence; synthetic peptide
VARIANT (2)..(6): Xaa can be any naturally occurring amino acid
VARIANT (7)..(7): C, S, or T
Cys Xaa Xaa Xaa Xaa Xaa Xaa
SEQ ID NO: 352; Length: 8; Artificial Sequence; synthetic peptide
VARIANT (2)..(7): Xaa can be any naturally occurring amino acid
VARIANT (8)..(8): C, S, or T
Cys Xaa Xaa Xaa Xaa Xaa Xaa Xaa
SEQ ID NO: 353; Length: 4; Artificial Sequence; synthetic peptide
VARIANT (1)..(1): Xaa can be any naturally occurring amino acid
VARIANT (3)..(3): Xaa can be any naturally occurring amino acid
VARIANT (4)..(4): C, S, or T
Xaa Cys Xaa Xaa
SEQ ID NO: 354; Length: 5; Artificial Sequence; synthetic peptide
VARIANT (1)..(1): Xaa can be any naturally occurring amino acid
VARIANT (3)..(4): Xaa can be any naturally occurring amino acid
VARIANT (5)..(5): C, S, or T
Xaa Cys Xaa Xaa Xaa
SEQ ID NO: 355; Length: 6; Artificial Sequence; synthetic peptide
VARIANT (1)..(1): Xaa can be any naturally occurring amino acid
VARIANT (3)..(5): Xaa can be any naturally occurring amino acid
VARIANT (6)..(6): C, S, or T
Xaa Cys Xaa Xaa Xaa Xaa
SEQ ID NO: 356; Length: 7; Artificial Sequence; synthetic peptide
VARIANT (1)..(1): Xaa can be any naturally occurring amino acid
VARIANT (3)..(6): Xaa can be any naturally occurring amino acid
VARIANT (7)..(7): C, S, or T
Xaa Cys Xaa Xaa Xaa Xaa Xaa
SEQ ID NO: 357; Length: 8; Artificial Sequence; synthetic peptide
VARIANT (1)..(1): Xaa can be any naturally occurring amino acid
VARIANT (3)..(7): Xaa can be any naturally occurring amino acid
VARIANT (8)..(8): C, S, or T
Xaa Cys Xaa Xaa Xaa Xaa Xaa Xaa
SEQ ID NO: 358; Length: 9; Artificial Sequence; synthetic peptide
VARIANT (1)..(1): Xaa can be any naturally occurring amino acid
VARIANT (3)..(8): Xaa can be any naturally occurring amino acid
VARIANT (9)..(9): C, S, or T
Xaa Cys Xaa Xaa Xaa Xaa Xaa Xaa Xaa
SEQ ID NO: 359; Length: 4; Artificial Sequence; synthetic peptide
VARIANT (1)..(2): Xaa can be any naturally occurring amino acid
VARIANT (4)..(4): C, S, or T
Xaa Xaa Cys Xaa
SEQ ID NO: 360; Length: 5; Artificial Sequence; synthetic peptide
VARIANT (1)..(2): Xaa can be any naturally occurring amino acid
VARIANT (4)..(4): Xaa can be any naturally occurring amino acid
VARIANT (5)..(5): C, S, or T
Xaa Xaa Cys Xaa Xaa
SEQ ID NO: 361; Length: 6; Artificial Sequence; synthetic peptide
VARIANT (1)..(2): Xaa can be any naturally occurring amino acid
VARIANT (4)..(5): Xaa can be any naturally occurring amino acid
VARIANT (6)..(6): C, S, or T
Xaa Xaa Cys Xaa Xaa Xaa
SEQ ID NO: 362; Length: 7; Artificial Sequence; synthetic peptide
VARIANT (1)..(2): Xaa can be any naturally occurring amino acid
VARIANT (4)..(6): Xaa can be any naturally occurring amino acid
VARIANT (7)..(7): C, S, or T
Xaa Xaa Cys Xaa Xaa Xaa Xaa
SEQ ID NO: 363; Length: 8; Artificial Sequence; synthetic peptide
VARIANT (1)..(2): Xaa can be any naturally occurring amino acid
VARIANT (4)..(7): Xaa can be any naturally occurring amino acid
VARIANT (8)..(8): C, S, or T
Xaa Xaa Cys Xaa Xaa Xaa Xaa Xaa
SEQ ID NO: 364; Length: 9; Artificial Sequence; synthetic peptide
VARIANT (1)..(2): Xaa can be any naturally occurring amino acid
VARIANT (4)..(8): Xaa can be any naturally occurring amino acid
VARIANT (9)..(9): C, S, or T
Xaa Xaa Cys Xaa Xaa Xaa Xaa Xaa Xaa
SEQ ID NO: 365; Length: 10; Artificial Sequence; synthetic peptide
VARIANT (1)..(2): Xaa can be any naturally occurring amino acid
VARIANT (4)..(9): Xaa can be any naturally occurring amino acid
VARIANT (10)..(10): C, S, or T
Xaa Xaa Cys Xaa Xaa Xaa Xaa Xaa Xaa Xaa
SEQ ID NO: 366; Length: 5; Artificial Sequence; synthetic peptide
VARIANT (1)..(3): Xaa can be any naturally occurring amino acid
VARIANT (5)..(5): C, S, or T
Xaa Xaa Xaa Cys Xaa
SEQ ID NO: 367; Length: 6; Artificial Sequence; synthetic peptide
VARIANT (1)..(3): Xaa can be any naturally occurring amino acid
VARIANT (5)..(5): Xaa can be any naturally occurring amino acid
VARIANT (6)..(6): C, S, or T
Xaa Xaa Xaa Cys Xaa Xaa
SEQ ID NO: 368; Length: 7; Artificial Sequence; synthetic peptide
VARIANT (1)..(3): Xaa can be any naturally occurring amino acid
VARIANT (5)..(6): Xaa can be any naturally occurring amino acid
VARIANT (7)..(7): C, S, or T
Xaa Xaa Xaa Cys Xaa Xaa Xaa
SEQ ID NO: 369; Length: 8; Artificial Sequence; synthetic peptide
VARIANT (1)..(3): Xaa can be any naturally occurring amino acid
VARIANT (5)..(7): Xaa can be any naturally occurring amino acid
VARIANT (8)..(8): C, S, or T
Xaa Xaa Xaa Cys Xaa Xaa Xaa Xaa
SEQ ID NO: 370; Length: 9; Artificial Sequence; synthetic peptide
VARIANT (1)..(3): Xaa can be any naturally occurring amino acid
VARIANT (5)..(8): Xaa can be any naturally occurring amino acid
VARIANT (9)..(9): C, S, or T
Xaa Xaa Xaa Cys Xaa Xaa Xaa Xaa Xaa
SEQ ID NO: 371; Length: 10; Artificial Sequence; synthetic peptide
VARIANT (1)..(3): Xaa can be any naturally occurring amino acid
VARIANT (5)..(9): Xaa can be any naturally occurring amino acid
VARIANT (10)..(10): C, S, or T
Xaa Xaa Xaa Cys Xaa Xaa Xaa Xaa Xaa Xaa
SEQ ID NO: 372; Length: 11; Artificial Sequence; synthetic peptide
VARIANT (1)..(3): Xaa can be any naturally occurring amino acid
VARIANT (5)..(10): Xaa can be any naturally occurring amino acid
VARIANT (11)..(11): C, S, or T
Xaa Xaa Xaa Cys Xaa Xaa Xaa Xaa Xaa Xaa Xaa
SEQ ID NO: 373; Length: 5; Artificial Sequence; synthetic peptide
VARIANT (1)..(1): X = basic amino acid
VARIANT (3)..(4): Xaa can be any naturally occurring amino acid
Xaa Cys Xaa Xaa Cys
SEQ ID NO: 374; Length: 6; Artificial Sequence; synthetic peptide
VARIANT (1)..(1): X = basic amino acid
VARIANT (2)..(2): X = basic amino acid
VARIANT (4)..(5): Xaa can be any naturally occurring amino acid
Xaa Xaa Cys Xaa Xaa Cys
SEQ ID NO: 375; Length: 6; Artificial Sequence; synthetic peptide
VARIANT (1)..(1): X = basic amino acid
VARIANT (3)..(5): Xaa can be any naturally occurring amino acid
Xaa Cys Xaa Xaa Xaa Cys
SEQ ID NO: 376; Length: 7; Artificial Sequence; synthetic peptide
VARIANT (1)..(1): X = basic amino acid
VARIANT (2)..(2): X = basic amino acid
VARIANT (4)..(6): Xaa can be any naturally occurring amino acid
Xaa Xaa Cys Xaa Xaa Xaa Cys
SEQ ID NO: 377; Length: 4; Artificial Sequence; synthetic peptide
VARIANT (1)..(1): X = C, S, or T and modified to carry an acetyl,
methyl, ethyl or propionyl group
VARIANT (2)..(3): Xaa can be any naturally occurring amino acid
Xaa Xaa Xaa Cys
SEQ ID NO: 378; Length: 5; Artificial Sequence; synthetic peptide
VARIANT (1)..(1): X = C, S, or T and modified to carry an acetyl,
methyl, ethyl or propionyl group
VARIANT (2)..(4): Xaa can be any naturally occurring amino acid
Xaa Xaa Xaa Xaa Cys
SEQ ID NO: 379; Length: 6; Artificial Sequence; synthetic peptide
VARIANT (1)..(1): X = C, S, or T and modified to carry an acetyl,
methyl, ethyl or propionyl group
VARIANT (2)..(5): Xaa can be any naturally occurring amino acid
Xaa Xaa Xaa Xaa Xaa Cys
SEQ ID NO: 380; Length: 7; Artificial Sequence; synthetic peptide
VARIANT (1)..(1): X = C, S, or T and modified to carry an acetyl,
methyl, ethyl or propionyl group
VARIANT (2)..(6): Xaa can be any naturally occurring amino acid
Xaa Xaa Xaa Xaa Xaa Xaa Cys
SEQ ID NO: 381; Length: 8; Artificial Sequence; synthetic peptide
VARIANT (1)..(1): X = C, S, or T and modified to carry an acetyl,
methyl, ethyl or propionyl group
VARIANT (2)..(7): Xaa can be any naturally occurring amino acid
Xaa Xaa Xaa Xaa Xaa Xaa Xaa Cys
SEQ ID NO: 382; Length: 4; Artificial Sequence; synthetic peptide
VARIANT (1)..(1): X = C, S, or T
VARIANT (2)..(3): Xaa can be any naturally occurring amino acid
VARIANT (4)..(4): X = C modified to carry an acetyl, methyl, ethyl or
propionyl group
Xaa Xaa Xaa Xaa
SEQ ID NO: 383; Length: 5; Artificial Sequence; synthetic peptide
VARIANT (1)..(1): X = C, S, or T
VARIANT (2)..(4): Xaa can be any naturally occurring amino acid
VARIANT (5)..(5): X = C modified to carry an acetyl, methyl, ethyl or
propionyl group
Xaa Xaa Xaa Xaa Xaa
SEQ ID NO: 384; Length: 6; Artificial Sequence; synthetic peptide
VARIANT (1)..(1): X = C, S, or T
VARIANT (2)..(5): Xaa can be any naturally occurring amino acid
VARIANT (6)..(6): X = C modified to carry an acetyl, methyl, ethyl or
propionyl group
Xaa Xaa Xaa Xaa Xaa Xaa
SEQ ID NO: 385; Length: 7; Artificial Sequence; synthetic peptide
VARIANT (1)..(1): X = C, S, or T
VARIANT (2)..(6): Xaa can be any naturally occurring amino acid
VARIANT (7)..(7): X = C modified to carry an acetyl, methyl, ethyl or
propionyl group
Xaa Xaa Xaa Xaa Xaa Xaa Xaa
SEQ ID NO: 386; Length: 8; Artificial Sequence; synthetic peptide
VARIANT (1)..(1): X = C, S, or T
VARIANT (2)..(7): Xaa can be any naturally occurring amino acid
VARIANT (8)..(8): X = C modified to carry an acetyl, methyl, ethyl or
propionyl group
Xaa Xaa Xaa Xaa Xaa Xaa Xaa Xaa
SEQ ID NO: 387; Length: 4; Artificial Sequence; synthetic peptide
VARIANT (1)..(1): X = C modified to carry an acetyl, methyl, ethyl or
propionyl group
VARIANT (2)..(3): Xaa can be any naturally occurring amino acid
VARIANT (4)..(4): X = C, S, or T
Xaa Xaa Xaa Xaa
SEQ ID NO: 388; Length: 5; Artificial Sequence; synthetic peptide
VARIANT (1)..(1): X = C modified to carry an acetyl, methyl, ethyl or
propionyl group
VARIANT (2)..(4): Xaa can be any naturally occurring amino acid
VARIANT (5)..(5): X = C, S, or T
Xaa Xaa Xaa Xaa Xaa
SEQ ID NO: 389; Length: 6; Artificial Sequence; synthetic peptide
VARIANT (1)..(1): X = C modified to carry an acetyl, methyl, ethyl or
propionyl group
VARIANT (2)..(5): Xaa can be any naturally occurring amino acid
VARIANT (6)..(6): X = C, S, or T
Xaa Xaa Xaa Xaa Xaa Xaa
SEQ ID NO: 390; Length: 7; Artificial Sequence; synthetic peptide
VARIANT (1)..(1): X = C modified to carry an acetyl, methyl, ethyl or
propionyl group
VARIANT (2)..(6): Xaa can be any naturally occurring amino acid
VARIANT (7)..(7): X = C, S, or T
Xaa Xaa Xaa Xaa Xaa Xaa Xaa
SEQ ID NO: 391; Length: 8; Artificial Sequence; synthetic peptide
VARIANT (1)..(1): X = C modified to carry an acetyl, methyl, ethyl or
propionyl group
VARIANT (2)..(7): Xaa can be any naturally occurring amino acid
VARIANT (8)..(8): X = C, S, or T
Xaa Xaa Xaa Xaa Xaa Xaa Xaa Xaa
SEQ ID NO: 392; Length: 4; Artificial Sequence; synthetic peptide
VARIANT (2)..(3): Xaa can be any naturally occurring amino acid
VARIANT (4)..(4): X = C, S, or T and modified to carry an acetyl,
methyl, ethyl or propionyl group
Cys Xaa Xaa Xaa
SEQ ID NO: 393; Length: 5; Artificial Sequence; synthetic peptide
VARIANT (2)..(4): Xaa can be any naturally occurring amino acid
VARIANT (5)..(5): X = C, S, or T and modified to carry an acetyl,
methyl, ethyl or propionyl group
Cys Xaa Xaa Xaa Xaa
SEQ ID NO: 394; Length: 6; Artificial Sequence; synthetic peptide
VARIANT (2)..(5): Xaa can be any naturally occurring amino acid
VARIANT (6)..(6): X = C, S, or T and modified to carry an acetyl,
methyl, ethyl or propionyl group
Cys Xaa Xaa Xaa Xaa Xaa
SEQ ID NO: 395; Length: 7; Artificial Sequence; synthetic peptide
VARIANT (2)..(6): Xaa can be any naturally occurring amino acid
VARIANT (7)..(7): X = C, S, or T and modified to carry an acetyl,
methyl, ethyl or propionyl group
Cys Xaa Xaa Xaa Xaa Xaa Xaa
SEQ ID NO: 396; Length: 8; Artificial Sequence; synthetic peptide
VARIANT (2)..(7): Xaa can be any naturally occurring amino acid
VARIANT (8)..(8): X = C, S, or T and modified to carry an acetyl,
methyl, ethyl or propionyl group
Cys Xaa Xaa Xaa Xaa Xaa Xaa Xaa
SEQ ID NO: 397; Length: 20; Artificial Sequence; synthetic peptide
Ala Gly Gly Leu Tyr Glu Tyr Val Phe Ser Pro Asp Val Glu Phe Lys Arg
Arg Phe Lys
SEQ ID NO: 398; Length: 20; Artificial Sequence; synthetic peptide
Ala Gly Gly Leu Tyr Glu Tyr Val Phe Cys Pro Asp Val Glu Phe Lys Arg
Arg Phe Lys
SEQ ID NO: 399; Length: 23; Artificial Sequence; synthetic peptide
Lys Val Ala Met Val Cys Thr Arg Lys Ile Ser Ile Ala Lys Ser Val Phe
Tyr Ile Ala Ala Lys Lys
SEQ ID NO: 400; Length: 18; Artificial Sequence; synthetic peptide
Ser Ile Met Val Ala Phe Lys Gly Val Trp Thr Gln Ala Phe Trp Lys Ala
Val
SEQ ID NO: 401; Length: 23; Artificial Sequence; synthetic peptide
His Cys Pro Tyr Cys Ser Ile Met Val Ala Phe Lys Gly Val Trp Thr Gln
Ala Phe Trp Lys Ala Val

1. An isolated immunogenic peptide with a length of between 12 and 50 amino acids, said immunogenic peptide comprising:
an oxidoreductase motif with the sequence Zₘ-[CST]-Xₙ-C- or Zₘ-C-Xₙ-[CST]-, wherein n is an integer chosen from: 2, 0, 1 or 3, wherein m is an integer selected from 1, 0, 2, or 3, wherein X is any amino acid, wherein Z is any amino acid, in which C stands for cysteine, S for serine, T for threonine; and
a T-cell epitope from the Aquaporin 4 (AQP4) antigen;
wherein said oxidoreductase motif and said epitope are separated by a linker sequence of between 0 to 7 amino acids, preferably wherein said epitope comprises a sequence selected from the group consisting of SEQ ID NO: 1 to 135.

2. The peptide according to claim 1, wherein said epitope is not a mouse Aquaporin-4 epitope, more specifically, said epitope is not mouse AQP4 epitope SIMVAFKGVWTQAFWKAV and said immunogenic peptide is not HCPYCSIMVAFKGVWTQAFWKAV.

3. The peptide according to claim 1,wherein said linker-epitope-flanker is selected from the group consisting of: TRKISIAKSVFYIAA, TRKISIAKSVFYIAAKK and TRKISIAKSVFYIAAKKK or wherein said linker-epitope is selected from the group consisting of: EYVFSPDVEFKRRFK and EYVFCPDVEFKRRFK

4. (canceled)

5. The peptide according to claim 1,wherein said oxidoreductase motif is selected from the following amino acid motifs:
Zₘ-[CST]-Xₙ-C- or Zₘ-C-Xₙ-[CST]-, (a)
wherein n is 0, and wherein m is an integer selected from 0, 1, or 2,
wherein Z is any amino acid, preferably a basic amino acid preferably selected from: H, K, R, and a non-natural basic amino acid such as L-ornithine, more preferably K or H, most preferably K;
Zₘ-[CST]-Xₙ-C- or Zₘ-C-Xₙ-[CST]-, (b)
wherein n is 1, wherein X is any amino acid, preferably a basic amino acid selected from: H, K, R, and a non-natural basic amino acid such as L-ornithine, more preferably K or R,
wherein m is an integer selected from 0, 1, or 2,
wherein Z is any amino acid, preferably a basic amino acid preferably selected from: H, K, R, and a non-natural basic amino acid such as L-ornithine, more preferably K or H, most preferably K;
Zₘ-[CST]-Xₙ-C- or Zₘ-C-Xₙ-[CST]-, (c)
wherein n is 2, thereby creating an internal X¹X² amino acid couple within the oxidoreductase motif, wherein X is any amino acid, preferably wherein at least one X is a basic amino acid selected from: H, K, R, and a non-natural basic amino acid such as L-ornithine, more preferably K or R,
wherein m is an integer selected from 0, 1, or 2,
wherein Z is any amino acid, preferably a basic amino acid preferably selected from: H, K, R, and a non-natural basic amino acid, such as L-ornithine, more preferably K or H, most preferably H;
Zₘ-[CST]-Xₙ-C- or Zₘ-C-Xₙ-[CST]- (d)
wherein n is 3, thereby creating an internal X¹X²X³ amino acid stretch within the oxidoreductase motif, wherein X is any amino acid, preferably wherein at least one X is a basic amino acid selected from: H, K, R, and a non-natural basic amino acid such as L-ornithine, more preferably K or R,
wherein m is an integer selected from 0, 1, or 2,
wherein Z is any amino acid, preferably a basic amino acid preferably selected from: H, K, R, and a non-natural basic amino acid as defined herein, such as L-ornithine, more preferably K or H; or
Zₘ-[CST]-Xₙ-C- or Zₘ-C-Xₙ-[CST]-, (h)
wherein n is 0 to 3 and wherein m is 0, and wherein one of the C or [CST] residues has been modified so as to carry an acetyl, methyl, ethyl or propionyl group, either on the N-terminal amide of the amino acid residue of the motif or on the C-terminal carboxy group.

6. The peptide according to claim 1, wherein said T-cell epitope is flanked at its C-terminus by a sequence of between 0 and 7 amino acids, and/or said immunogenic peptide additionally comprises one or more K or D amino acid residuc(s) flanking the epitope at the C-terminus.

7. (canceled)

8. The peptide according to claim 1, wherein the oxidoreductase motif
(a) has a sequence of Zₘ-C-XX-C-, with Z being a basic amino acid;
(b) has a sequence of Zₘ-C-XX-C-, with Z being a basic amino acid selected from the group consisting of K and H, m being 0, 1, or 2;
(c) has a sequence selected from the group consisting of: CPYC (SEQ ID NO: 157), HCPYC (SEQ ID NO: 158), KHCPYC (SEQ ID NO: 159), KCPYC (SEQ ID NO: 160), RCPYC (SEQ ID NO: 161), KKCPYC (SEQ ID NO: 162), KRCPYC (SEQ ID NO: 163), CHGC (SEQ ID NO: 164), HCGHC (SEQ ID NO: 165), KCGHC (SEQ ID NO: 166), KHCGHC (SEQ ID NO: 167), RCGHC (SEQ ID NO: 168), KKCGHC (SEQ ID NO: 169), and KRCGHC (SEQ ID NO: 170);
(d) has a sequence selected from the group consisting of: HCPYC (SEQ ID NO: 158) or KHCPYC (SEQ ID NO: 159);
(c) has a sequence of Zₘ-C-X-C-, with Z being a basic amino acid, and m being 0, 1, or 2;
(f) has a sequence of Zₘ-C-X-C-, with Z being a basic amino acid selected from the group consisting of K and H, and m being 0, 1, or 2;
(g) has a sequence of Zₘ-C-X-C-, with Z being a basic amino acid selected from the group consisting of K and H, m being 0, 1, or 2, and X being R;
(c) has a sequence selected from the group consisting of: KCC, CRC, KCRC (SEQ ID NO:

243), HCRC (SEQ ID NO: 320) and KHCRC (SEQ ID NO: 321);
(f) has the sequence KCRC (SEQ ID NO: 243);
(g) has a sequence selected from the group consisting of: CRPYC (SEQ ID NO: 250), KCRPYC (SEQ ID NO: 251), KHCRPYC (SEQ ID NO: 252), RCRPYC (SEQ ID NO: 253), HCRPYC (SEQ ID NO: 254), CPRYC (SEQ ID NO: 255), KCPRYC (SEQ ID NO: 256), RCPRYC (SEQ ID NO: 257), HCPRYC (SEQ ID NO: 258), CPYRC (SEQ ID NO: 259), KCPYRC (SEQ ID NO: 260), RCPYRC (SEQ ID NO: 261), HCPYRC (SEQ ID NO: 262), CKPYC (SEQ ID NO: 263), KCKPYC (SEQ ID NO: 264), RCKPYC (SEQ ID NO: 265), HCKPYC (SEQ ID NO: 266), CPKYC (SEQ ID NO: 267), KCPKYC (SEQ ID NO: 268), RCPKYC (SEQ ID NO: 269), HCPKYC (SEQ ID NO: 270), CPYKC (SEQ ID NO: 271), KCPYKC (SEQ ID NO: 272), RCPYKC (SEQ ID NO: 273), and HCPYKC (SEQ ID NOs: 274); and/or
(h) has the sequence KCRPYC (SEQ ID NO: 251).

9-10. (canceled)

11. The peptide according to claim 1, wherein said immunogenic peptide
(a) comprises or consists of any one of the amino sequences selected from the group consisting of SEQ ID NO: 137 to 156;
(b) has the following sequence: HCPYCTRKISIAKSVFYIAAKKK (SEQ ID NO: 146) or HCPYCEYVFSPDVEFKRRFK (SEQ ID NO: 154).

12-16. (canceled)

17. A polynucleotide encoding the peptide according to claim 1, wherein said polynucleotide is selected from the group comprising DNA, pDNA, cDNA, RNA, and mRNA or modified versions thereof, preferably a non-immunogenic mRNA comprising N(1)-methyl-pseudouridine (ml).

18. A pharmaceutical composition comprising the peptide according to claim 1, or a polynucleotide encoding the peptide according to claim 1.

19-21. (canceled)

22. An in vitro method for the generation of a population of cytolytic CD4+ T cells or NKT cells, against APC presenting AQP4 epitopes, comprising the steps of:
providing peripheral blood cells;
contacting said cells in vitro with the peptide of claim 1, or a polynucleotide encoding the peptide of claim 1; and
expanding said cells in the presence of IL-2.

23. A population of cytolytic CD4+ T cells or NKT cells, against APC presenting AQP4 epitopes, obtained by the method of claim 22.

24. (canceled)

25. A method of treating of, ameliorating the symptoms of, and/or preventing of a disease or disorder by administering to a subject in need thereof an effective dose of the population of cytolytic CD4+ T cells or NKT cells according to claim 23, wherein the disease or disorder
(a) is an anti-AQP4 disease or a Neuromyelitis Optica Spectrum Disorder;
(b) is NMO; Optic Neuritis; Devic's disease; AQP4-positive Optic-Spinal MS (OSMS); Longitudinally Extensive (Transverse) Myelitis; or AQP4-positive myelitis;
(c) is associated with lesions in specific brain areas such as the hypothalamus, periventricular nucleus, and brainstem; and/or
(d) is Tumefactive demyelination or lesions.

26. The method according to claim 25, wherein said treatment is combined with, preferably preceded by an antibody depletion therapy.

27-29. (canceled)

30. A method for treating of, ameliorating the symptoms of, and/or preventing a Neuromyelitis Optica Spectrum Disorder in a subject, comprising the step of administering a therapeutically effective amount of the peptide according to claim 1 or a polynucleotide encoding the peptide according to claim 1 to a subject in need thereof.

31. The method according to claim 30, wherein said disorder is selected from the group consisting of: NMO; Optic Neuritis; Devic's disease; AQP4-positive Optic-Spinal MS (OSMS); Longitudinally Extensive (Transverse) Myelitis; AQP4-positive myelitis, preferably associated with lesions in specific brain areas such as the hypothalamus, periventricular nucleus, and brainstem; and Tumefactive demyelination or lesions, preferably a disorder caused or aggravated by AQP4 auto-antigens and/or anti-AQP4 antibodies, most preferably Neuromyelitis Optica (NMO).

32. (canceled)

33. An in vitro method for detecting MHC class II restricted CD4+ T cells specific for a AQP4 antigen in a sample comprising the steps of;
contacting a subject sample with a complex of an isolated MHC class II molecules and a peptide according to claim 1;
detecting CD4+ T cells by measuring the binding of said complex with cells in said sample, wherein the binding of the complex to a cell is indicative for the presence of CD4+ T cells specific for a AQP4 antigen in said sample.

34. A method for treating of, ameliorating the symptoms of, and/or preventing a Neuromyelitis Optica Spectrum Disorder in a subject in need thereof, comprising the step of administering to the subject a therapeutically effective amount of the peptide according to claim 1 or a polynucleotide encoding the peptide according to claim 1, and an antibody having B cell depleting activity, wherein said antibody and said immunogenie peptide or polynucleotide er cells are administered either simultaneously, sequentially or separately.

35. The method according to claim 34, wherein said antibody having B cell depleting activity is administrated before said peptide or polynucleotide.

36. The method according to claim 34 or 35, wherein said antibody having B cell depleting activity
(a) is selected from one that binds an antigen selected from the group consisting of CD10, CD19, CD20, CD21, CD22, CD23, CD24, CD37, CD53, CD72, CD73, CD74, CDw75, CDw76, CD77, CDw78, CD79a, CD79b, CD80 (B7.1), CD81, CD82, CD83, CDw84, CD85 and CD86 (B7.2);
(b) is selected from one that binds CD19,
(c) is Inebilizumab (MEDI-551);
(d) is selected from one that binds CD20, or
(e) is Rituximab or Ublituximab (LFB-R603, TGT-1101, TGTX-1101).

37-38. (canceled)

39. A pharmaceutical preparation, combination, composition, or kit-of-parts, comprising the peptide according to claim 1, or a polynucleotide encoding the peptide according to claim 1, and an antibody having B cell depleting activity.

40-46. (canceled)
